(19) United States
(12) Patent Application Publication
(10) Pub. No.: US 2025/0320211 A1
(43) Pub. Date: Oct. 16, 2025

(54) COMPOUND, COMPOSITION, AND ELECTROPHOTOGRAPHIC PHOTORECEPTOR

(71) Applicant: MITSUBISHI CHEMICAL CORPORATION, Tokyo (JP)

(72) Inventors: Tsukasa HASEGAWA, Tokyo (JP); Manuel Emilio OTERO RAMIREZ, Tokyo (JP); Akira ANDO, Tokyo (JP); Hideki GOROUMARU, Tokyo (JP)

(73) Assignee: MITSUBISHI CHEMICAL CORPORATION, Tokyo (JP)

(21) Appl. No.: 19/251,782

(22) Filed: Jun. 26, 2025

Related U.S. Application Data
(63) Continuation of application No. PCT/JP2023/046986, filed on Dec. 27, 2023.

(30) Foreign Application Priority Data
Dec. 28, 2022 (JP) 2022-212000
Dec. 28, 2022 (JP) 2022-212003

(51) Int. Cl. C07D 471/06; G03G 5/147

(52) U.S. Cl. CPC C07D 471/06; G03G 5/14726

(57) ABSTRACT

The present disclosure relates to a compound having two or more polymerizable functional groups in one molecule and represented by the following formula (1).

### CROSS-REFERENCE TO RELATED APPLICATIONS

The present application is a continuation of International Patent Application PCT/JP2023/046986, filed Dec. 27, 2023, which is based on and claims the benefit of priority to Japanese Patent Application No. 2022-212000, and Japanese Patent Application No. 2022-212003 filed on Dec. 28, 2022. The entire contents of these applications are incorporated herein by reference.

### TECHNICAL FIELD

The present invention relates to a compound having an electron transporting structure and a composition containing the compound. The compound and composition of the present invention are useful as a material for forming a protective layer of an electrophotographic photoreceptor, for example, a copier, a printer, or the like.

The present invention also relates to an electrophotographic photoreceptor using the compound.

### BACKGROUND ART

In a printer, a copier and the like, when light is applied to a charged organic photoconductor (OPC) drum, an electrostatic latent image is formed because the charge is eliminated from the part, and an image can be obtained because the toner adheres to the electrostatic latent image. Thus, in a device using electrophotographic technique, the photoreceptor is the key member.

Because there is considerable room for material selection for such a kind of organic photoconductor and because the characteristics of the photoreceptor are easily regulated, a “function-separated photoreceptor” in which the functions of generating and transporting the charge are assigned to different compounds has become the mainstream. For example, a single layer electrophotographic photoreceptor (called a “single layer type photoreceptor” hereinafter having a charge generating material (CGM) and a charge transporting material (CTM) in the same layer and a laminate type electrophotographic photoreceptor (called a “laminate type photoreceptor” hereinafter) obtained by laminating a charge generation layer containing a charge generating material (CGM) and a charge transport layer containing a charge transporting material (CTM) are known. Moreover, the charging methods of a photoreceptor include a negatively charging method for negatively charging a surface of a photoreceptor and a positively charging method for positively charging a surface of a photoreceptor.

Combinations of the layer configuration of a photoreceptor and the charging method which are currently used are “a negatively charged laminate type photoreceptor” and “a positively charged single layer photoreceptor”.

A “negatively charged laminate type photoreceptor” has a configuration obtained by providing an undercoat layer (UCL) composed of a resin or the like on a conductive base such as an aluminum tube or the like, providing a charge generation layer (CGL) composed of a charge generating material (CGM), a resin and the like thereon, and further providing a charge transport layer (CTL) composed of a hole transporting material (HTM), a resin and the like thereon.

On the other hand, a “positively charged single layer photoreceptor” has a configuration obtained by providing an undercoat layer (UCL) composed of a resin or the like on a conductive base such as an aluminum tube or the like, and providing a single layer photosensitive layer composed of a charge generating material (CGM), a hole transporting material (HTM), an electron transporting material (ETM), a resin and the like thereon (for example, see Patent Literature 1).

In both photoreceptors, by charging the surface of the photoreceptor by a corona discharging method or a contact method and then exposing the photoreceptor to neutralize the charge on the surface, an electrostatic latent image is formed due to the potential difference from the surrounding surface. Then, a toner is brought into contact with the photoreceptor surface to form a toner image corresponding to the electrostatic latent image, and print is finished by transferring/heat-melt fixing the image on paper or the like.

As described above, a photosensitive layer is formed on a conductive support in the basic configuration of an electrophotographic photoreceptor, but a protective layer is sometimes provided on the photosensitive layer for the purpose of improving the abrasion resistance or the like.

As a technique for improving the mechanical strength or the abrasion resistance of the surface of a photoreceptor, a photoreceptor obtained by forming a layer containing a compound having a chain-polymerizable functional group as a binder resin on the outermost layer of the photoreceptor and applying energy such as heat, light, radiation, and the like to the layer to polymerize the compound to form a cured resin layer is disclosed (for example, see Patent Literatures 1 and 2).

Such a protective layer is generally formed by dissolving a curable composition containing a compound having a chain-polymerizable functional group in an organic solvent to prepare a coating liquid for forming a protective layer, and then coating the coating liquid on the surface of the photoreceptor to form a protective layer.

### CITATION LIST

### Patent Literature

Patent Literature 1: U.S. Pat. No. 9,417,538B
Patent Literature 2: WO2010/035683

### SUMMARY OF INVENTION

### Technical Problem

As described above, it is known to provide a protective layer to improve the abrasion resistance of the photoreceptor. In particular, a protective layer using a curable compound (a compound having a chain-polymerizable functional group) has particularly excellent mechanical strength and can provide a good protective effect.

On the other hand, from the viewpoint of improving the electrical properties of the photoreceptor, the protective layer is required to have good electron transporting property as well as mechanical strength. In order to improve the electron transporting property of the photoreceptor, it is effective to add a compound having an electron transporting structure to the protective layer.

It has been found, however, that some compounds having an electron transporting structure have insufficient solubility in organic solvents used to prepare a coating liquid for forming a protective layer, and further insufficient electrical properties of the photoreceptor obtained by forming the protective layer.

An object of the present invention is to provide a compound having an electron transporting structure, which has excellent electron transporting property and solubility in organic solvents, and is possible to manufacture a photoreceptor having excellent electrical properties, particularly residual potential property and potential retention rate.

### Solution to Problem

The present inventors have found that a specific compound having a perylene diimide skeleton into which a halogen atom has been introduced as an electron transporting structure has excellent electron transporting property, and can be used as a protective layer to produce a photoreceptor having excellent electrical properties, particularly residual potential property and potential retention rate, and also has excellent solubility in organic solvents.

The present invention has been achieved based on these findings, and has the following gist.
[1] A compound having two or more polymerizable functional groups in one molecule and represented by the following formula (1),
   wherein the polymerizable functional groups are selected from the following formulae (M1) to (M7):

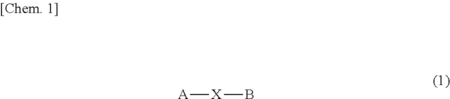

(In the formula (1), X represents a perylene diimide skeleton represented by the following formula (2).
   A and B represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents, an aromatic heterocyclic group which may have one or more substituents, or a group represented by the following formula (3). A and B may be the same or different from each other.)

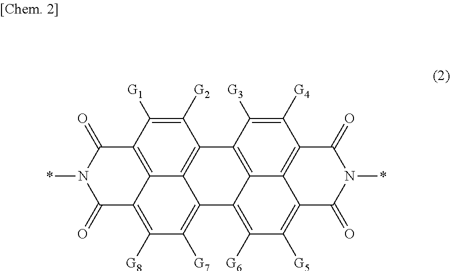

(In the formula (2), G₁ to G₈ each independently represent a hydrogen atom, a deuterium atom, a halogen atom, an alkyl group which may have one or more substituents, or an alkoxy group which may have one or more substituents. However, at least one of G₁ to G₈ is a halogen atom. * represents a bond to A or B.)

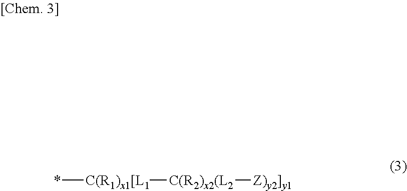

(In the formula (3), * represents a bond to X.
   R₁ and R₂ each independently represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents, or an aromatic heterocyclic group which may have one or more substituents.
   L₁ and L₂ each independently represent a direct bond or a divalent group.
   Z represents a hydrogen atom, an alkyl group, an alkoxy group, an amide group, or a polymerizable functional group.
   x1+y1=3, where x1 is an integer from 0 to 2 and y1 is an integer from 1 to 3; x2+y2=3, where x2 is an integer from 0 to 2 and y2 is an integer from 1 to 3; wherein when x1 is an integer of 2 or more, R₁ may be the same or different from each other; when y1 is an integer of 2 or more, each of R₂, x2, y2, L₁, L₂ and Z may be the same or different from each other; when x2 is an integer of 2 or more, R₂ may be the same or different from each other; and, when y2 is an integer of 2 or more, each of L₂ and Z may be the same or different from each other.)

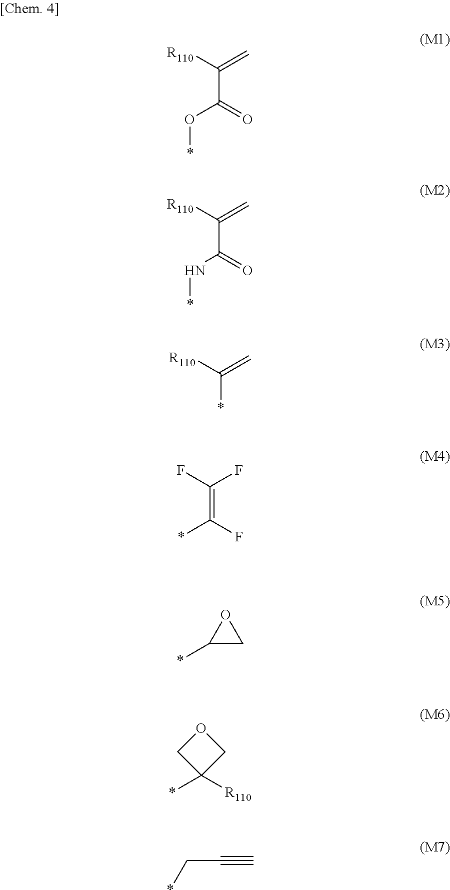

(In the formulae (M1) to (M7), R₁₁₀ represents a hydrogen atom or an alkyl group which may have one or more substituents, and * represents a bonding position.)
[2] The compound according to [1], wherein L₁ and L₂ in the formula (3) are each independently an alkylene group, a divalent group having a ketone group, a divalent group having an ether bond, a divalent group having an ester bond, or a group wherein these are linked.
[3] A compound having at least one structure represented by the following formula (3A) in one molecule, and represented by the following formula (1).

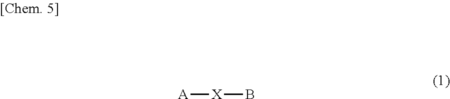

(In the formula (1), X represents a perylene diimide skeleton represented by the following formula (2).
   A and B represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents, an aromatic heterocyclic group which may have one or more substituents, a group represented by the following formula (3), or a group represented by the following formula (3B). A and B may be the same or different from each other.)

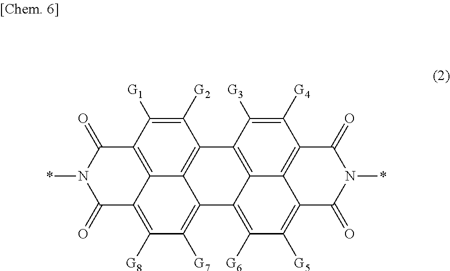

(In the formula (2), G₁ to G₈ each independently represent a hydrogen atom, a deuterium atom, a halogen atom, an alkyl group which may have one or more substituents, an alkoxy group which may have one or more substituents, or a group represented by the following formula (3B). However, at least one of G₁ to G₈ is a halogen atom. * represents a bond to A or B.)

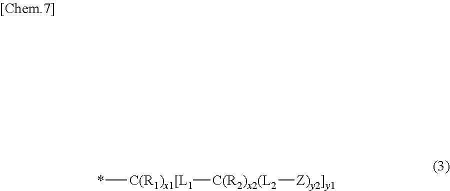

(In the formula (3), * represents a bond to X.
   R₁ and R₂ each independently represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents, an aromatic heterocyclic group which may have one or more substituents, or a group represented by the following formula (3B).
   L₁ and L₂ each independently represent a direct bond or a divalent group.
   Z represents a hydrogen atom, an alkyl group, an alkoxy group, an amide group, or a polymerizable functional group.
   x1+y1=3, where x1 is an integer from 0 to 2 and y1 is an integer from 1 to 3; x2+y2=3, where x2 is an integer from 0 to 2 and y2 is an integer from 1 to 3; wherein when x1 is an integer of 2 or more, R₁ may be the same or different from each other; when y1 is an integer of 2 or more, each of R₂, x2, y2, L₁, L₂ and Z may be the same or different from each other; when x2 is an integer of 2 or more, R₂ may be the same or different from each other; and when y2 is an integer of 2 or more, each of L₂ and Z may be the same or different from each other.)

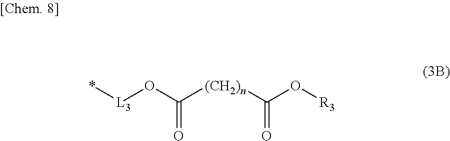

(In the formula (3B), * represents a bond to any atom in the formulae (1) to (3), and n is an integer of 1 or more.
   L₃ represents a direct bond or a divalent group.
   R₃ represents a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents, an aromatic heterocyclic group which may have one or more substituents, or a polymerizable functional group.

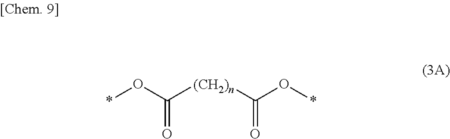

(In the formula (3A), * represents a bond to any atom in the formulae (1) to (3), and n is an integer of 1 or more.)
[4] The compound according to [3], wherein at least one of L₁ and L₂ in the formula (3) is a divalent group, and the divalent group is a group represented by the formula (3A).
[5] The compound according to [3] or [4], wherein the compound has at least one polymerizable functional group in one molecule.
[6] The compound according to [5], wherein the polymerizable functional group is selected from the following formulae (M1) to (M7).

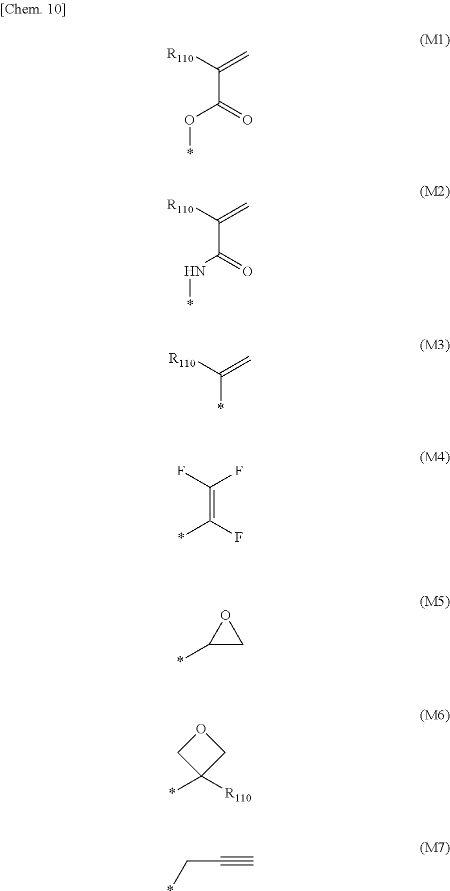

(In the formulae (M1) to (M7), R₁₁₀ represents a hydrogen atom or an alkyl group which may have one or more substituents, and * represents a bonding position.)
[7] The compound according to any one of [1] to [6], wherein at least one of A and B in the formula (1) is a group represented by the above formula (3).
[8] The compound according to any one of [1] to [7], wherein two or more of G₁ to G₈ are halogen atoms.
[9] The compound according to [8], wherein four or more of G₁ to G₈ are halogen atoms.
[10] The compound according to any one of [1] to [9], wherein R₁ in the formula (3) is an alkyl group which may have one or more substituents.
[11] A composition comprising the compound according to any one of [1] to [10] and a polymerizable compound having no electron transporting skeleton.
[12] The composition according to [11], further comprising an electron donating compound.
[13] An electrophotographic photoreceptor comprising:
   a conductive support;
   a photosensitive layer and a protective layer formed in this order on the conductive support,
   wherein the protective layer comprises a polymer of the compound according to any one of [1] to [10].
[14] An electrophotographic photoreceptor comprising:
   a conductive support;
   a photosensitive layer and a protective layer formed in this order on the conductive support,
   wherein the protective layer comprises the compound according to any one of [3] to [10].

### Advantageous Effects of Invention

The compound of the present invention has excellent electron transporting property and excellent solubility in organic solvents, particularly alcohol-based solvents that are commonly used in preparing coating liquids for forming protective layers.

By using the compound of the present invention as a curable compound for forming a protective layer of an electrophotographic photoreceptor, it is possible to manufacture a photoreceptor having excellent electrical properties such as residual potential property and potential retention rate with good solvent solubility and good workability.

### BRIEF DESCRIPTION OF DRAWINGS

FIG. 1 is a FIGURE schematically illustrating an example configuration of an image formation device which can be configured using the electrophotographic photoreceptor according to an example of the present invention.

### DESCRIPTION OF EMBODIMENTS

Embodiments for carrying out the present invention (embodiments of the invention below) will be explained in detail below. Here, the present invention is not limited to the embodiments below and can be carried out with various modifications in the scope of the gist of the invention.

### <<Compound>>

The compound according to the first embodiment of the present invention is a compound having two or more polymerizable functional groups in one molecule and represented by the following formula (1), wherein the polymerizable functional groups have a halogenated perylene diimide skeleton, which is an electron transporting skeleton, selected from the following formulae (M1) to (M7).

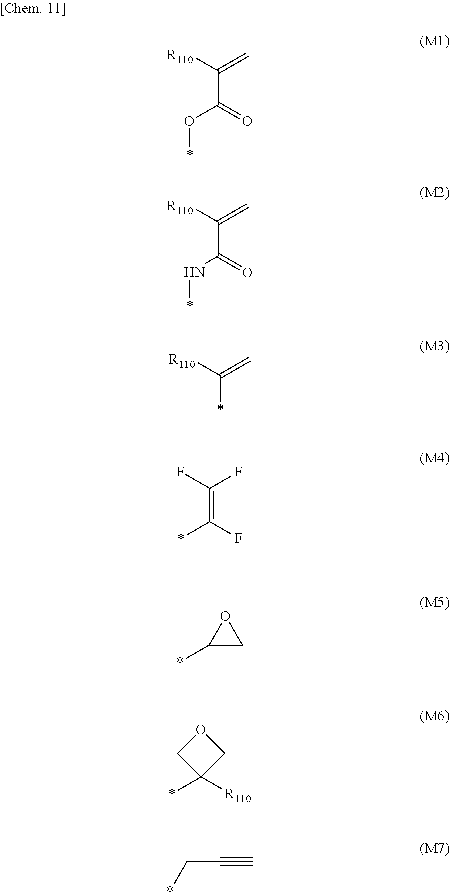

(In the formulae (M1) to (M7), R₁₁₀ represents a hydrogen atom or an alkyl group which may have one or more substituents, and * represents a bonding position.)

The compound according to the second embodiment of the present invention is a compound having at least one structure represented by the following formula (3A) (hereinafter, also referred to as “linking group (3A)”) in one molecule, and having a halogenated perylene diimide skeleton, which is an electron transporting skeleton, represented by the following formula (1).

In the present invention, the term “electron transporting compound” means a compound having electron transporting properties, in other words, a compound having an electron transporting skeleton.

Hereinafter, the compound according to the first embodiment of the present invention and the compound according to the second embodiment of the present invention will be collectively referred to as the “compound of the present invention”.

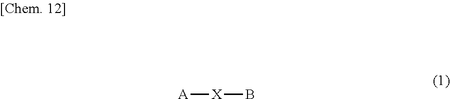

(In the formula (1), X represents a perylene diimide skeleton represented by the following formula (2).

A and B represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents, an aromatic heterocyclic group which may have one or more substituents, or a group represented by the following formula (3). In the compound according to the second embodiment of the present invention, A and B may further represent a group represented by the following formula (3B). A and B may be the same or different from each other.)

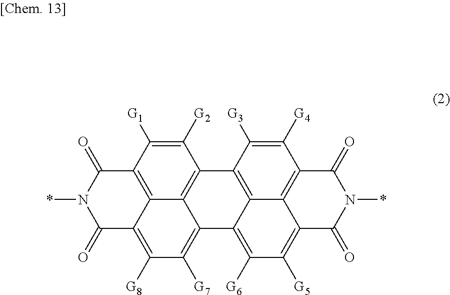

(In the formula (2), G₁ to G₈ each independently represent a hydrogen atom, a deuterium atom, a halogen atom, an alkyl group which may have one or more substituents, or an alkoxy group which may have one or more substituents. However, at least one of G₁ to G₈ is a halogen atom. * represents a bond to A or B.)

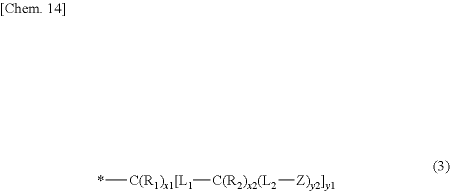

(In the formula (3), * represents a bond to X.

R₁ and R₂ each independently represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents, or an aromatic heterocyclic group which may have one or more substituents.

L₁ and L₂ each independently represent a direct bond or a divalent group.

Z represents a hydrogen atom, an alkyl group, an alkoxy group, an amide group, or a polymerizable functional group.

x1+y1=3, where x1 is an integer from 0 to 2 and y1 is an integer from 1 to 3; x2+y2=3, where x2 is an integer from 0 to 2 and y2 is an integer from 1 to 3; wherein when x1 is an integer of 2 or more, R₁ may be the same or different from each other; when y1 is an integer of 2 or more, each of R₂, x2, y2, L₁, L₂ and Z may be the same or different from each other; when x2 is an integer of 2 or more, R₂ may be the same or different from each other; and, when y2 is an integer of 2 or more, each of L₂ and Z may be the same or different from each other.)

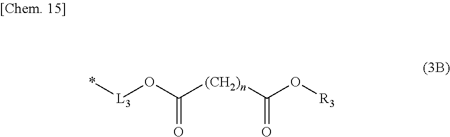

(In the formula (3B), * represents a bond to any atom in the formulae (1) to (3), and n is an integer of 1 or more.

L₃ represents a direct bond or a divalent group.

R₃ represents a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents, an aromatic heterocyclic group which may have one or more substituents, or a polymerizable functional group.

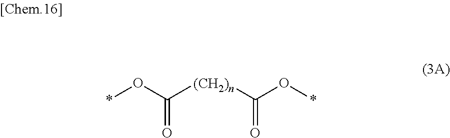

(In the formula (3A), * represents a bond to any atom in the formulae (1) to (3), and n is an integer of 1 or more.)

In the present invention, the term “which may have one or more substituents” means that the group can have one or more substituents and has the meanings including both having one or more substituents and having no substituent.

In the compounds of the present invention, examples of the substituents of the alkyl groups which may have one or more substituents and the like in the above formulae (2) and (3) include an alkyl group, an alkoxy group, an aryloxy group, a heteroaryloxy group, an alkoxycarbonyl group, a dialkylamino group, a diarylamino group, an arylalkylamino group, an acyl group, a haloalkyl group, an alkylthio group, an arylthio group, a silyl group, a siloxy group, an acryloyl group, a methacryloyl group, an acrylamide group, an aromatic hydrocarbon group, an aromatic heterocyclic groups, and the like. From the viewpoint of solubility, when these groups have a substituent, the substituent is preferably an alkyl group, and more preferably has no substituent.

### <Mechanism>

The mechanism by which the compounds of the present invention exhibit the effect of having excellent solubility in organic solvents is considered to be as follows.

The perylene diimide skeleton has high electron affinity and excellent electron transporting property, but due to its large n-conjugated skeleton, it has poor solubility in organic solvents, especially in alcohol-based solvents.

However, by introducing at least one halogen atom into this perylene diimide skeleton, the perylene diimide skeleton is twisted, improving its solubility in organic solvents such as alcohol-based solvents, making it possible to apply it to a coating liquid for forming a protective layer.

In the compound according to the first embodiment of the present invention, by making it have a structure having two or more polymerizable functional groups in one molecule, it becomes possible to suppress the aggregation of the perylene diimide skeleton, and therefore the solubility in organic solvents such as alcohol-based solvents is further improved.

In the compound according to the second embodiment of the present invention, by introducing a side chain having a specific structure so as to be bonded to the nitrogen atom of this perylene diimide skeleton, it becomes possible to improve the affinity with alcohol-based solvents and suppress the aggregation of the perylene diimide skeleton, and therefore the solubility in organic solvents such as alcohol-based solvents is further improved, making it easy to apply it to a coating liquid for forming a protective layer.

In any of the compounds according to the embodiments, the electron affinity can be increased and the electron transporting property can be improved by introducing a halogen atom, particularly a chlorine atom, into the perylene diimide skeleton. Therefore, by forming a protective layer for a photoconductor using the compound of the present invention, an electrophotographic photoreceptor having excellent electrical properties such as residual potential property and potential retention rate can be provided.

In particular, by introducing a side chain having a polymerizable functional group represented by the formula (3) into the compound according to the first embodiment of the present invention, the solubility in organic solvents, particularly alcohol-based solvents, can be further improved, and a protective layer having excellent mechanical strength can be formed by polymerizing and curing during the formation of the protective layer.

Furthermore, the compound according to the second embodiment of the present invention preferably has a polymerizable functional group, and the side chain having the polymerizable functional group preferably has a branched structure. By having a polymerizable functional group, the compound of the present invention functions as a curable compound during the formation of the protective layer, and a protective layer having excellent mechanical strength can be formed. In addition, it is considered that the branching of the side chain having a polymerizable functional group results in significant steric hindrance of the compound of the present invention reduces crystallinity, resulting in further improved solubility in organic solvents, particularly alcohol-based solvents.

### <X>

X in the formula (1) represents a halogen atom-containing perylene diimide skeleton represented by the formula (2). In the halogen atom-containing perylene diimide skeleton represented by the formula (2), G₁ to G₈ are each independently a hydrogen atom, a deuterium atom, a halogen atom, an alkyl group which may have one or more substituents, or an alkoxy group which may have one or more substituents. In the compound according to the second embodiment of the present invention, G₁ to G₈ may further be a group represented by the formula (3B). However, in both the compounds according to the first and second embodiments of the present invention, at least one of G₁ to G₈ is a halogen atom.

Examples of the halogen atoms of G₁ to G₈ include one or more of a chlorine atom, a fluorine atom, a bromine atom, and an iodine atom. From the viewpoints of stability and electron transporting property of the compound, a chlorine atom is preferable.

From the viewpoint of solubility in an organic solvent, the halogen atom-containing perylene diimide skeleton represented by the formula (2) preferably has at least two halogen atoms, and preferably has at least four halogen atoms. There is no particular upper limit on the number of halogen atoms, and all of G₁ to G₈ may be halogen atoms, but usually the number of halogen atoms is preferably four or less.

In the halogen atom-containing perylene diimide skeleton represented by the formula (2), it is preferable that the halogen atoms of G₁ to G₈ are located at symmetrical positions, and it is particularly preferable that G₂, G₃, G₆, and G₇ are halogen atoms, especially chlorine atoms, from the viewpoints of stability and electron transporting property of the compound.

In the compound according to the first embodiment of the present invention, it is preferable that the ones other than the halogen atoms of G₁ to G₈ are each independently a hydrogen atom, an alkyl group which may have one or more substituents, or an alkoxy group which may have one or more substituents, and it is particularly preferable that they are hydrogen atoms.

In the compound according to the second embodiment of the present invention, it is preferable that the ones other than the halogen atoms of G₁ to G₈ are each independently a hydrogen atom, an alkyl group which may have one or more substituents, an alkoxy group which may have one or more substituents, or a group represented by the formula (3B), and it is particularly preferable that they are hydrogen atoms.

### <A and B>

A and B in the formula (1) each independently represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents, an aromatic heterocyclic group which may have one or more substituents, or a group represented by the above mentioned formula (3). In the compound according to the second embodiment of the present invention, A and B may further represent a group represented by the formula (3B).

In the compound according to the first embodiment of the present invention, from the viewpoints of solubility in organic solvents and curability, A and B are each preferably independently an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, an acyl group which may have one or more substituents, or a group represented by the formula (3), more preferably an alkoxy group which may have one or more substituents, an alkoxycarbonyl group which may have one or more substituents, an acyl group which may have one or more substituents, or a group represented by the formula (3), and it is particularly preferable that at least one of A and B is a group represented by the formula (3).

In the compound according to the second embodiment of the present invention, from the viewpoints of solubility in organic solvents and curability, A and B are each preferably independently an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, an acyl group which may have one or more substituents, a group represented by the formula (3), or a group represented by the formula (3B), more preferably an alkoxy group which may have one or more substituents, an alkoxycarbonyl group which may have one or more substituents, an acyl group which may have one or more substituents, a group represented by the formula (3), and particularly preferably a group represented by the formula (3).

A and B may be the same or different from each other, but are preferably the same from the viewpoints of solubility in organic solvents and curability.

### <Group Represented by the Formula (3)>

In the formula (3), R₁ and R₂ each independently represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents, or an aromatic heterocyclic group which may have one or more substituents. In the compound according to the second embodiment of the present invention, R₁ and R₂ may further be a group represented by the formula (3B).

From the viewpoint of solubility in organic solvents, R₁ is preferably an alkyl group which may have one or more substituents. From the viewpoint of curability, R₂ is preferably a hydrogen atom.

L₁ and L₂ each independently represent a direct bond or a divalent group.

Z represents a hydrogen atom, an alkyl group, an alkoxy group, an amide group, or a polymerizable functional group.

x1+y1=3, where x1 is an integer from 0 to 2 and y1 is an integer from 1 to 3; x2+y2=3, where x2 is an integer from 0 to 2 and y2 is an integer from 1 to 3; wherein when x1 is an integer of 2 or more, R₁ may be the same or different from each other; when y1 is an integer of 2 or more, each of R₂, x2, y2, L₁, L₂ and Z may be the same or different from each other; when x2 is an integer of 2 or more, R₂ may be the same or different from each other; and, when y2 is an integer of 2 or more, each of L₂ and Z may be the same or different from each other.

From the viewpoints of solubility in organic solvents and curability, y1 is preferably 1 or 2, and therefore x1 is preferably 2 or 1.

When x1=1, R₁ is preferably a hydrogen atom, an alkyl group which may have one or more substituents, an alkoxy group which may have one or more substituents, an alkoxycarbonyl group which may have one or more substituents, or an acyl group which may have one or more substituents, and more preferably an alkyl group which may have one or more substituents, and even more preferably a straight chain alkyl group or branched alkyl group having 4 or more carbon atoms.

When x1=2, each of the two R₁ is preferably independently a hydrogen atom, a straight chain or branched alkyl group, an alkoxy group which may have one or more substituents, an alkoxycarbonyl group which may have one or more substituents, or an acyl group which may have one or more substituents, and more preferably one R₁ is a hydrogen atom and the other R₁ is a straight chain or branched alkyl group having 4 or more carbon atoms.

In the compound according to the first embodiment of the present invention, L₁ and L₂ each independently represent a direct bond or a divalent group, however from the viewpoint of solubility in organic solvents, it is preferable that L₁ and L₂ each independently represent an alkylene group, a divalent group having a ketone group, a divalent group having an ether bond, a divalent group having an ester bond, or a group wherein these are linked, and more preferably an alkylene group, a divalent group having an ether bond, a divalent group having an ester bond, or a group wherein these are linked, and particularly preferably an alkylene group or a divalent group having an ester bond.

In the compound according to the second embodiment of the present invention, the divalent groups that L₁ and L₂ can take are preferably, from the viewpoint of film forming property, an alkylene group, a divalent group having a ketone group, a divalent group having an ether bond, a divalent group having an ester bond, or a group wherein these are linked (among these, the linking group (3A) is included in the group formed by linking an alkylene group and an ester group). From the viewpoint of solubility in organic solvents, it is particularly preferable that both L₁ and L₂ are linking groups (3A).

In the formula (3A), n is an integer of 1 or more, and is particularly preferably 2 or more, and is preferably 8 or less, and particularly preferably 4 or less. When n is equal to or more than the above lower limit, the solubility in organic solvents is excellent. When n is equal to or less than the above upper limit, the electron transporting property is excellent.

Z represents a hydrogen atom, an alkyl group, an alkoxy group, an amide group, or a polymerizable functional group.

Among these, from the viewpoints of solubility in organic solvents and curability, Z is preferably an amide group or a polymerizable functional group, and more preferably a polymerizable functional group.

The above mentioned polymerizable functional group may be an acryloyl group which may have one or more substituents, a methacryloyl group which may have one or more substituents, an acrylamide group which may have one or more substituents, or a methacrylamide group which may have one or more substituents. Among these, an acryloyl group which may have one or more substituents, or a methacryloyl group which may have one or more substituents is preferred, and the groups represented by the following formulae (P-1) to (P-5) are more preferred, and the group represented by the following formula (P-3) is even more preferred.

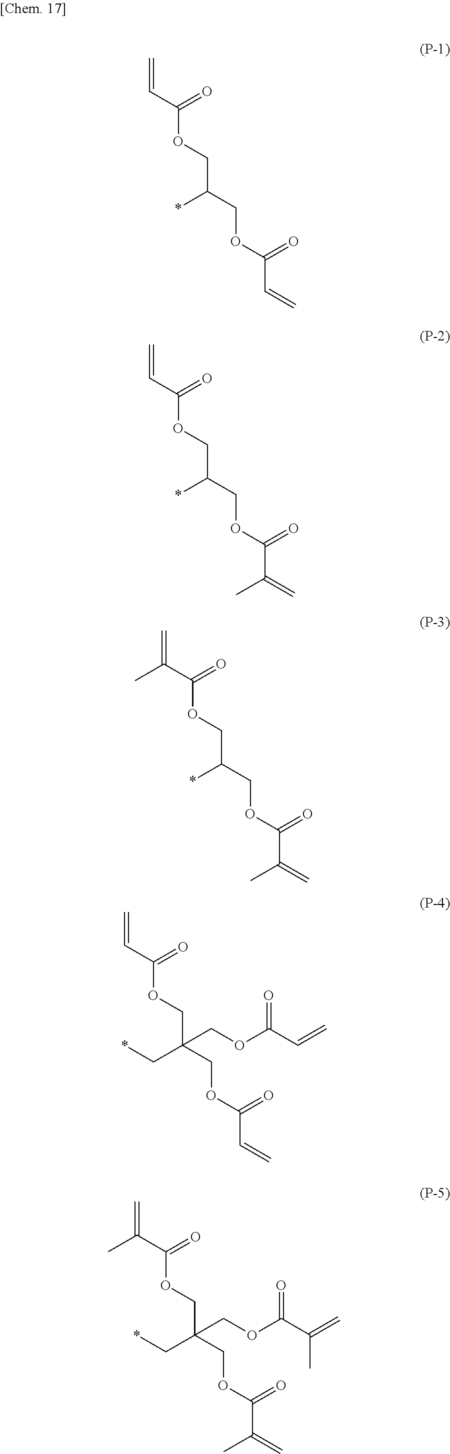

In the above formulae (P-1) to (P-5), * represents a bond to L₂.

From the viewpoint of curability, y2 is preferably 2, and therefore x2 is preferably 1.

R₂ is preferably a hydrogen atom, an alkyl group which may have one or more substituents, an alkoxy group which may have one or more substituents, an alkoxycarbonyl group which may have one or more substituents, or an acyl group which may have one or more substituents. From the viewpoint of curability, R₂ is more preferably a hydrogen atom.

When y2 is 2, the two (L₂-Z) s may be the same or different from each other, but it is preferable that they are the same from the viewpoint of solubility in organic solvents.

In the formula (3), Z is a polymerizable functional group, and y2 and/or y1 are 2, so that the side chain having the polymerizable functional group in the compound of the present invention is branched, and therefore the solubility in organic solvents is improved, which is preferable. In other words, because the side chain having the polymerizable functional group is branched, the steric hindrance of the compound of the present invention becomes significant, and the crystallinity decreases, so that the solubility in organic solvents, particularly alcohol-based solvents, is improved.

### <Group Represented by the Formula (3B)>

In the above formula (3B), n is an integer of 1 or more. In particular, it is preferably 2 or more, while it is preferably 8 or less, and particularly preferably 4 or less. When n is equal to or more than the above lower limit, the solubility in organic solvents is excellent. When n is equal to or less than the above upper limit, the electron transporting property is excellent.

L₃ represents a direct bond or a divalent group. From the viewpoint of film forming property, the divalent group is preferably an alkylene group, a divalent group having a ketone group, a divalent group having an ether bond, a divalent group having an ester bond, or a group wherein these are linked, and from the viewpoint of solubility in organic solvents, it is particularly preferable that L₃ is a divalent group having an ether bond, a divalent group having an ester bond, or a group wherein these are linked.

R₃ represents a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents, an aromatic heterocyclic group which may have one or more substituents, or a polymerizable functional group. Among these, from the viewpoint of solubility in an organic solvent, an alkyl group which may have one or more substituents, an alkoxy group which may have one or more substituents, or a polymerizable functional group is preferable.

### <Polymerizable Functional Group>

The compound according to the first embodiment of the present invention has two or more polymerizable functional groups.

When there are two or more polymerizable functional groups, it is possible to form a protective layer having excellent mechanical strength by polymerizing and curing during the formation of the protective layer, and this is preferable.

In the compound according to the first embodiment of the present invention, the number of the polymerizable functional groups may be two or more in the compound. From the viewpoints of solubility in organic solvents and curability, the number of the polymerizable functional groups is preferably three or more, and more preferably four or more. On the other hand, from the viewpoint of stability of the compound, the number of the polymerizable functional groups in the compound is preferably 12 or less, more preferably 10 or less, and even more preferably 8 or less.

The compound according to the second embodiment of the present invention preferably has at least one polymerizable functional group. By having a polymerizable functional group, it is possible to form a protective layer having excellent mechanical strength.

From the viewpoints of film forming property and curability, the number of polymerizable functional groups in the compound according to the second embodiment of the present invention is preferably two or more, more preferably three or more, and even more preferably four or more. On the other hand, from the viewpoint of stability of the compound, the number of polymerizable functional groups in the compound according to the second embodiment of the present invention is preferably 12 or less, more preferably 10 or less, and more preferably 8 or less.

The polymerizable functional group may be any functional group having polymerizability, and is not particularly limited. Examples of the polymerizable functional group include polymerizable functional groups represented by the following formulae (M1) to (M7).

In the compound according to the first embodiment of the present invention, the polymerizable functional group is selected from the following formulae (M1) to (M7). Among these, from the viewpoints of chemical stability, polymerization reactivity, and hardness of the film after film formation, the formulae (M1), (M2), and (M4) to (M7) are preferred, and the formula (M1) or the formula (M2) is more preferred.

In the compound according to the second embodiment of the present invention, from the viewpoints of chemical stability, polymerization reactivity, and hardness of the film after film formation, the formulae (M1), (M2), and (M4) to (M7) are preferred, and the formula (M1) or the formula (M2) is more preferred.

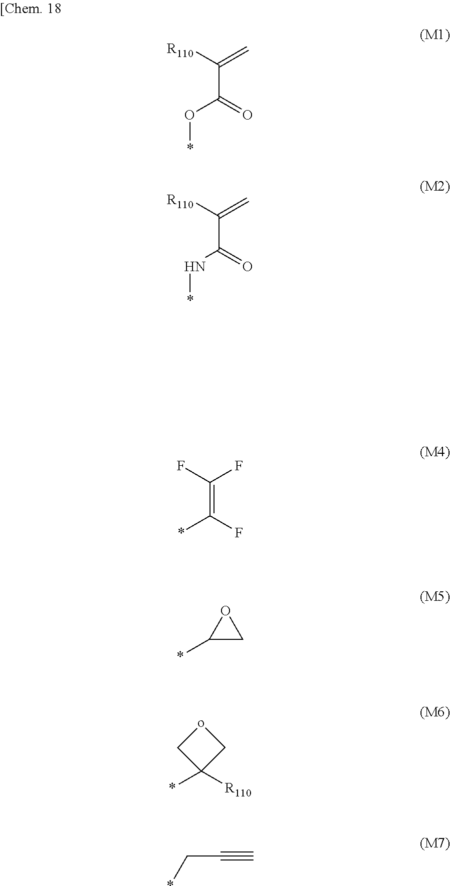

(In the formulae (M1), (M2), and (M4) to (M7), R₁₁₀ represents a hydrogen atom or an alkyl group which may have one or more substituents, and * represents a bonding position.)

R₁₁₀ in the above formulae (M1) to (M7) is preferably a hydrogen atom or an alkyl group which has no substituent, more preferably a hydrogen atom or an alkyl group having 1 to 10 carbon atoms, and particularly preferably a hydrogen atom or a methyl group.

The polymerizable functional groups possessed by the compound of the present invention do not all need to be the same and may be different. However, from the viewpoint of curability, it is preferable that they are the same.

### SPECIFIC EXAMPLES

Specific examples of the compounds according to the present invention include the following examples, but are not limited to the following compounds.

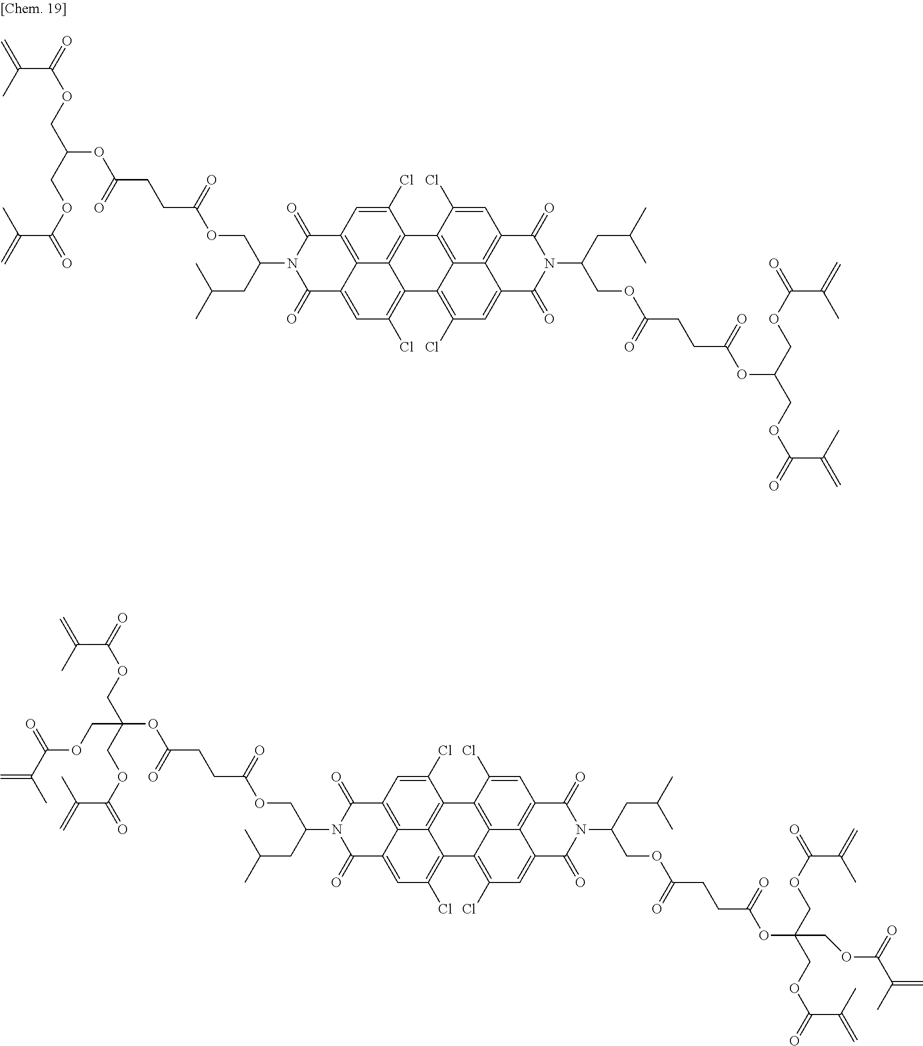

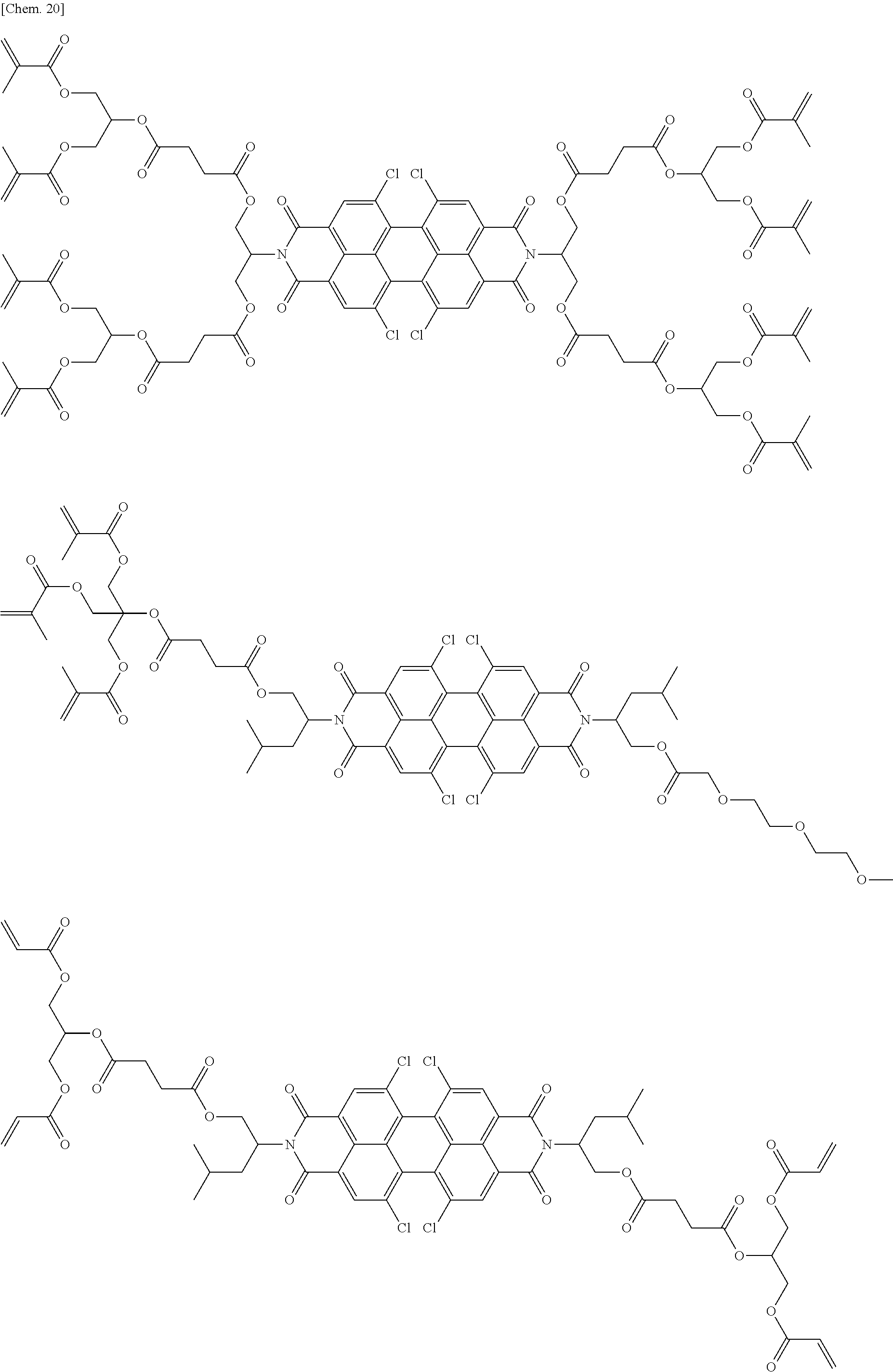

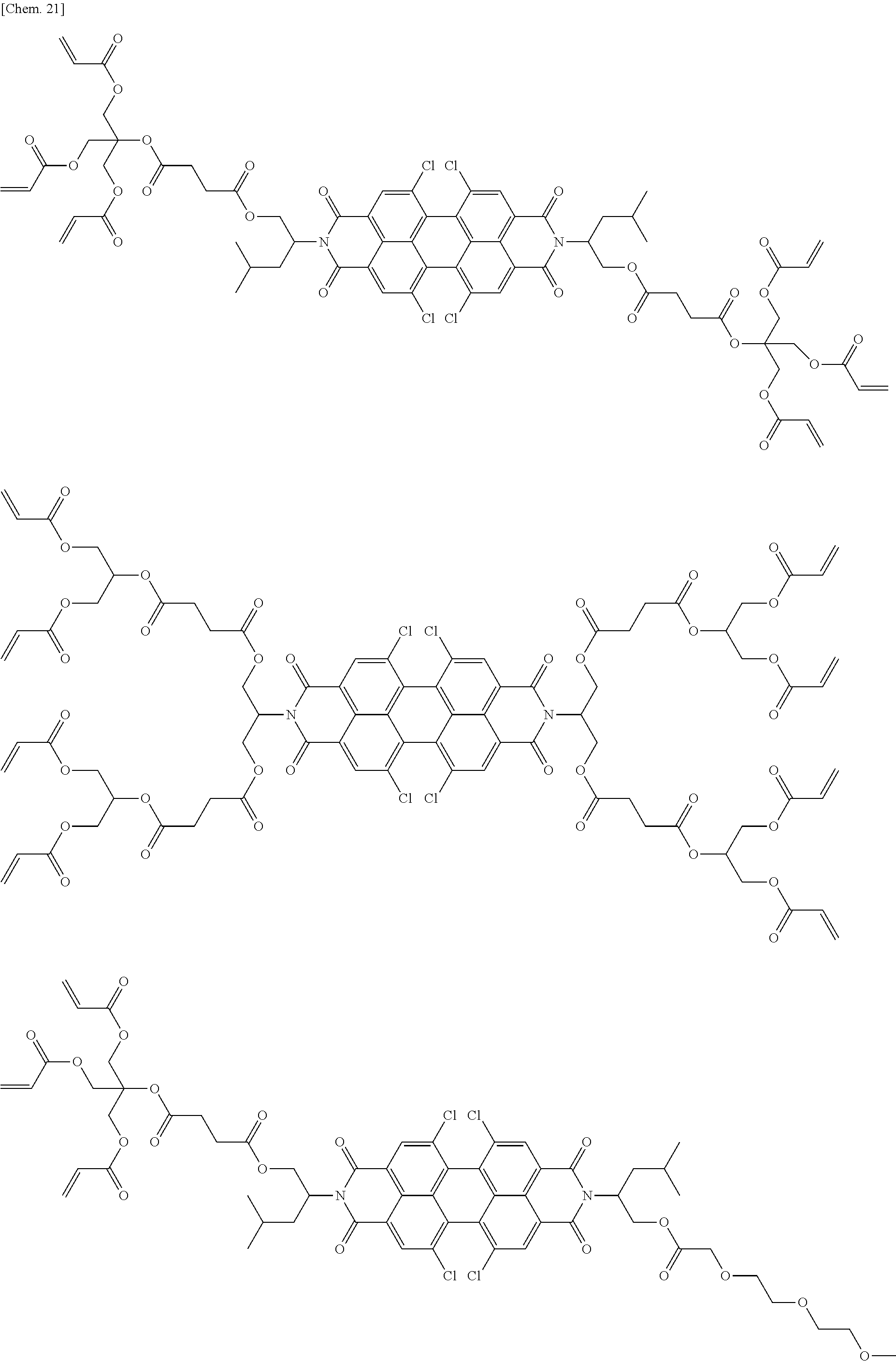

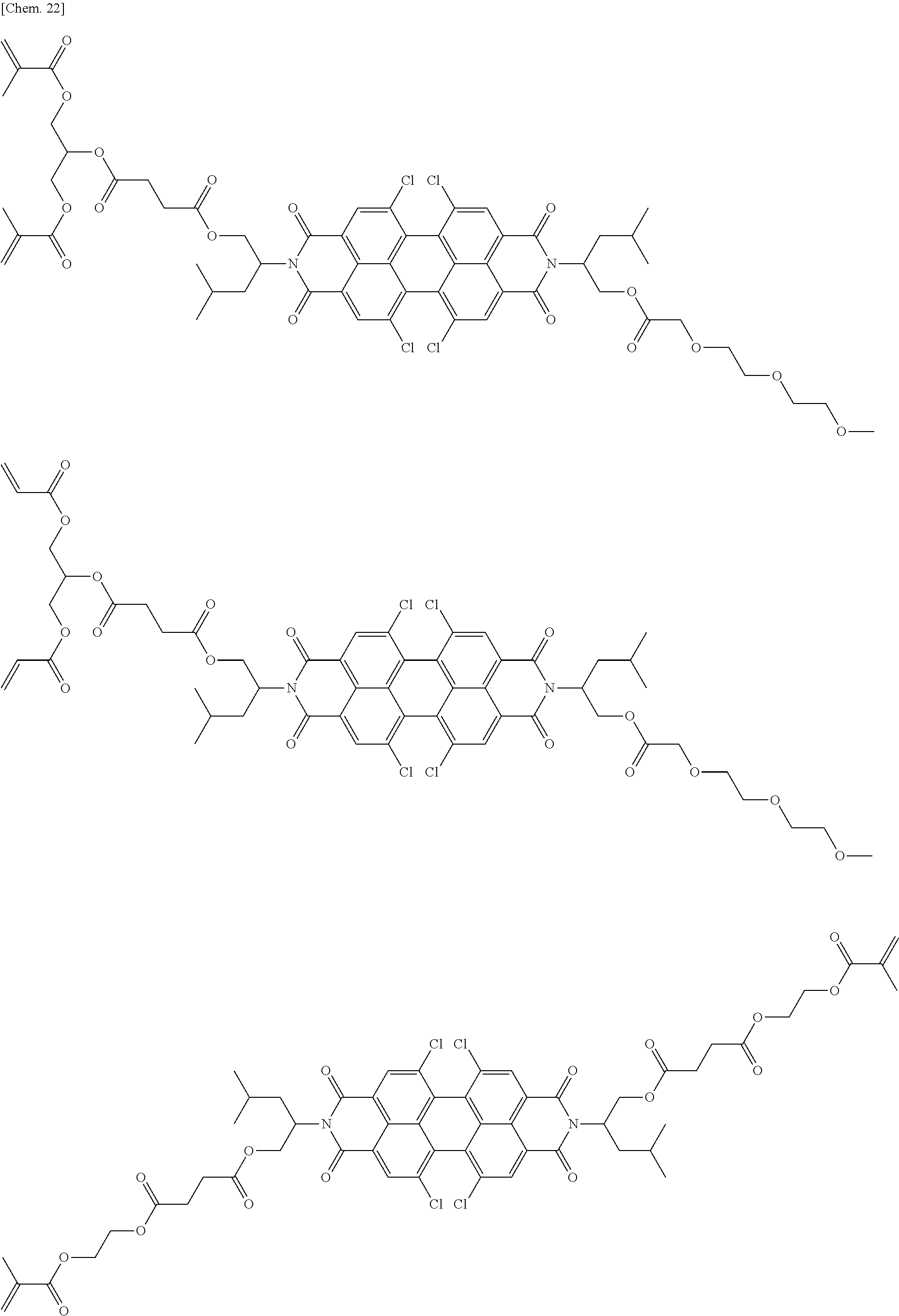

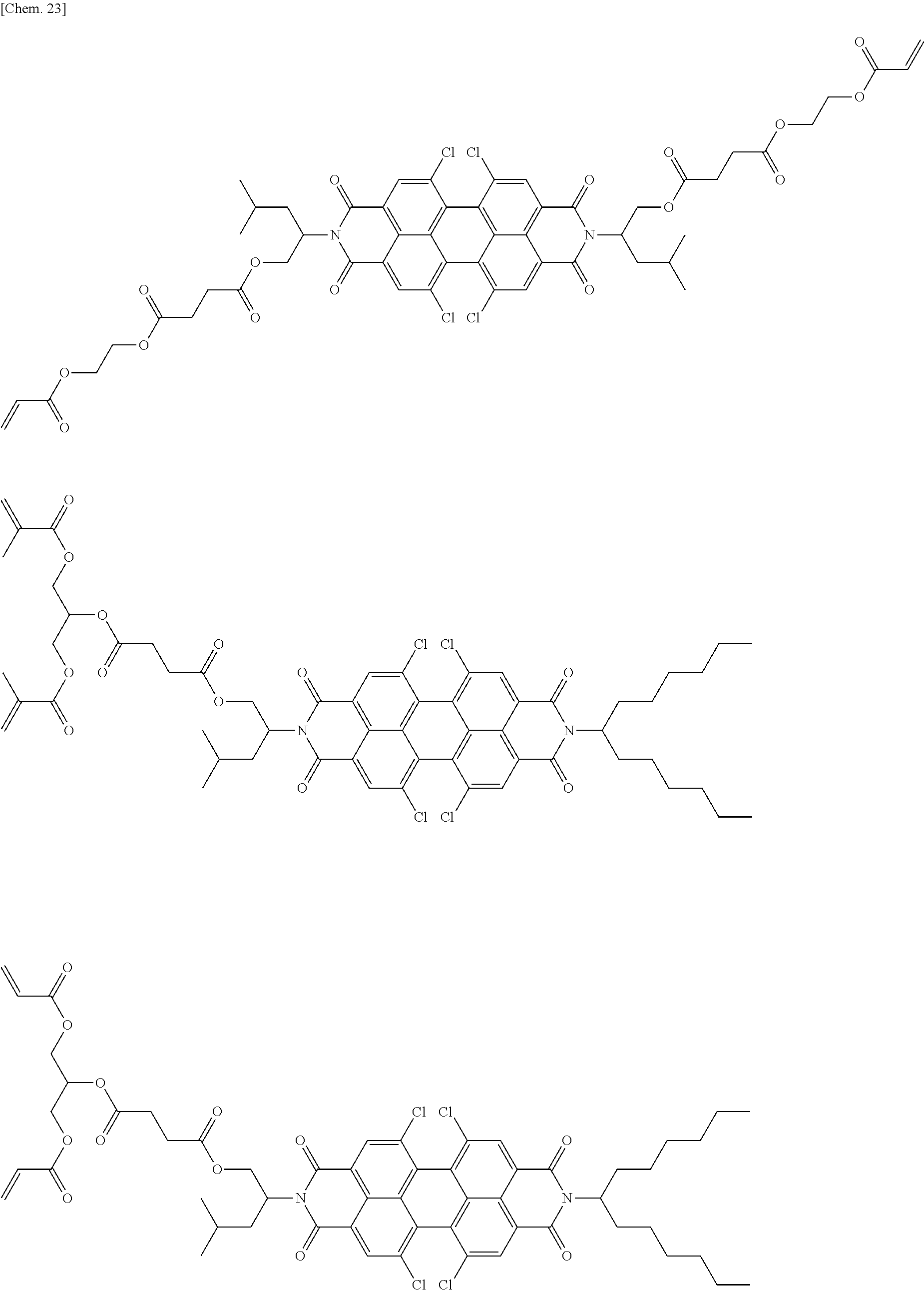

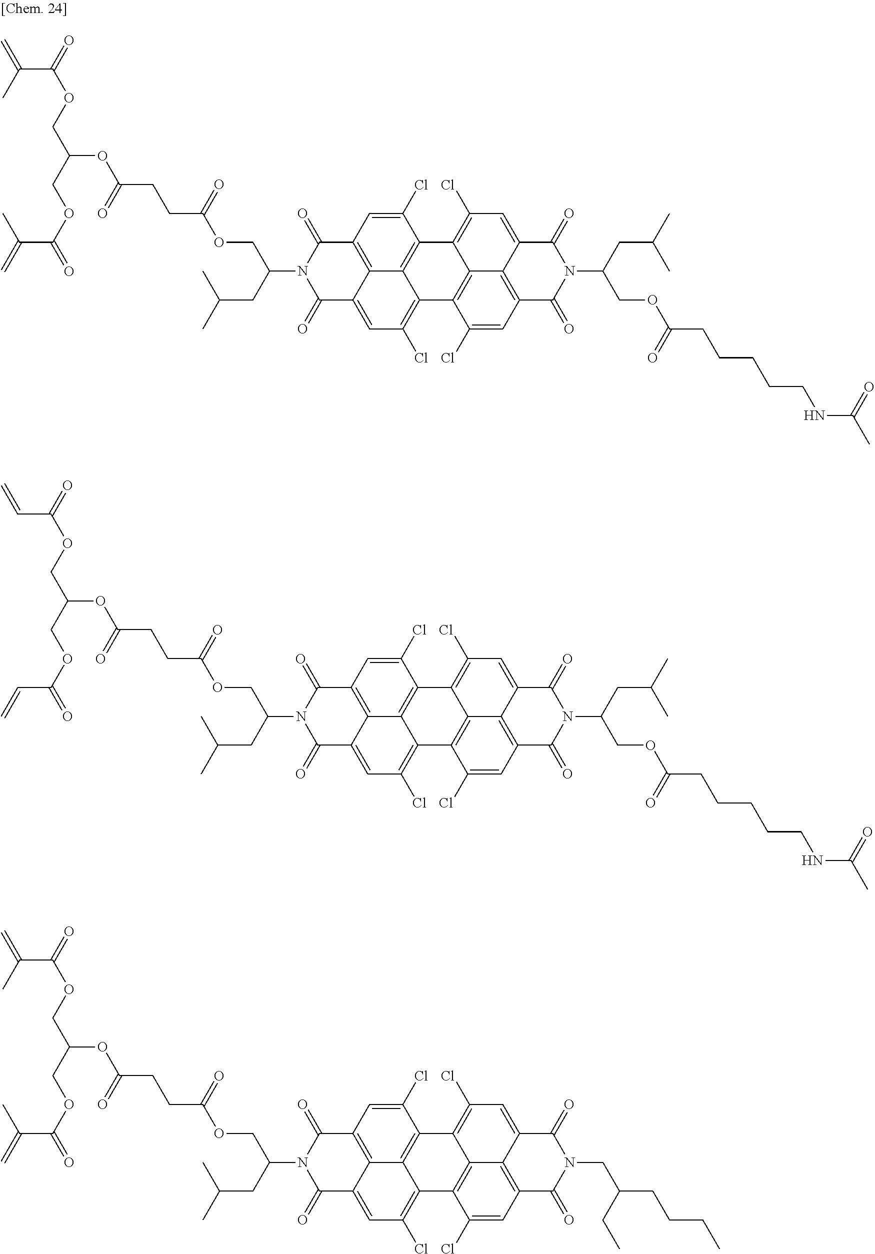 [SMILES: C=C(C)C(=O)OCC(COC(=O)C(=C)C)OC(=O)CCC(=O)OCC(CC(C)C)N1C(=O)c2cc(Cl)c3c4c(Cl)cc5c6c(cc(Cl)c(c7c(Cl)cc(c2c37)C1=O)c64)C(=O)N(C(COC(=O)CCCCCNC(C)=O)CC(C)C)C5=O ; C=CC(=O)OCC(COC(=O)C=C)OC(=O)CCC(=O)OCC(CC(C)C)N1C(=O)c2cc(Cl)c3c4c(Cl)cc5c6c(cc(Cl)c(c7c(Cl)cc(c2c37)C1=O)c64)C(=O)N(C(COC(=O)CCCCCNC(C)=O)CC(C)C)C5=O ; C=C(C)C(=O)OCC(COC(=O)C(=C)C)OC(=O)CCC(=O)OCC(CC(C)C)N1C(=O)c2cc(Cl)c3c4c(Cl)cc5c6c(cc(Cl)c(c7c(Cl)cc(c2c37)C1=O)c64)C(=O)N(CC(CC)CCCC)C5=O]

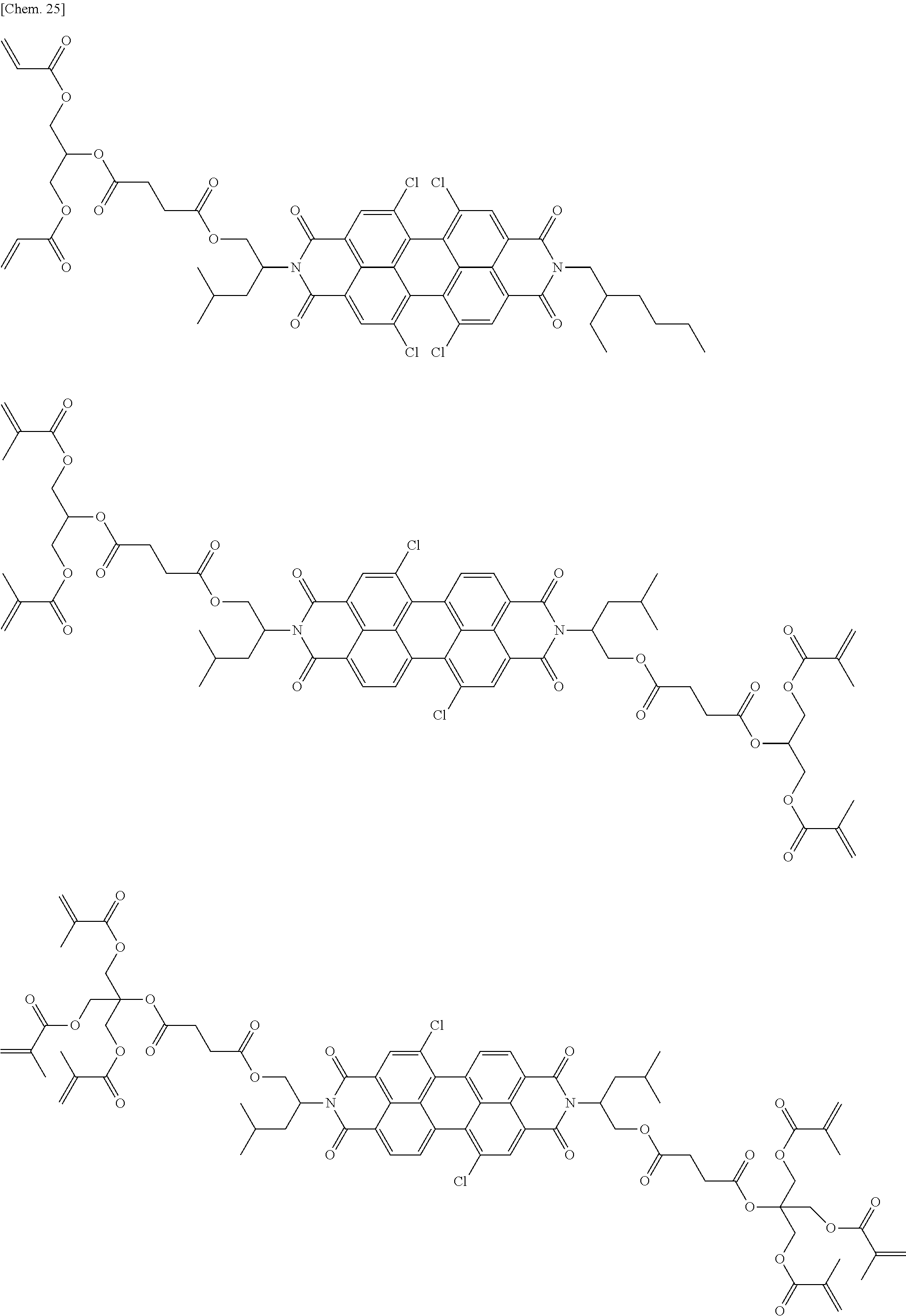

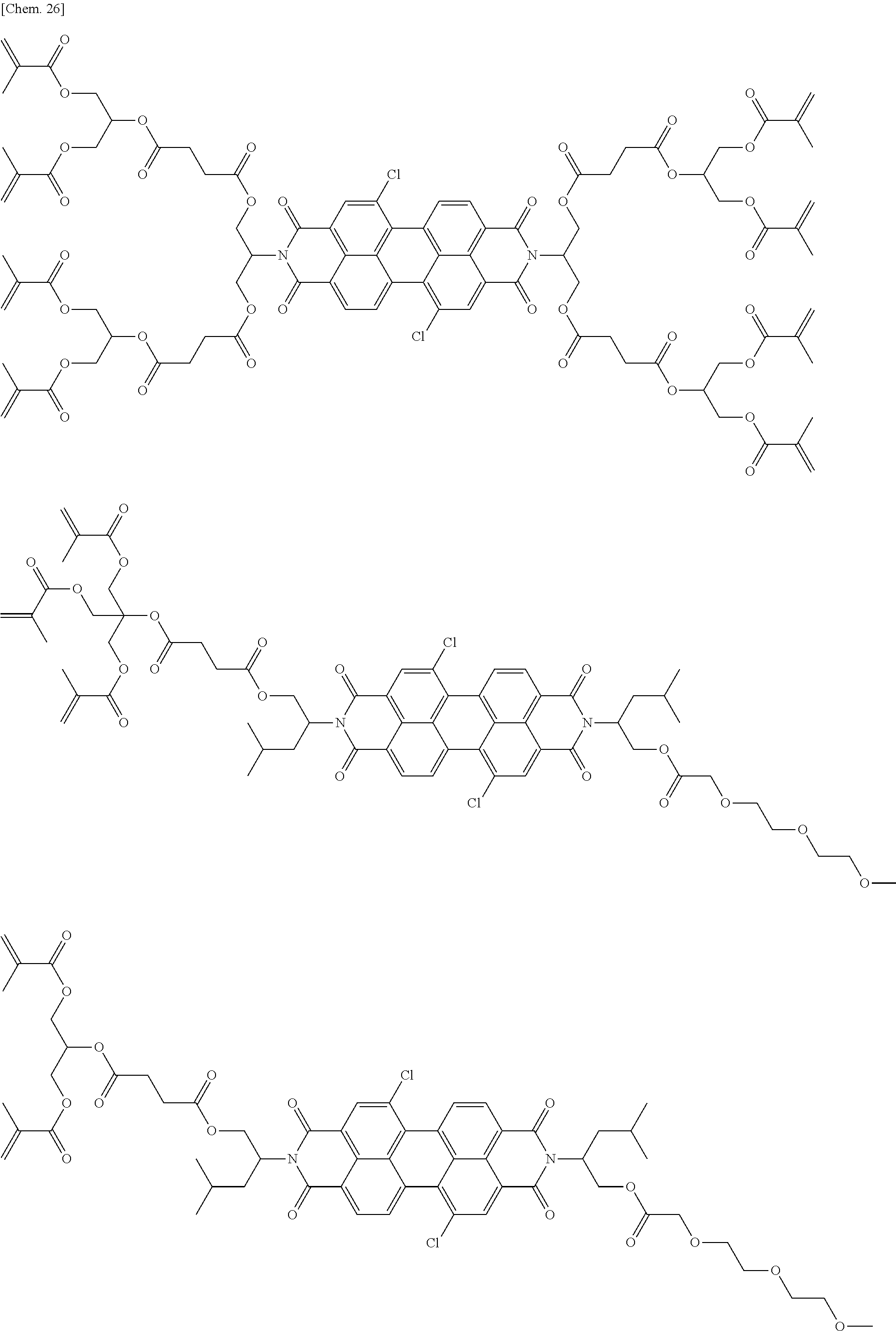

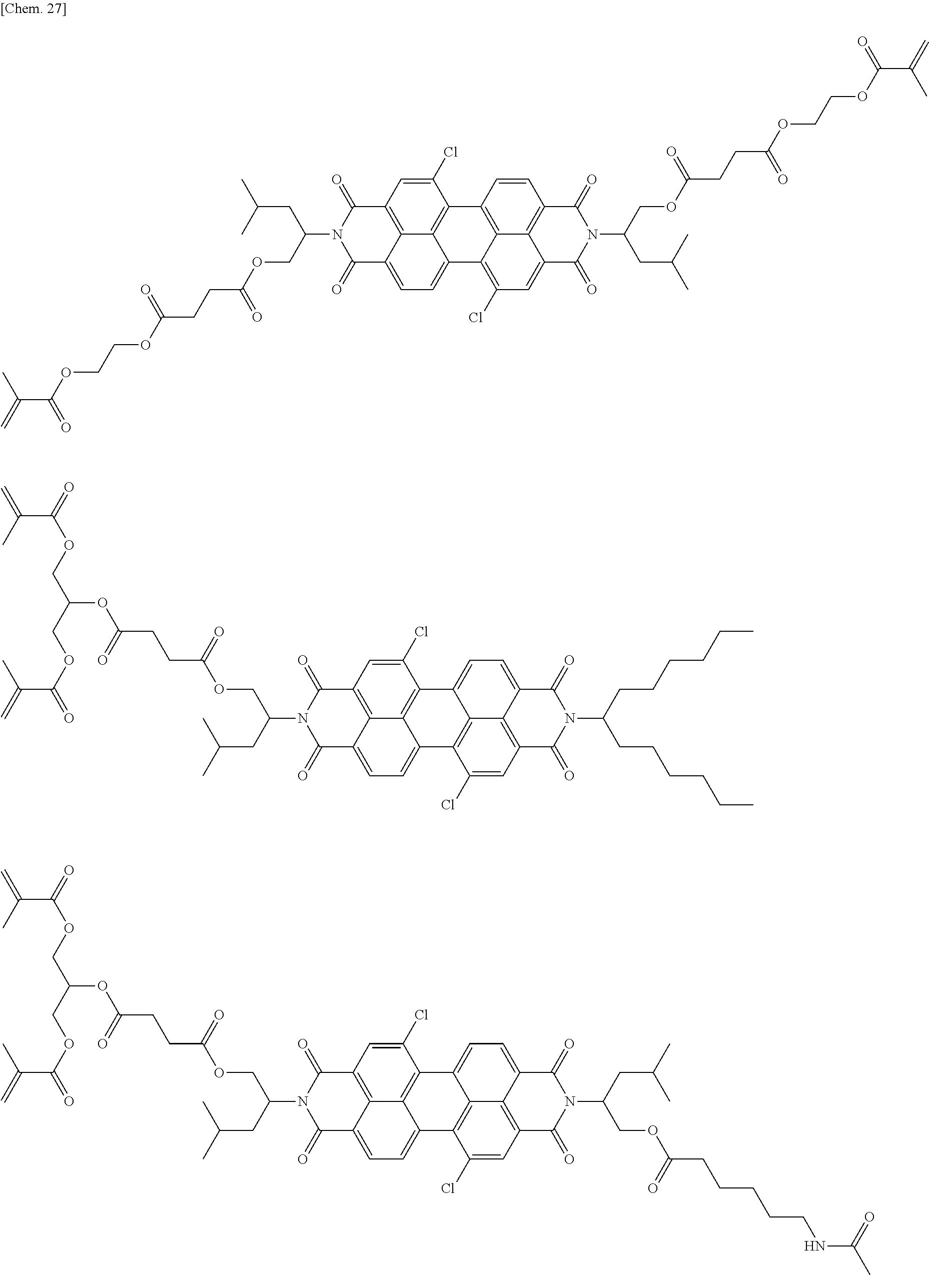

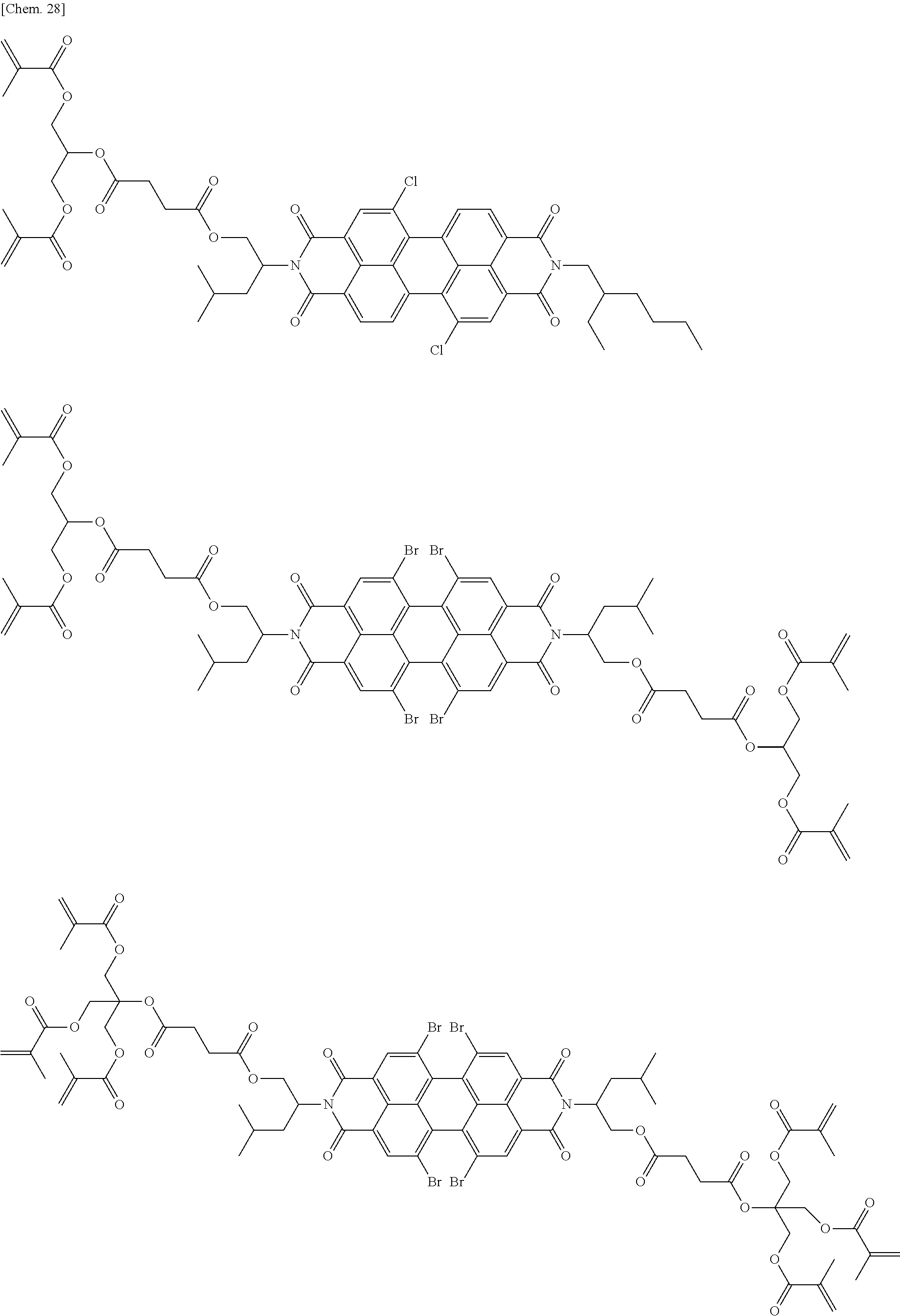

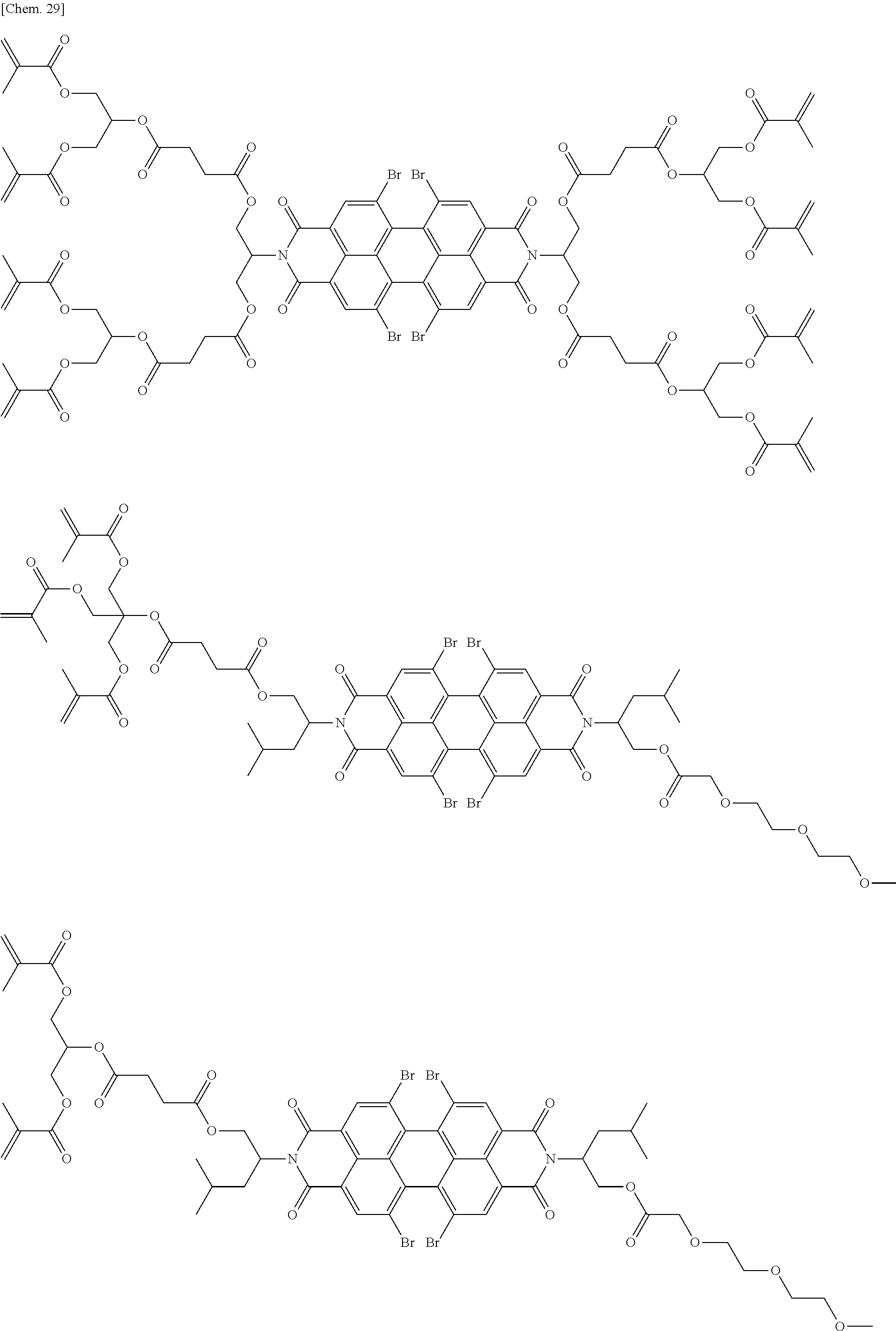

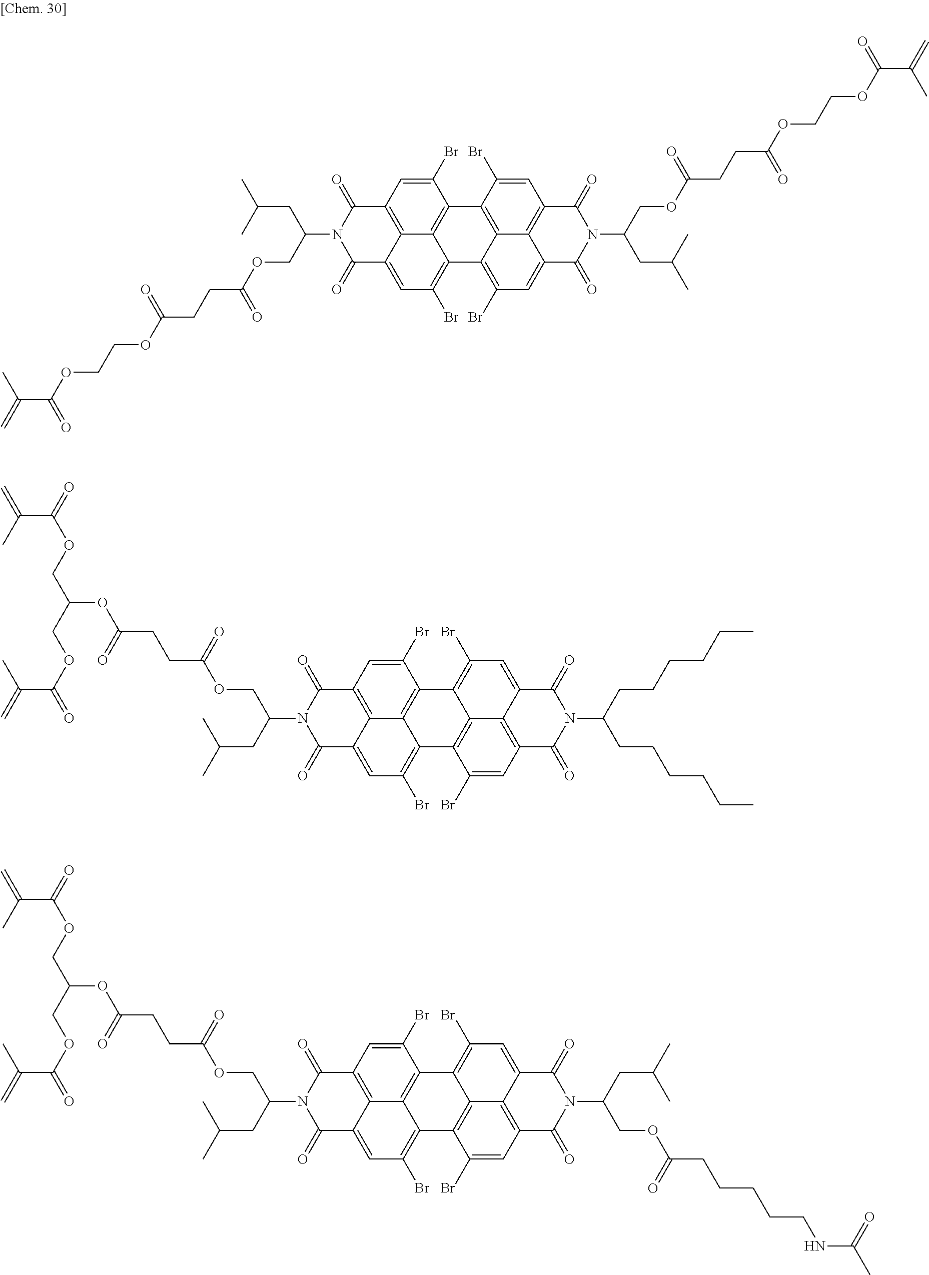

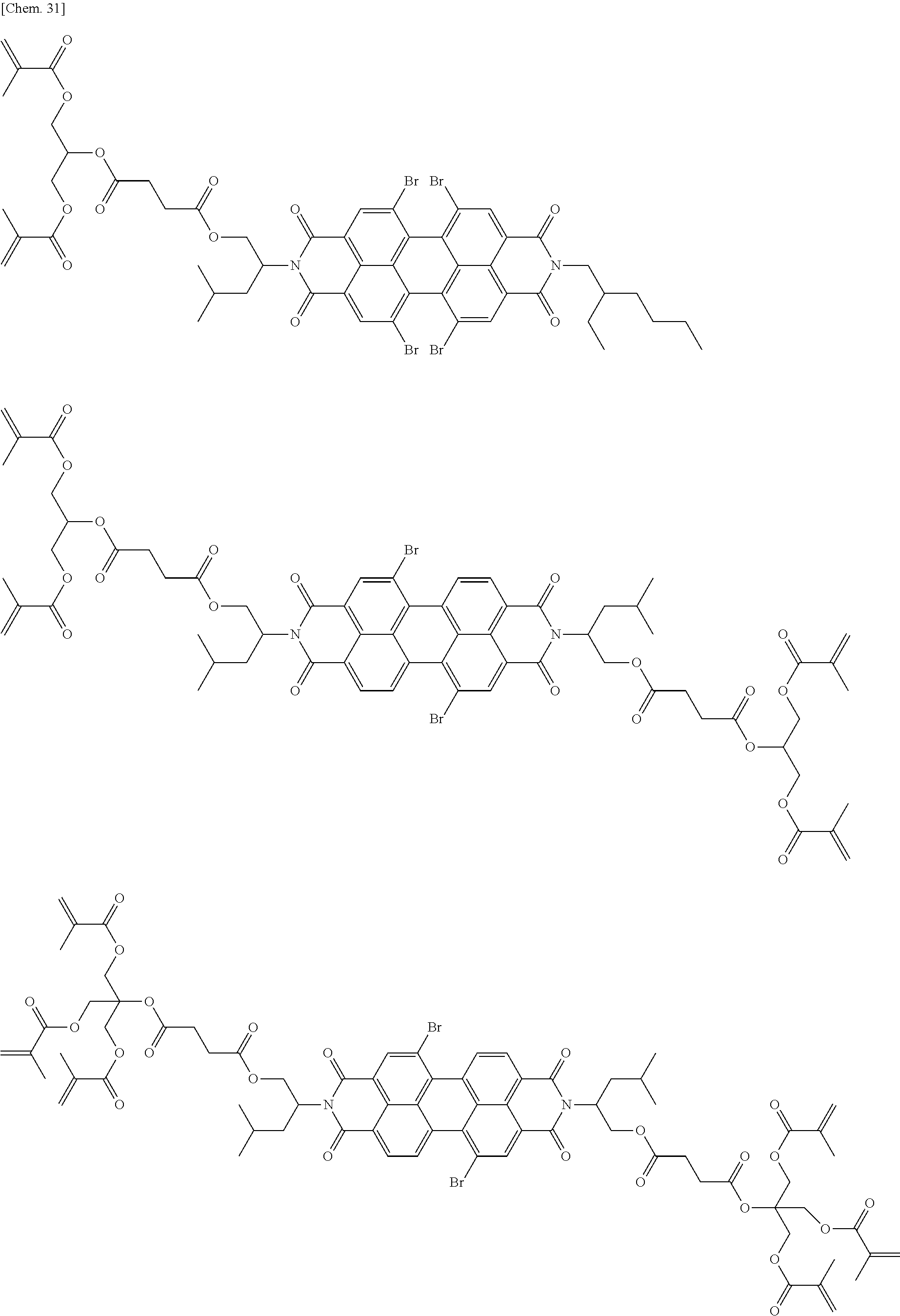

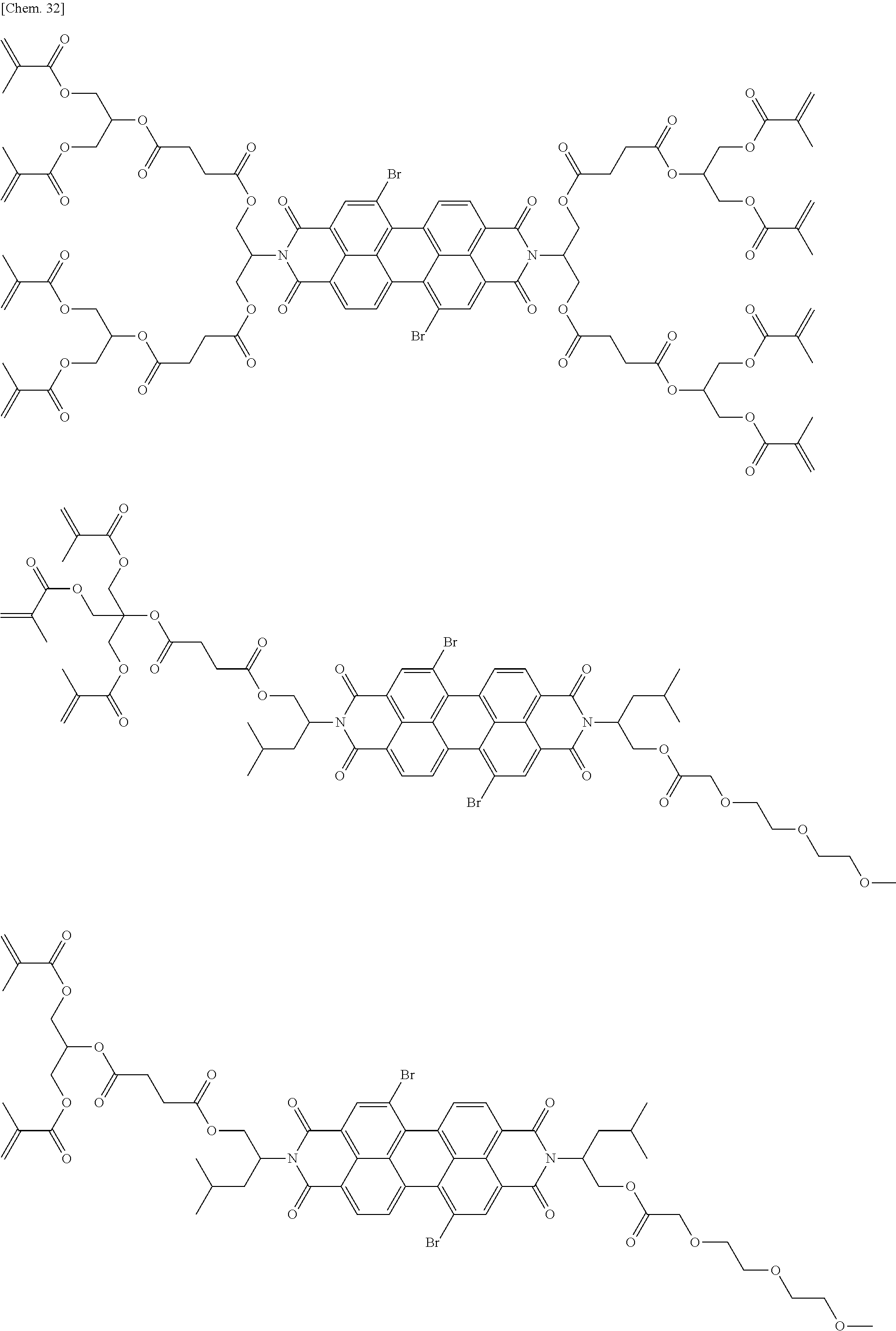

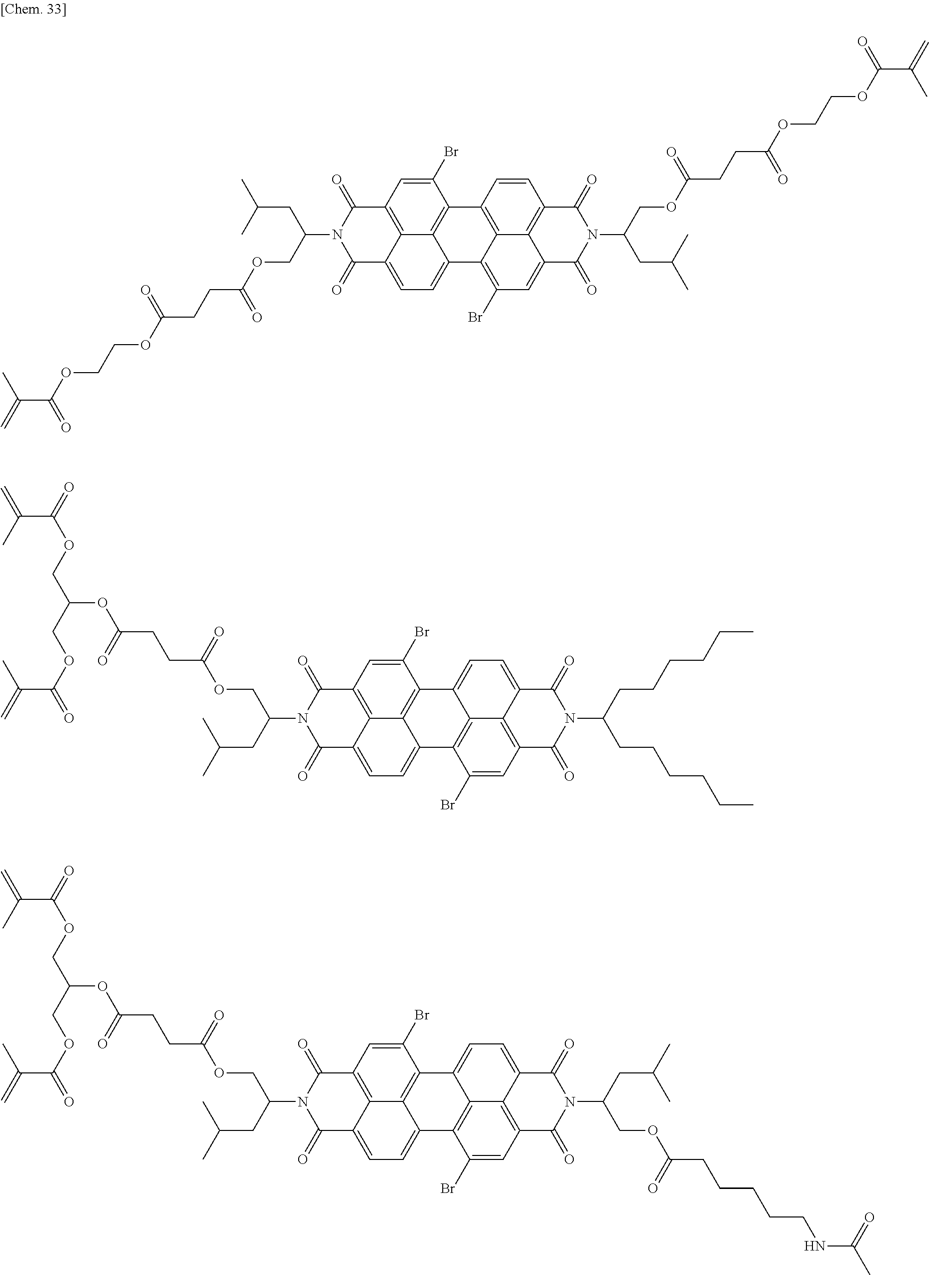

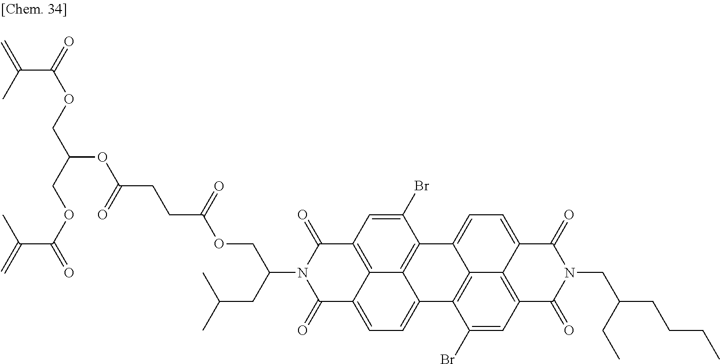 [SMILES: C=C(C)C(=O)OCC(COC(=O)C(=C)C)OC(=O)CCC(=O)OCC(CC(C)C)N1C(=O)c2ccc3c4c(Br)cc5c6c(ccc(c7c(Br)cc(c2c37)C1=O)c64)C(=O)N(CC(CC)CCCC)C5=O]

### <Method of Producing the Compound>

The compound of the present invention can be produced, for example, in accordance with the method described in the Examples below.

### <Solubility of the Compound>

The compound of the present invention has excellent solubility in organic solvents, particularly alcohol-based solvents and mixed solvents containing alcohol-based solvents, and is preferably dissolved at 3% by mass or more, particularly preferably dissolved at 6% by mass or more in a mixed solvent of toluene and 2-propanol (30% by mass of toluene and 70% by mass of 2-propanol).

### <Applications>

The compounds of the present invention are useful as protective layer forming materials for electrophotographic photoreceptors due to their excellent electron transporting property and organic solvent solubility. However, they are not limited to protective layer forming materials, and can also be used as photosensitive layer forming materials and undercoat layer forming materials for electrophotographic photoreceptors, or for applications other than materials for electrophotographic photoreceptors, such as materials for organic electroluminescent elements and materials for organic thermoelectric conversion elements.

### <<Composition>>

The composition of the present invention (hereinafter, also referred to as the “present composition”) contains the above mentioned compound of the present invention, and is particularly useful as a curable composition used in the preparation of a coating liquid for forming a protective layer for an electrophotographic photoreceptor.

Although the present composition will be described below by taking as an example a curable composition used in the preparation of a coating liquid for forming a protective layer for an electrophotographic photoreceptor, the present composition is not limited to such a curable composition.

In this embodiment, the present composition contains an electron transporting compound containing at least the compound of the present invention, and optionally contains a polymerizable compound not having an electron transporting skeleton, an electron donating compound, a polymerization initiator, inorganic particles, and other materials.

In the present invention, the term “composition” refers to a composition consisting of solid components only, without solvent.

Therefore, the content of each component, such as the compound of the present invention, and the like, based on 100 parts by mass of the composition described below corresponds to the content of each component based on 100 parts by mass of the total mass of the protective layer formed using the present composition.

The total mass of the protective layer refers to the total mass of the protective layer after curing, which is the same as the total mass of the solid components in the coating liquid for forming the protective layer described below.

### <Electron Transporting Compound>

The electron transporting compound contained in the present composition contains at least the compound of the present invention, and may contain an electron transporting compound other than the above mentioned compound of the present invention, as necessary.

The present composition may contain only one type of the compound of the present invention, or may contain two or more types.

Examples of electron transporting compounds other than the compound of the present invention include the compounds shown below.

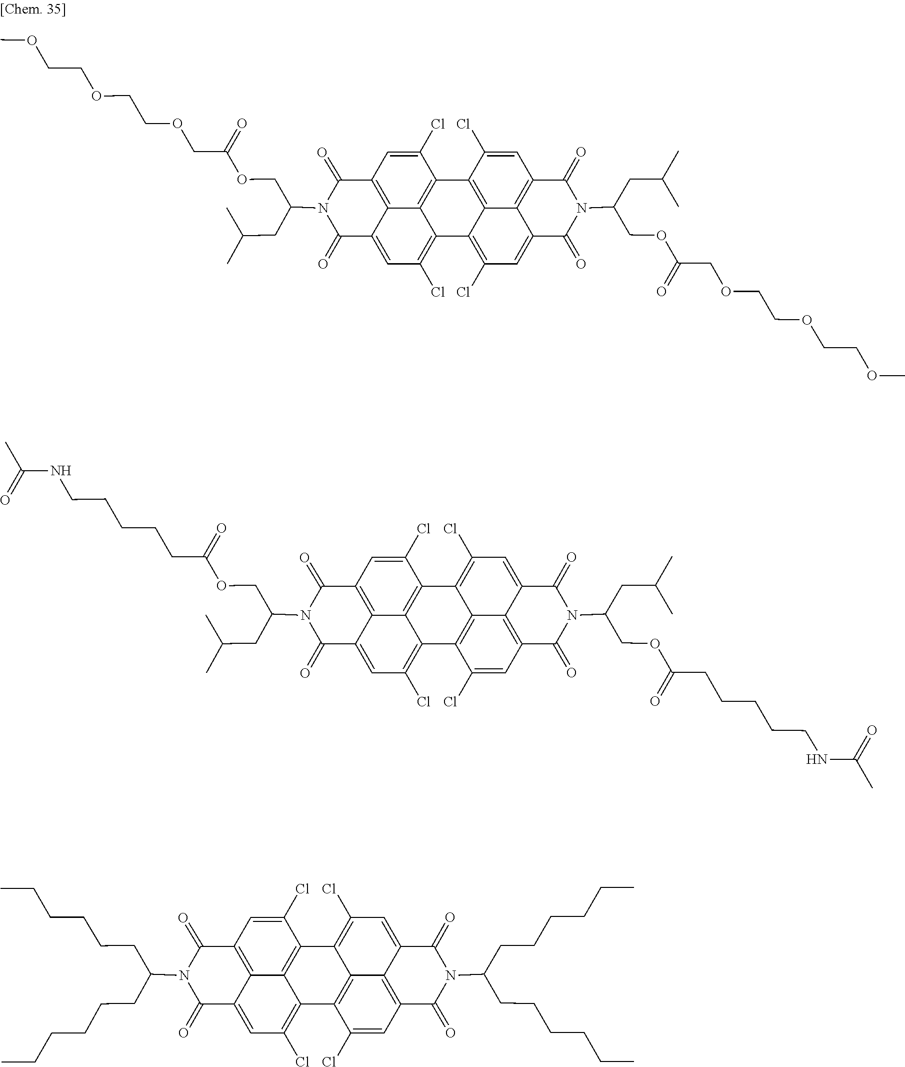

As for the electron transporting compound other than the compound of the present invention, only one type may be contained in the composition of the present invention, or two or more types may be contained.

From the viewpoint of electron transporting property, the content of the electron transporting compound in the present composition is preferably 40 parts by mass or more, more preferably 50 parts by mass or more, and even more preferably 60 parts by mass or more, based on 100 parts by mass of the total mass of the present composition. On the other hand, from the viewpoints of the hardness and elastic deformation rate of the protective layer, the content of the electron transporting compound in the present composition is preferably 90 parts by mass or less, more preferably 80 parts by mass or less, and even more preferably 70 parts by mass or less.

When the present composition contains an electron transporting compound other than the compound of the present invention, the content of the compound of the present invention is preferably 40 parts by mass or more, more preferably 50 parts by mass or more, and even more preferably 60 parts by mass or more, and may be 100 parts by mass, based on 100 parts by mass of the total mass of the electron transporting compounds in the present composition, from the viewpoint of effectively obtaining the excellent solvent solubility of the compound of the present invention.

### <Polymerizable Compound Having No Electron Transporting Skeleton>

The present composition may contain a polymerizable compound having no electron transporting skeleton.

In the case of the compound according to the first embodiment of the present invention, or in the case where the compound according to the second embodiment of the present invention has a polymerizable functional group, they can also function as a curable compound. Therefore, even if the composition using them does not contain a polymerizable compound having no electron transporting skeleton, it is possible to form a protective layer with good curability by the method described below. However, by using a polymerizable compound having no electron transporting skeleton in addition to the compound of the present invention, the mechanical strength of the protective layer formed can be more sufficiently obtained.

The polymerizable compound having no electron transporting skeleton may be any compound having a chain-polymerizable functional group. In particular, a monomer, an oligomer or a polymer having a radically polymerizable functional group is preferable. Among these, a curable compound having cross-linking property, in particular, a photocurable compound, is preferable. An example thereof is a curable compound having two or more radically polymerizable functional groups. A compound having one radically polymerizable functional group can also be used in combination.

The radically polymerizable functional group can be one of an acryloyl group (including an acryloyloxy group) and a methacryloyl group (including a methacryloyloxy group) or both groups.

Examples of the compounds which are preferable as the curable compound having a radically polymerizable functional group are shown below.

Examples of the monomer having an acryloyl group or a methacryloyl group include trimethylolpropane triacrylate (TMPTA), trimethylolpropane trimethacrylate, pentaerythritol triacrylate, pentaerythritol tetraacrylate, glycerol triacrylate, tris(acryloxyethyl)isocyanurate, dipentaerythritol hexaacrylate, dimethylolpropane tetraacrylate, pentaerythritol ethoxytetraacrylate, EO-modified phosphoric acid triacrylate, 2,2,5,5-tetrahydroxymethylcyclopentanone tetraacrylate, 2-hydroxy-3-acryloyloxy propylmethacrylate, polyethylene glycol diacrylate, polypropylene glycol diacrylate, polytetramethyleneglycol diacrylate, EO-modified bisphenol A diacrylate, PO-modified bisphenol A diacrylate, 9,9-bis[4-(2-acryloyloxyethoxy)phenyl]fluorene, tricyclodecane dimethanol diacrylate, decanediol diacrylate, hexanediol diacrylate, ethylene glycol dimethacrylate, polyethylene glycol dimethacrylate, EO-modified bisphenol A dimethacrylate, PO-modified bisphenol A dimethacrylate, tricyclodecane dimethanol dimethacrylate, decanediol dimethacrylate, hexanediol dimethacrylate, and the like.

Moreover, examples of the oligomer and the polymer having an acryloyl group or a methacryloyl group include urethane acrylate, ester acrylate, acrylic acrylate, epoxy acrylate and the like. Among these, urethane acrylate and ester acrylate are preferable, and of these, ester acrylate is more preferable.

A kind of the compounds above can be used alone, or two or more kinds thereof can be used in combination.

When the present composition contains such a polymerizable compound having no electron transporting skeleton, the content ratio (mass ratio) of the polymerizable compound to the electron transporting compound in the present composition is preferably 1.5 or less, more preferably 1.0 or less, and even more preferably 0.75 or less, from the viewpoint of electron transporting property. On the other hand, from the viewpoints of hardness and elastic deformation rate of the protective layer, this content ratio (mass ratio) is preferably 0.2 or more, more preferably 0.3 or more, and even more preferably 0.4 or more.

### <Electron Donating Compound>

The present composition may further contain an electron donating compound.

In the present invention, the term “electron donating compound” refers to a compound that can donate electrons to the protective layer. In other words, the term “electron donating compound” refers to a compound that can reduce the energy barrier (energy barrier) during electron transfer in the target compound (electron transporting compound) in the protective layer by any mechanism, and can inject electrons into the target compound. The mechanism may be, for example, a mechanism in which the electron donating compound directly transfers electrons to the target compound, a mechanism in which the electron donating compound and the target compound form hydrogen bonds to transfer electrons, or a mechanism in which the electron donating compound and the target compound form hydrogen bonds to reduce the energy barrier (energy barrier) during electron transfer, and an electron transferred from the photosensitive layer is injected into the target compound present in the protective layer.

Examples of currently known electron donating compounds include compounds having structures such as triphenylmethane, acridine, amine, amidine, aniline, pyridine, xanthene, benzimidazole, guanidine, phosphazene, and the like. Compounds that will be recognized to have such an effect in the future are also included.

As mentioned above, examples of electron donating compounds include compounds having structures such as triphenylmethane, acridine, amine, amidine, aniline, pyridine, xanthene, benzimidazole, guanidine, phosphazene, and the like. Among these, compounds having a benzimidazole structure or a guanidine structure are preferred from the viewpoint of stability. Although either a chain guanidine structure or a cyclic guanidine structure can be used as the guanidine structure, a cyclic guanidine structure is preferred from the viewpoint of stability.

As the electron donating compound, a compound having one or more heteroatoms in the molecule is preferred, and among these, a compound having one or more nitrogen atoms (N atoms) in the molecule is more preferred. From the viewpoint of stability, the number of heteroatoms in one molecule of the electron donating compound is preferably one or more, more preferably two or more, and even more preferably three or more. From the viewpoint of electron donating ability, the number of nitrogen atoms (N atoms) in one molecule of the electron donating compound is preferably one or more, more preferably two or more, and even more preferably three or more.

From the viewpoint of stability, the electron donating compound is preferably a compound having one or more cyclic structures.

The electron donating compound is preferably an electron donating compound represented by the following formula (4) or formula (5).

These electron donating compounds are activated when heated to room temperature or higher, and can donate electrons to the protective layer. Specifically, the electron donating compound represented by the following formula (4) is activated when heated to about 80° C. or higher, and can donate electrons to the protective layer. The electron donating compound represented by the following formula (5) is activated when heated to room temperature or higher, and can donate electrons to the protective layer. Thus, when forming the protective layer, these compounds are activated by the temperature rise caused by ultraviolet irradiation, and can donate electrons to the protective layer.

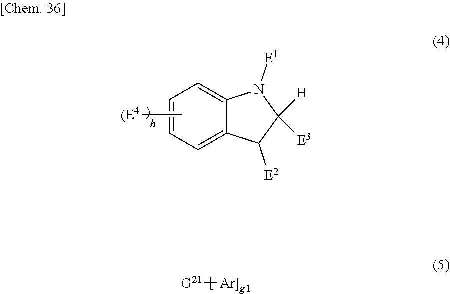

In the formula (4), E¹ to E⁴ are preferably each independently a hydrogen atom, a halogen atom, an alkyl group which may have one or more substituents, a thioalkyl group which may have one or more substituents, a thioaryl group which may have one or more substituents, an arylsulfonyl group which may have one or more substituents, an amino group which may have one or more substituents, an alkylamino group which may have one or more substituents, an arylamino group which may have one or more substituents, a hydroxy group which may have one or more substituents, an alkoxy group which may have one or more substituents, an acylamino group which may have one or more substituents, an acyloxy group which may have one or more substituents, an aromatic hydrocarbon group which may have one or more substituents, a carboxy group which may have one or more substituents, a carboxyamido group which may have one or more substituents, a carboalkoxy group which may have one or more substituents, an acyl group which may have one or more substituents, a sulfonyl group which may have one or more substituents, a cyano group which may have one or more substituents, or a nitro group which may have one or more substituents, or a derivative of any of these groups.

E¹ to E⁴ may also be bonded to each other to form a ring.

In the above formula (4), h is an integer of 0 or more, and from the viewpoint of stability, it is preferably 2 or less, more preferably 1 or less, and even more preferably 0.

In the above formula (5), g1 is an integer of 1 or more, and from the viewpoint of electrical properties, it is preferably 4 or less, more preferably 3 or less, and even more preferably 2 or less.

In the above formula (5), Ar is preferably represented by the following formula (6).

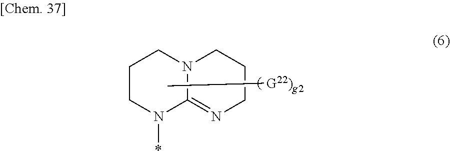

In formula (6), * represents a bond to G²¹ in the formula (5).

G²² is preferably an alkyl group which may have one or more substituents, an alkoxy group which may have one or more substituents, or a halogen atom.

In the formula (6), g2 is an integer of 0 or more, and from the viewpoint of stability, it is preferably 2 or less, more preferably 1 or less, and even more preferably 0.

In the formula (5), G²¹ is preferably a hydrocarbon group which may have one or more substituents. The number of carbon atoms in the hydrocarbon group is preferably 1 or more, more preferably 3 or more, on the other hand, it is preferably 12 or less, more preferably 10 or less. When g1 is 1, the hydrocarbon group is preferably an alkyl group, such as a methyl group, an ethyl group, a propyl group, an isopropyl group, a butyl group, a hexyl group, an octyl group, a decyl group, and the like. When g1 is 2, the hydrocarbon group is preferably an alkyl group, such as a methylene group, an ethylene group, and the like.

The content of the electron donor compound in the present composition is preferably 0.1 parts by mass or more, more preferably 0.5 parts by mass or more, and even more preferably 1.0 parts by mass or more based on 100 parts by mass of the total of the present composition, from the viewpoint of electrical properties. On the other hand, it is preferably 25 parts by mass or less, more preferably 15 parts by mass or less, and even more preferably 5.0 parts by mass or less based on 100 parts by mass of the total of the present composition, from the viewpoint of electrical properties.

Specific examples of electron donating compound are shown below. However, the electron donating compound is not limited to these.

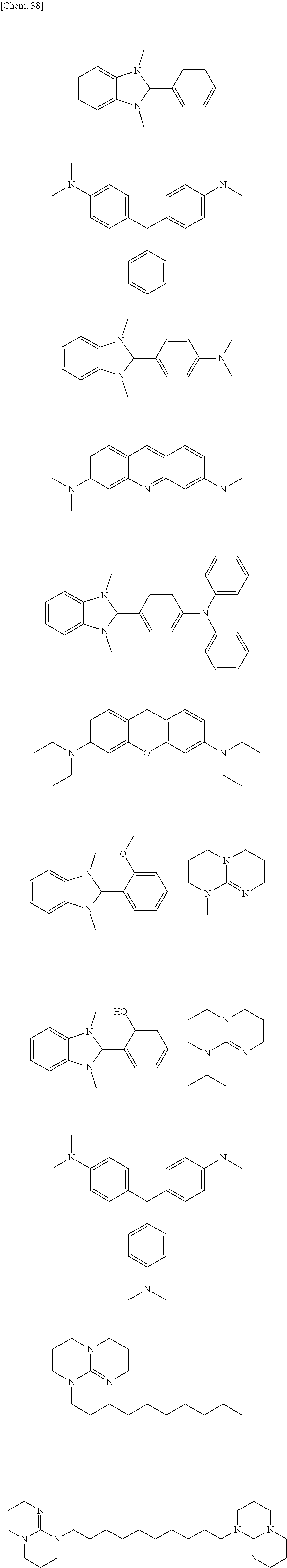

The present composition may contain only one of these electron donating compounds, or may contain two or more.

### <Polymerization Initiator>

The polymerization initiator can be a thermal polymerization initiator, a photopolymerization initiator or the like.

Examples of the thermal polymerization initiator include peroxide-based compounds such as 2,5-dimethylhexane-2,5-dihydroperoxide, and the like, and azo-based compounds such as 2,2′-azobis(isobutyronitrile), and the like.

The photopolymerization initiators can be classified into a direct cleavage type and a hydrogen extraction type based on the difference in the radical generation mechanism.

The photopolymerization initiator of the direct cleavage type generates a radical because a part of the covalent bond in the molecule is cleaved when light energy is absorbed. On the other hand, the photopolymerization initiator of the hydrogen extraction type generates a radical because the molecule in the excited state after absorbing light energy extracts hydrogen from a hydrogen donor.

Examples of the photopolymerization initiator of the direct cleavage type include acetophenone-based or ketal-based compounds such as acetophenone, 2-benzoyl-2-propanol, 1-benzoylcyclohexanol, 2,2-diethoxyacetophenone, benzyldimethylketal, 2-methyl-4′-(methylthio)-2-morpholino propiophenone, and the like, benzoin ether-based compounds such as benzoin, benzoin methylether, benzoin ethylether, benzoin isobutylether, benzoin isopropylether, O-tosylbenzoin, and the like, and acylphosphine oxide-based compounds such as diphenyl(2,4,6-trimethylbenzoyl)phosphine oxide, phenylbis(2,4,6-trimethylbenzoyl)phosphine oxide, lithium phenyl(2,4,6-trimethylbenzoyl)phosphonate, and the like.

Examples of the photopolymerization initiator of the hydrogen extraction type include benzophenone-based compounds such as benzophenone, 4-benzoylbenzoic acid, 2-benzoylbenzoic acid, methyl 2-benzoylbenzoate, methyl benzoylformate, benzyl, p-anisyl, 2-benzoylnaphthalene, 4,4′-bis(dimethylamino)benzophenone, 4,4′-dichlorobenzophenone, 1,4-dibenzoylbenzene, and the like, anthraquinone-based or thioxanthone-based compounds such as 2-ethylanthraquinone, 2-isopropylthioxanthone, 2-chlorothioxanthone, 2,4-dimethylthioxanthone, 2,4-diethylthioxanthone, 2,4-dichlorothioxanthone, and the like.

Examples of the other photopolymerization initiators include camphor quinone, 1-phenyl-1,2-propanedione-2-(o-ethoxycarbonyl)oxime, acridine-based compounds, triazine-based compounds and imidazole-based compounds.

To efficiently absorb light energy and generate a radical, the photopolymerization initiator preferably has an absorption wavelength in the wavelength range of the light source used for the light application. In particular, an acylphosphine oxide-based compound having an absorption wavelength at a relatively long wavelength side is preferably contained.

To compensate for the curability, the acylphosphine oxide-based compound and the initiator of the hydrogen extraction type are further preferably used in combination.

In this case, the ratio of the initiator of the hydrogen extraction type to the acylphosphine oxide-based compound is not particularly limited. The ratio of the initiator of the hydrogen extraction type to 1 part by mass of the acylphosphine oxide-based compound is preferably 0.1 parts by mass or more to compensate for the surface curability, and the ratio is preferably 5 parts by mass or less to maintain the internal curability.

Moreover, one having the effect of promoting photopolymerization can be used alone or in combination with the photopolymerization initiator. Examples of the one having the effect of promoting photopolymerization include triethanolamine, methyldiethanolamine, ethyl 4-dimethylaminobenzoate, isoamyl 4-dimethylaminobenzoate, (2-dimethylamino)ethyl benzoate, 4,4′-dimethylaminobenzophenone, and the like.

One kind of the polymerization initiator or a mixture of two or more kinds thereof may be used. The content of the polymerization initiator is preferably 0.5 to 40 parts by mass, and more preferably 1 to 20 parts by mass based on 100 parts by mass of the total material having radically polymerizable property.

The total material having radically polymerizable property includes the compound of the present invention and the above mentioned polymerizable compound having no electron transporting skeleton.

### <Inorganic Particles>

The present composition may contain inorganic particles from the viewpoint of improving the strong exposure property and mechanical strength of the protective layer to be formed, or from the viewpoint of imparting charge transporting capability. However, an inorganic particle is not an essential component of the composition of the present invention.

In the present invention, by using the compound of the present invention, it is possible to form a protective layer having excellent mechanical strength without containing inorganic particles.

Examples of the inorganic particles include a metal powder, a metal oxide, a metal fluoride, potassium titanate, boron nitride and the like, and any inorganic particles which can be generally used for an electrophotographic photoreceptor can be used.

As the inorganic particles, particles of one kind may be used alone, or particles of two or more kinds may also be mixed and used.

### <Other Materials>

The present composition may contain other materials other than those mentioned above according to the need. Examples of the other material include a stabilizer (a thermal stabilizer, an ultraviolet absorber, a light stabilizer, an antioxidant or the like), a dispersant, an antistatic agent, a colorant, a lubricant, and the like. One kind thereof alone or two or more kinds thereof at any ratio in any combination can be appropriately used.

### <<Electrophotographic Photoreceptor>>

An electrophotographic photoreceptor according to one embodiment of the present invention (hereinafter, also referred to as the “present electrophotographic photoreceptor”) is an electrophotographic photoreceptor having at least a photosensitive layer and a protective layer in this order on a conductive support, wherein the protective layer contains a polymer of the compound of the present invention.

The electrophotographic photoreceptor of the present invention can optionally have a layer other than the photosensitive layer and the protective layer.

Moreover, the charging method of the electrophotographic photoreceptor of the present invention may be either a negatively charging method for negatively charging the surface of the photoreceptor or a positively charging method for positively charging the surface of the photoreceptor. Of these, considering that electron transporting property is required for the protective layer, the positively charging method is preferable because the effect of the present invention is considered to be exhibited more by the positively charging method.

In the electrophotographic photoreceptor of the present invention, the side opposite to the conductive support is the upper side or the surface side, and the conductive support side is the lower side or the back surface side.

### <Protective Layer>

When the compound according to the first embodiment of the present invention is used, the protective layer of the present electrophotographic photoreceptor (hereinafter, also referred to as the “present protective layer”) contains a polymer of the compound according to the first embodiment of the present invention. The present protective layer containing the polymer of the compound according to the first embodiment of the present invention has excellent electron transporting property, and can provide a photoreceptor having excellent electrical properties, while also providing an excellent protective effect for the photoreceptor.

When the compound according to the second embodiment of the present invention is used, the present protective layer contains the compound according to the second embodiment of the present invention. By containing the compound according to the second embodiment of the present invention, the present protective layer has excellent electron transporting property.

When the compound according to the second embodiment of the present invention has a polymerizable functional group, the present protective layer using the compound according to the second embodiment of the present invention contains a polymer of the compound according to the second embodiment of the present invention. The present protective layer containing the polymer of the compound according to the second embodiment of the present invention has excellent electron transporting property, and can provide a photoreceptor having excellent electrical properties, while also providing an excellent protective effect for the photoreceptor.

Since the compound of the present invention has the above mentioned polymerizable functional group, the compound of the present invention can be polymerized in the protective layer formation process described below to become a polymer, and form a protective layer having excellent mechanical strength. In this case, the polymer may be a polymer formed by polymerizing the compounds of the present invention together, or, when a polymerizable compound having no electron transporting skeleton is contained in the protective layer, the polymer may be a copolymer formed by polymerizing the compound with the compound of the present invention.

From the viewpoint of more effectively obtaining the effect of the compound of the present invention, the present protective layer is preferably the outermost layer, that is, the outermost layer located on the opposite side to the conductive support. However, the effect of the present invention can be obtained even if the protective layer is not necessarily the outermost layer. For example, in the case where some kind of segregation layer exists on the outermost layer of the photoconductor, the effect can be obtained even if the protective layer is not the outermost layer.

The present protective layer is preferably formed by using the above mentioned present composition.

The method of forming the present protective layer using the present composition is described below.

### (Coating Liquid for Forming the Present Protective Layer)

The present protective layer can be formed by applying a coating liquid (hereinafter, also referred to as “the coating liquid for forming the present protective layer”) which is obtained by dissolving or dispersing a curable composition in a solvent or in a dispersant, on the present photosensitive layer and curing the coating liquid. The curable composition is the above mentioned present composition containing the electron transporting compound including the compound of the present invention, and containing a polymerizable compound having no electron transporting skeleton, an electron donating compound, a polymerization initiator, inorganic particles, and other materials according to the need.

The content of the electron transporting compound including the compound of the present invention in the coating liquid for forming the present protective layer is preferably 30 parts by mass or less, more preferably 20 parts by mass or less, and even more preferably 10 parts by mass or less, based on 100 parts by mass of the solvent, from the viewpoints of the film uniformity of the protective layer and solubility.

The content of the curable compounds, that is the total content of the compound of the present invention and the polymerizable compound having no electron transporting skeleton, in the coating liquid for forming the present protective layer is preferably 30 parts by mass or less, more preferably 20 parts by mass or less, and even more preferably 10 parts by mass, based on 100 parts by mass of the solvent, from the viewpoint of the residual potential. On the other hand, from the viewpoint of the hardness and elastic deformation rate of the protective layer, it is preferably 0.5 parts by mass or more, more preferably 1.0 parts by mass or more, and even more preferably 1.5 parts by mass or more.

The content of other components, that is components other than the above mentioned electron transporting compounds and the curable compounds contained in the present composition, in the coating liquid for forming the present protective layer is equivalent to the content of each component in the present composition described above.

As the solvent used for the coating liquid for forming the present protective layer, for example, an organic solvent can be used.

Examples of the organic solvent include: alcohols such as methanol, ethanol, propanol, 2-methoxyethanol, and the like; ethers such as tetrahydrofuran, 1,4-dioxane, dimethoxyethane, and the like; esters such as methyl formate, ethyl acetate, and the like; ketones such as acetone, methylethylketone, cyclohexanone, and the like; aromatic hydrocarbons such as benzene, toluene, xylene, anisole, and the like; chlorinated hydrocarbons such as dichloromethane, chloroform, 1,2-dichloroethane, 1,1,2-trichloroethane, 1,1,1-trichloroethane, tetrachloroethane, 1,2-dichloropropane, trichloroethylene, and the like; nitrogen-containing compounds such as n-butylamine, isopropanolamine, diethylamine, triethanolamine, ethylene diamine, triethylene diamine, and the like; aprotic polar solvents such as acetonitrile, N-methylpyrrolidone, N,N-dimethylformamide, dimethylsulfoxide, and the like. Any combination and any ratio of mixed solvents from among these can be used. Among these, from the viewpoints of the solubility and coating property, alcohols, ethers, aromatic hydrocarbons, and aprotic polar solvents are preferred, alcohols, ethers, and aromatic hydrocarbons are more preferred, alcohols and ethers are even more preferred, and alcohols are most preferred.

Moreover, even when the electron transporting compound used in the protective layer of the present electrophotographic photoreceptor do not dissolve in an organic solvent itself, the organic solvent can be used, for example, when the compound can dissolve in a mixed solvent with the above organic solvent. In general, coating unevenness can be reduced when a mixed solvent is used. When a dip coating method is used in the coating method described below, a solvent in which the under layer does not dissolve is preferably selected. From this point, an alcohol is particularly preferably contained.

The ratio of the solvent used for the coating liquid for forming the present protective layer and the solid content depends on the coating method of the coating liquid for forming the present protective layer and can be appropriately changed and used in such a manner that a uniform coating film is formed by the coating method applied.

### (Coating Method of the Coating Liquid for Forming the Present Protective Layer)

The method for coating the coating liquid for forming the present protective layer is not particularly limited, and examples thereof include a spray coating method, a spiral coating method, a ring coating method, a dip coating method and the like.

After forming a coating film by the coating method, the coating film is dried. In the drying process, the temperature and the period of drying are not limited so long as requiring and sufficient drying can be achieved. When the protective layer is applied after air drying alone after applying the photosensitive layer, however, sufficient drying is preferably conducted by the method described in the method for forming the photosensitive layer described below.

### (Curing Method of the Present Protective Layer)

The present protective layer can be formed by applying the coating liquid for forming the present protective layer and then curing by applying energy from outside. Examples of the external energy used here include heat, light, and radiation.

The method for applying heat energy may be a heating method using air, gas such as nitrogen, steam, various heating media, infrared ray or electromagnetic waves. Moreover, the heating can be conducted from the coated surface side or from the support side. The heating temperature is preferably 100° C. or higher and 170° C. or lower.

As the light energy, a UV light source such as a high-pressure mercury lamp, a metal halide lamp, an electrodeless lamp bulb, a light-emitting diode, and the like, which have emission wavelengths mainly of ultraviolet light (UV), can be used. Moreover, a visible light source can also be selected corresponding to the absorption wavelength of the polymerizable compound or the photopolymerization initiator.

The amount of the light irradiation is preferably 10 J/cm² or more, more preferably 15 J/cm² or more, and even more preferably 20 J/cm² or more, from the viewpoint of the curing property. Also, from the viewpoint of the electrical properties, it is preferably 400 J/cm^{z} or less, more preferably 200 J/cm^{z} or less, and even more preferably 150 J/cm² or less.

On the other hand, examples of the radiation energy include those using electron beams (EB).

Among these energies, those using light energy are preferred from the viewpoints of ease of the reaction rate control, simplicity of the device, and the long pod life.

After the protective layer is cured, a heating step may be added from the viewpoints of relieving the residual stress, relieving the residual radicals, and improving the electrical properties. The heating temperature is preferably 60° C. or higher, and more preferably 100° C. or higher, and is preferably 200° C. or lower, and more preferably 150° C. or lower.

### (Layer Thickness)

The thickness of the present protective layer is preferably 0.5 μm or more, and more preferably 1 μm or more from the viewpoint of the abrasion resistance. On the other hand, from the viewpoint of the electrical properties, it is preferably 5 μm or less, and more preferably 4 μm or less.

Also, from the same viewpoint, the thickness of the present protective layer is preferably 1/50 or more of the thickness of the photosensitive layer, more preferably 1/40 or more, and even more preferably 1/30 or more. On the other hand, it is preferably ⅕ or less, more preferably 1/10 or less, and even more preferably 1/20 or less.

### <Photosensitive Layer>

The photosensitive layer (hereinafter, also referred to as “the present photosensitive layer”) in the present electrophotographic photoreceptor may be a layer containing at least a charge generating material (CGM) and a charge transporting material.

The present photosensitive layer may be a single layer type photosensitive layer containing both a charge generating material and a charge transporting material in the same layer, or may be a laminate type photosensitive layer which is separated into a charge generation layer and a charge transport layer.

### <Single Layer Type Photosensitive Layer>

When the present photosensitive layer is a single layer type photosensitive layer, it is preferable that at least a charge generating material (CGM), a hole transporting material (HTM), an electron transporting material (ETM), and a binder resin are contained in the same layer.

### (Charge Generating Material)

Examples of the charge generating material (CGM) used in the present photosensitive layer include various photoconductive materials such as inorganic photoconductive materials and organic pigments, or the like. Among these, organic pigments are particularly preferable, and phthalocyanine pigments and azo pigments are more preferable.

In particular, when a phthalocyanine pigment is used as the charge generating material, titanyl phthalocyanine of A type, B type, D type, and the like, chlorogallium phthalocyanine, hydroxygallium phthalocyanine, and the like are suitable.

When an azo pigment is used, various known bisazo pigments and trisazo pigments are suitably used.

One kind of the charge generating material may be used alone, or two or more kinds thereof may be used at any ratio in any combination.

Furthermore, from the viewpoint of the sensitivity, the amount of the charge generating material in the single layer type photosensitive layer is preferably 0.1% by mass or more, and more preferably 0.5% by mass or more. From the viewpoints of the sensitivity and the electrostatic property, it is preferably 50% by mass or less, and more preferably 20% by mass or less.

### (Charge Transporting Material)

Charge transporting materials are classified into hole transporting materials that mainly have hole transporting ability and electron transporting materials that mainly have electron transporting ability. However, when the present photosensitive layer is a single layer type photosensitive layer, it is preferable to contain at least a hole transporting material and an electron transporting material in the same layer.

### (Hole Transporting Material)

The hole transporting material (HTM) can be selected from known materials and used. Examples thereof include electron donating materials, such as heterocyclic compounds including a carbazole derivative, an indole derivative, an imidazole derivative, an oxazole derivative, a pyrazole derivative, a thiadiazole derivative, a benzofuran derivative and the like, an aniline derivative, a hydrazone derivative, an arylamine derivative, a stilbene derivative, a butadiene derivative, an enamine derivative, and a material in which two or more kinds of the compounds are bound, and a polymer having a group derived from such a compound in the main chain or a side chain, and the like.

Among these, a carbazole derivative, an arylamine derivative, a stilbene derivative, a butadiene derivative, an enamine derivative and a material in which two or more kinds of the compounds are bound are preferable, and an arylamine derivative and an enamine derivative are more preferable.

One kind of the hole transporting material may be used alone, or two or more kinds thereof may be used at any ratio in any combination.

The amount of the hole transporting material in the single layer type photosensitive layer is preferably 20% by mass or more, and more preferably 30% by mass or more based on 100% by mass of the total of the present photosensitive layer, from the viewpoint of the hole transporting property.

From the viewpoint of the solubility, it is preferably 55% by mass or less, and more preferably 45% by mass or less.

### (Electron Transporting Material)

The electron transporting material (ETM) can be selected from known materials and used. Examples thereof include electron withdrawing materials including aromatic nitro compounds such as 2,4,7-trinitrofluorenone, and the like, cyano compounds such as tetracyanoquinodimethane, and the like, quinone compounds such as diphenoquinone, and the like, known cyclic ketone compounds, perylene pigments (perylene derivatives) and the like. Among these, from the viewpoint of the electrical properties, quinone compounds and perylene pigments (perylene derivatives) are preferred, and quinone compounds are more preferred.

Among the quinone compounds, diphenoquinone or dinaphthylquinone is preferred from the viewpoint of the electrical properties. Among them, dinaphthylquinone is more preferred.

One kind of the electron transporting material may be used alone, or two or more kinds thereof may be used at any ratio in any combination.

The amount of the electron transporting material in the single layer type photosensitive layer is preferably 15% by mass or more, and more preferably 25′ by mass or more, based on 100% by mass of the total of the present photosensitive layer, from the viewpoint of the electron transporting property. From the viewpoint of the solubility, it is preferably 40% by mass or less, and more preferably 30% by mass or less.

### (Binder Resin)

Examples of the binder resin used for the present photosensitive layer include: vinyl polymers such as polymethylmethacrylate, polystyrene, polyvinyl chloride, and the like or copolymers thereof; vinyl alcohol resins; polyvinyl butyral resins; polyvinyl formal resins; partially modified polyvinyl acetal resins; polyarylate resins; polyamide resins; polyurethane resins; polycarbonate resins; polyester resins; polyester carbonate resins; polyimide resins; phenoxy resins; epoxy resins; silicone resins; and partially cross-linked cured materials thereof. The resins may be modified with a silicon reagent or the like. One kind thereof may be used alone, or two or more kinds thereof can be used at any ratio in any combination.

### (Other Substances)

In addition to the above materials, the present photosensitive layer may contain a known additive such as an antioxidant, a plasticizer, an ultraviolet absorber, an electron-withdrawing compound, a leveling agent, a visible light-shielding agent, and the like to improve the film forming property, the flexibility, the coatability, the contamination resistance, the gas resistance, the light resistance or the like. Moreover, the present photosensitive layer may contain various additives such as a sensitizer, a dye, a pigment (excluding those which are the charge generating material, the hole transporting material and the electron transporting material listed above), a surfactant, and the like according to the need. Examples of the surfactant include silicone oil, a fluorine-based compound and the like. In the present invention, one kind thereof alone or two or more kinds thereof at any ratio in any combination can be appropriately used.

Moreover, for the purpose of reducing the friction resistance on the surface of the photosensitive layer, the present photosensitive layer may contain a fluorine-based resin, a silicone resin or the like or may contain particles of such a resin or particles of an inorganic compound such as aluminum oxide.

### (Layer Thickness)

When the present photosensitive layer is a single layer type photosensitive layer, the thickness of the present photosensitive layer is preferably 20 μm or more, and more preferably 25 μm or more, from the viewpoint of the dielectric breakdown resistance. On the other hand, from the viewpoint of the electrical properties, the thickness is preferably 50 μm or less, and more preferably 40 μm or less.

### <Laminate Type Photosensitive Layer>

When the present photosensitive layer is a laminate type photosensitive layer, the photosensitive layer can have a configuration obtained, for example, by laminating a charge transport layer (CTL) containing a charge transporting material on a charge generation layer (CGL) containing a charge generating material (CGM). At this point, a layer other than the charge generation layer (CGL) and the charge transport layer (CTL) can also be included.

### (Charge Generation Layer (CGL))

The charge generation layer (CGL) usually contains a charge generating material (CGM) and a binder resin.

The charge generating material (CGM) and the binder resin are the same as those described for the single layer type photosensitive layer above.

The charge generation layer can contain another component according to the need in addition to the charge generating material and the binder resin. For example, a known additive such as an antioxidant, a plasticizer, an ultraviolet absorber, an electron-withdrawing compound, a leveling agent, a visible light-shielding agent, a filler, and the like may be contained.

When the ratio of the charge generating material in the charge generation layer is too high, the stability of the coating liquid may decrease due to aggregation of the charge generating material or the like, while when the ratio of the charge generating material is too low, the sensitivity as the photoreceptor may decrease. Therefore, the compounding ratio (mass) of the charge generating material is preferably 10 parts by mass or more, and more preferably 30 parts by mass or more, based on 100 parts by mass of the binder resin. On the other hand, the compounding ratio (mass) of the charge generating material is preferably 1000 parts by mass or less, and more preferably 500 parts by mass or less, based on 100 parts by mass of the binder resin. From the viewpoint of the film the strength, it is even more preferable that the ratio is 300 parts by mass or less, and especially preferably 200 parts by mass or less.

The thickness of the charge generation layer is preferably 0.1 μm or more, and more preferably 0.15 μm or more. On the other hand, it is preferably 10 μm or less, and more preferably 0.6 μm or less.

### (Charge Transport Layer (CTL))

The charge transport layer (CTL) usually contains a charge transporting material and a binder resin.

The charge transporting material and binder resin are the same as those described for the single layer type photosensitive layer above.

In the charge transport layer (CTL), the compounding ratio of the charge transporting material to the binder resin is preferably 20 parts by mass or more based on 100 parts by mass of the binder resin, more preferably 30 parts by mass or more from the viewpoint of reducing the residual potential, and even more preferably 40 parts by mass or more from the viewpoints of the stability during repeated use and the charge transfer degree. On the other hand, from the viewpoint of the thermal stability of the photosensitive layer, the charge transporting material is preferably blended at a ratio of 200 parts by mass or less to 100 parts by mass of the binder resin, and from the viewpoint of the compatibility between the charge transporting material and the binder resin, the charge transporting material is more preferably blended at a ratio of 150 parts by mass or less, and from the viewpoint of the glass transition temperature, the charge transporting material is particularly preferably blended at a ratio of 120 parts by mass or less.

The charge transport layer may contain other components according to the need in addition to the charge transporting material and the binder resin. For example, it may contain a known additive such as an antioxidant, a plasticizer, an ultraviolet absorber, an electron-withdrawing compound, a leveling agent, a visible light-shielding agent, a filler, and the like.

The thickness of the charge transport layer is not particularly limited. From the viewpoints of the electrical properties and the image stability, and furthermore, from the viewpoint of the high resolution, the thickness is preferably 5 μm or more and 50 μm or less, more preferably 10 μm or more and 40 μm or less, and even more preferably 15 μm or more and 35 μm or less.

### <Method of Forming the Photosensitive Layer>

In both the laminate type and the single layer type, each of the above layers can be formed as follows.

Each layer can be formed by a coating step of coating a liquid to form a coated layer on a conductive support and a drying step drying the coated layer. The coating liquid is prepared by dissolving or dispersing the materials to be contained in a solvent. The coating step is performed by a known method such as dip coating, spray coating, nozzle coating, bar coating, roll coating, blade coating, and the like.

However, the forming method is not limited to such a method.

There is no particular restriction on the solvent or dispersion medium used to prepare the coating liquid. Specific examples include alcohols, ethers, aromatic hydrocarbons, chlorinated hydrocarbons, and the like. One kind thereof may be used alone, or two or more kinds thereof in any combination of any kinds may be used in combination.

The amount of the solvent or the dispersion medium used is not particularly restricted. Considering the purposes of the layers and the properties of the solvent/dispersion medium selected, the amount is preferably appropriately adjusted in such a manner that the solid concentrations of the coating liquids and the physical properties such as viscosity are in the desired ranges.

The coating films are preferably dried by drying to the touch at room temperature and then heat drying usually in a temperature range of 30° C. or higher and 200° C. or lower for 1 minute to 2 hours in still state or under airflow. The heating temperature may be constant, or heating may be conducted while changing the temperature during drying.

### <Conductive Support>

The conductive support (hereinafter, also referred to as “the present conductive support”) of the present electrophotographic photoreceptor is not particularly limited as long as it supports the layers formed thereon and exhibits electrical conductivity.

Examples of the present conductive support include a metal material such as aluminum, an aluminum alloy, stainless steel, copper, nickel, and the like; a resin material to which conductivity is imparted by containing conductive powder such as metal, carbon, tin oxide, and the like; a resin material, glass material or paper material having a surface on which a conductive material such as aluminum, nickel, ITO (indium oxide-tin oxide alloy) is deposited or applied.

Examples of the form of the present conductive support include a drum, a cylinder, a sheet and a belt or the like.

The present conductive support may be obtained by applying a conductive material having appropriate resistance on a conductive support made of metal material to regulate the conductivity, the surface property or the like or to cover the defect.

When a metal material such as an aluminum alloy is used as the present conductive support, the metal material may be coated with an anodized film.

The surface of the present conductive support may be smooth or may be roughened using a special cutting method or by subjecting to grinding. Moreover, the surface may be roughened by mixing particles having an appropriate particle size in the material composing the support.

### <Present Undercoat Layer>

The present electrophotographic photoreceptor may have an undercoat layer (hereinafter, also referred to as “the present undercoat layer”) between the present conductive support and the present photosensitive layer in order to improve the adhesion, the blocking property, or the like.

Examples of the present undercoat layer include a resin and a material obtained by dispersing particles of an organic pigment, a metal oxide or the like in a resin. The present undercoat layer may also contain a known antioxidant, and the like.

Examples of the organic pigment used for the undercoat layer include a phthalocyanine pigment, an azo pigment, a perylene pigment and the like. Among these, a phthalocyanine pigment or an azo pigment, specifically, the phthalocyanine pigment and the azo pigment used as the charge generating material described above, can be used.

Examples of the metal oxide particles used for the present undercoat layer include metal oxide particles containing one kind of metal element such as titanium oxide, aluminum oxide, silicon oxide, zirconium oxide, zinc oxide, iron oxide, and the like and metal oxide particles containing two or more metal elements such as calcium titanate, strontium titanate, barium titanate, and the like. One kind of the particles may be used alone for the undercoat layer, or more than one kind of the particles at any ratio in any combination may be mixed and used.

Examples of the binder resin for the present undercoat layer include a polyvinyl acetal-based resins such as a polyvinyl butyral resin, and the like; an insulating resins such as a polyarylate resin, a polycarbonate resin, a polyester resin, a phenoxy resin, an acrylic resin, a methacrylic resin, a polyamide resin, a polyurethane resin, an epoxy resin, a silicone resin, a polyvinyl alcohol resin, a styrene-alkyd resin, and the like; and the like. In this regard, however, the binder resin is not limited to these polymers. One kind of the binder resins may be used alone, or two or more kinds thereof may be mixed and used. The binder resin may be used in the form cured with a curing agent.

The thickness of the present undercoat layer can be selected freely. From the characteristics of the electrophotographic photoreceptor and the coatability of the above mentioned dispersion, the thickness is preferably 0.1 μm or more and is further preferably 20 μm or less.

### <Other Layers>

The present electrophotographic photoreceptor may have other layers according to the need in addition to the present conductive support, the present photosensitive layer, the present protective layer, and the present undercoat layer described above.

### <Residual Potential Property>

In the present electrophotographic photoreceptor, the residual potential property is preferably 200V or less, more preferably 150V or less, and even more preferably 100V or less, from the viewpoint of providing practically sufficient residual potential property.

In the present invention, the residual potential of the photoreceptor means the potential after the photoreceptor is charged and irradiated with exposure light.

The residual potential can be measured by the method described in the Examples below.

### <Potential Retention Rate>

Also in the present electrophotographic photoreceptor, the potential retention rate is preferably 60% or more, more preferably 70% or more, and even more preferably 80% or more, from the viewpoint of providing practically sufficient potential retention rate.

In the present invention, the potential retention rate (dark decay, DDR) of the photoreceptor means the surface potential retention rate (%) when the photoreceptor having a charged surface is left for a certain period of time.

The potential retention rate can be measured by the method described in the Examples below.

### <<Present Image Formation Device>>

Using the present electrophotographic photoreceptor, an image formation device (hereinafter, also referred to “the present image forming device”) can be configured.

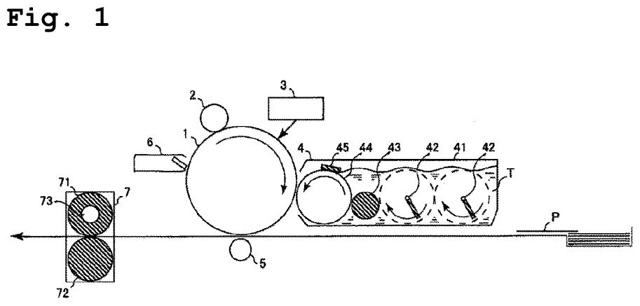

As illustrated in FIG. 1, the present image formation device is configured with the present electrophotographic photoreceptor 1, a charging device 2, an exposure device 3 and a developing device 4, and according to the need, a transfer device 5, a cleaning device 6 and a fixing device 7 are further provided.

The present electrophotographic photoreceptor 1 is not particularly restricted as long as it is the present electrophotographic photoreceptor of described above. As an example thereof, FIG. 1 illustrates a drum-type photoreceptor in which the photosensitive layer described above is formed on the surface of a cylindrical conductive support. The charging device 2, the exposure device 3, the developing device 4, the transfer device 5 and the cleaning device 6 are arranged along the peripheral surface of the present electrophotographic photoreceptor 1.

The charging device 2 can be a non-contact corona charging device such as a corotron and a scorotron or a contact-type charging device (a direct charging device) for charging by bringing a charged material to which voltage is applied into contact with the photoreceptor surface. Examples of the contact charging device include a charging roller, a charging brush and the like. In FIG. 1, a roller-type charging device (charging roller) is illustrated as an example of the charging device 2.

The type of the exposure device 3 is not particularly restricted as long as it can expose the present electrophotographic photoreceptor 1 and form an electrostatic latent image on the photosensitive surface of the present electrophotographic photoreceptor 1.

Moreover, exposure may be conducted by a photoreceptor internal exposure method. The light for the exposure may be any light.

The type of a toner T may be any type, and in addition to a powdery toner, a polymerized toner using a suspension polymerization method, an emulsion polymerization method or the like and the like can be used.

The type of the transfer device 5 is not particularly restricted, and a device using any type such as an electrostatic image transfer method including corona transfer, roller transfer, belt transfer and the like, a pressure transfer method and an adhesion transfer method can be used.

The cleaning device 6 is not particularly restricted. For example, any cleaning device such as a brush cleaner, a magnetic roller cleaner and a blade cleaner can be used. When a small amount of the toner or almost no toner remains on the surface of the photoreceptor, the cleaning device 6 does not have to be provided.

Here, the image formation device may have a configuration which can conduct, for example, a charge elimination step in addition to the configuration described above.

Moreover, the image formation device may be configured with further modification and may have, for example, a configuration which can conduct a step such as a pre-exposure step and a supplemental charging step, a configuration for conducting offset printing or a configuration of the full-color tandem type using more than one kind of toner.

### <<Present Electrophotographic Cartridge>>

The present electrophotographic photoreceptor 1 can be configured as an integrated cartridge (hereinafter, also referred to as “the present electrophotographic cartridge”) by combining with one or two or more of the charging device 2, the exposure device 3, the developing device 4, the transfer device 5, the cleaning device 6 and the fixing device 7.

The present electrophotographic cartridge can have a configuration which can be attached to and detached from the main body of an electrophotographic device such as a copier and a laser beam printer. In this case, for example, when the present electrophotographic photoreceptor 1 or another member is deteriorated, by removing the electrophotographic photoreceptor cartridge from the main body of the image formation device and installing a new electrophotographic photoreceptor cartridge into the main body of the image formation device, maintenance and the management of the image formation device become easy.

### Explanation of Terms

In the present invention, the expression “X to Y” (X and Y are numbers) includes the meaning of “X or more and Y or less” and the meaning of “preferably larger than X” or “preferably smaller than Y” unless otherwise specified. Moreover, the expression “X or more” (X is a number) or “Y or less” (Y is a number) also includes the meaning of “preferably larger than X” or “preferably less than Y”.

### Examples

Embodiments of the present invention will be explained further specifically with Examples below.

The following Examples are for explaining the present invention in detail, and the present invention is not limited to the Examples shown below and can be carried out with any modification unless departing from the gist thereof.

The “parts” in the Examples and the Comparative Examples below are “parts by mass” unless otherwise specified.

Abbreviations are as follows.
MEHQ: 4-methoxyphenol
NMP: N-methylpyrrolidone

### [Synthesis of Compounds]

The synthesis methods of Compounds 1 and 2, which are the compounds of the present invention, and Comparative Compounds 1 to 5, which are the compounds for comparison, are described below.

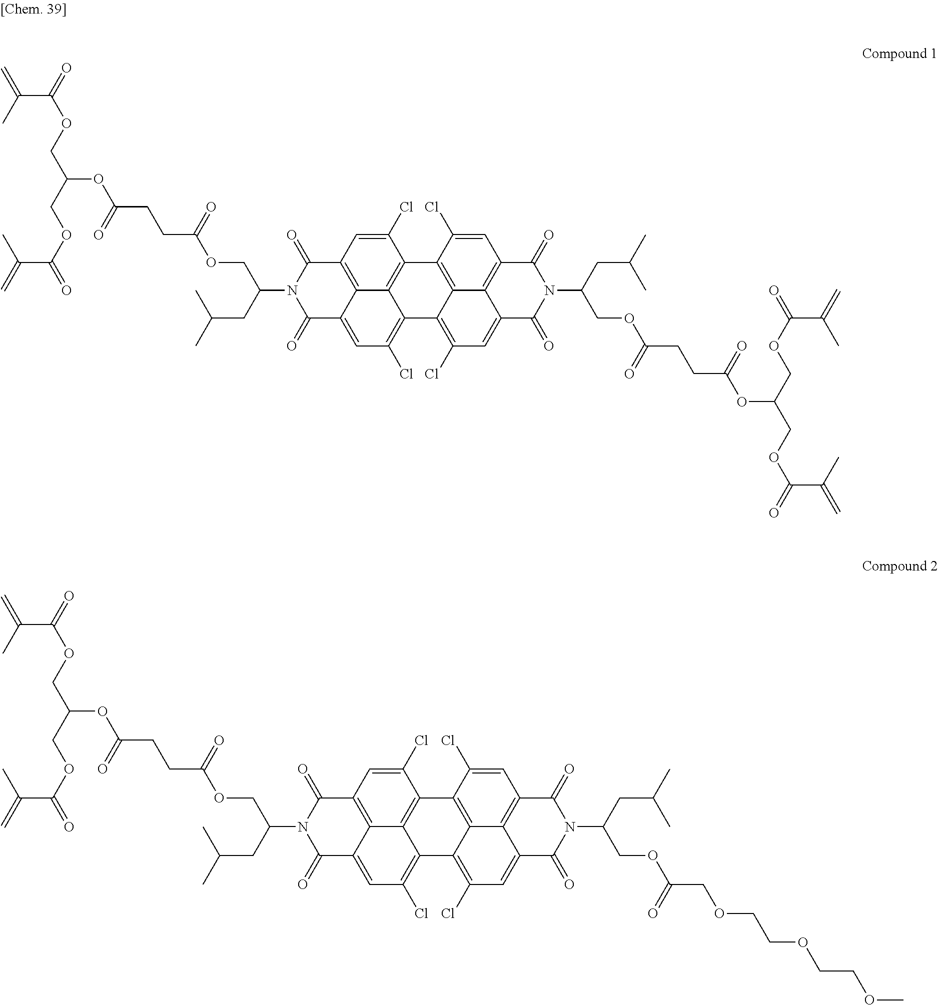

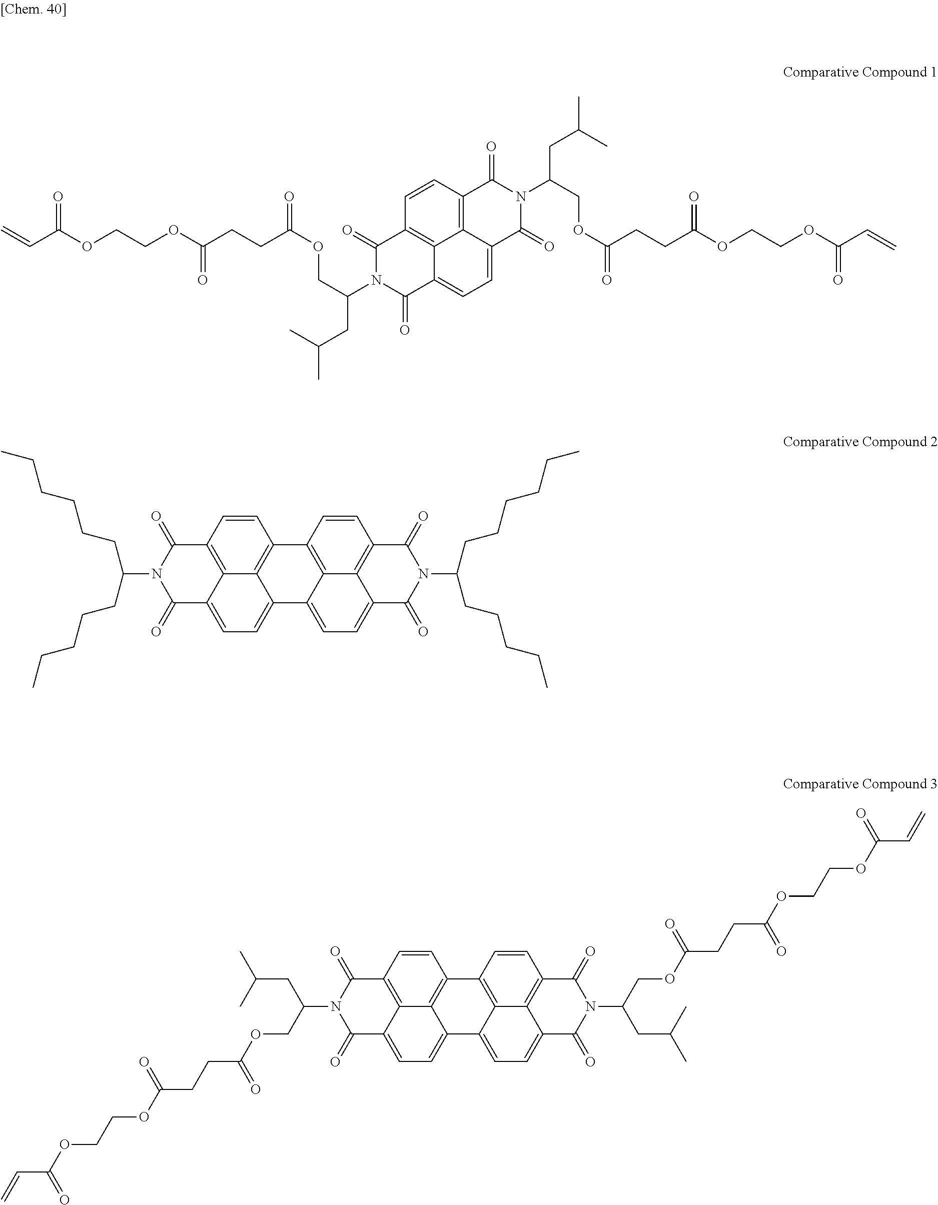

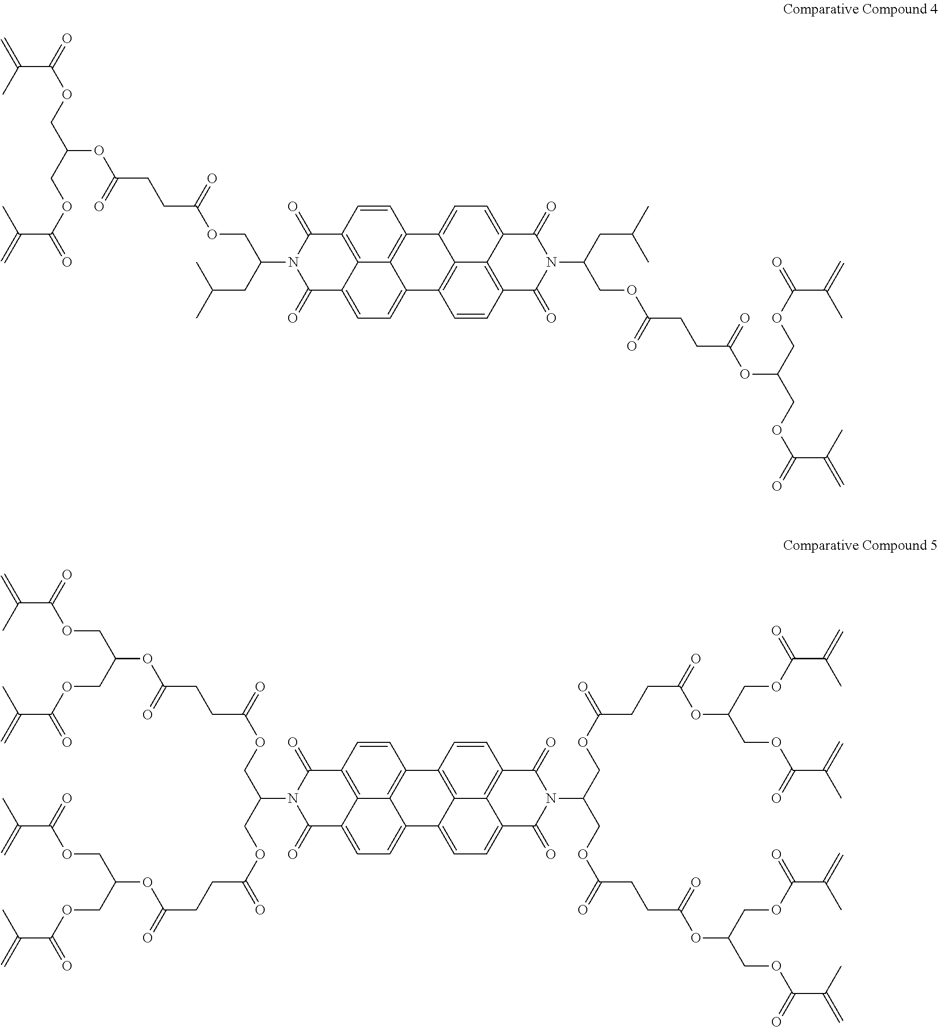

### <Synthesis of Compound 1>

The synthesis scheme of Compound 1 is shown below.

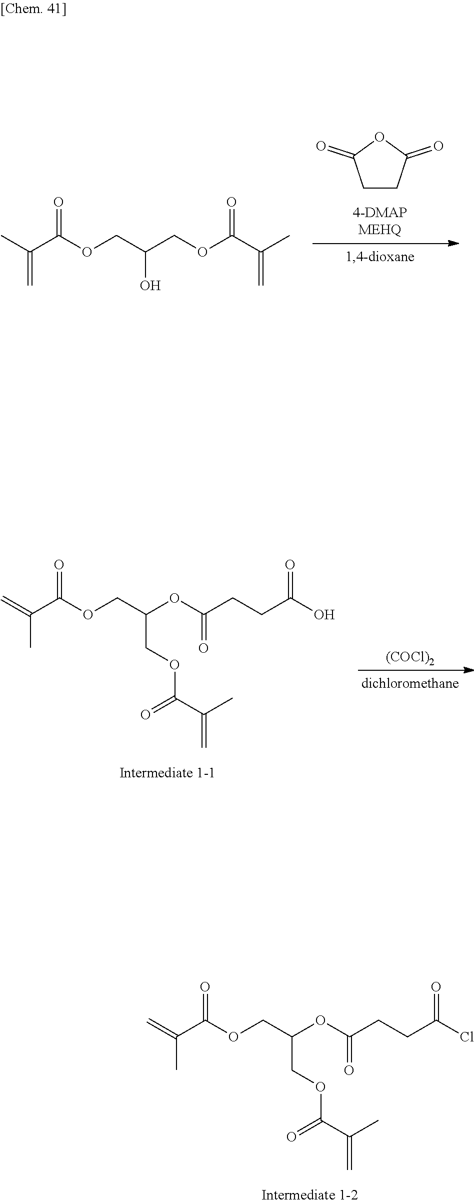

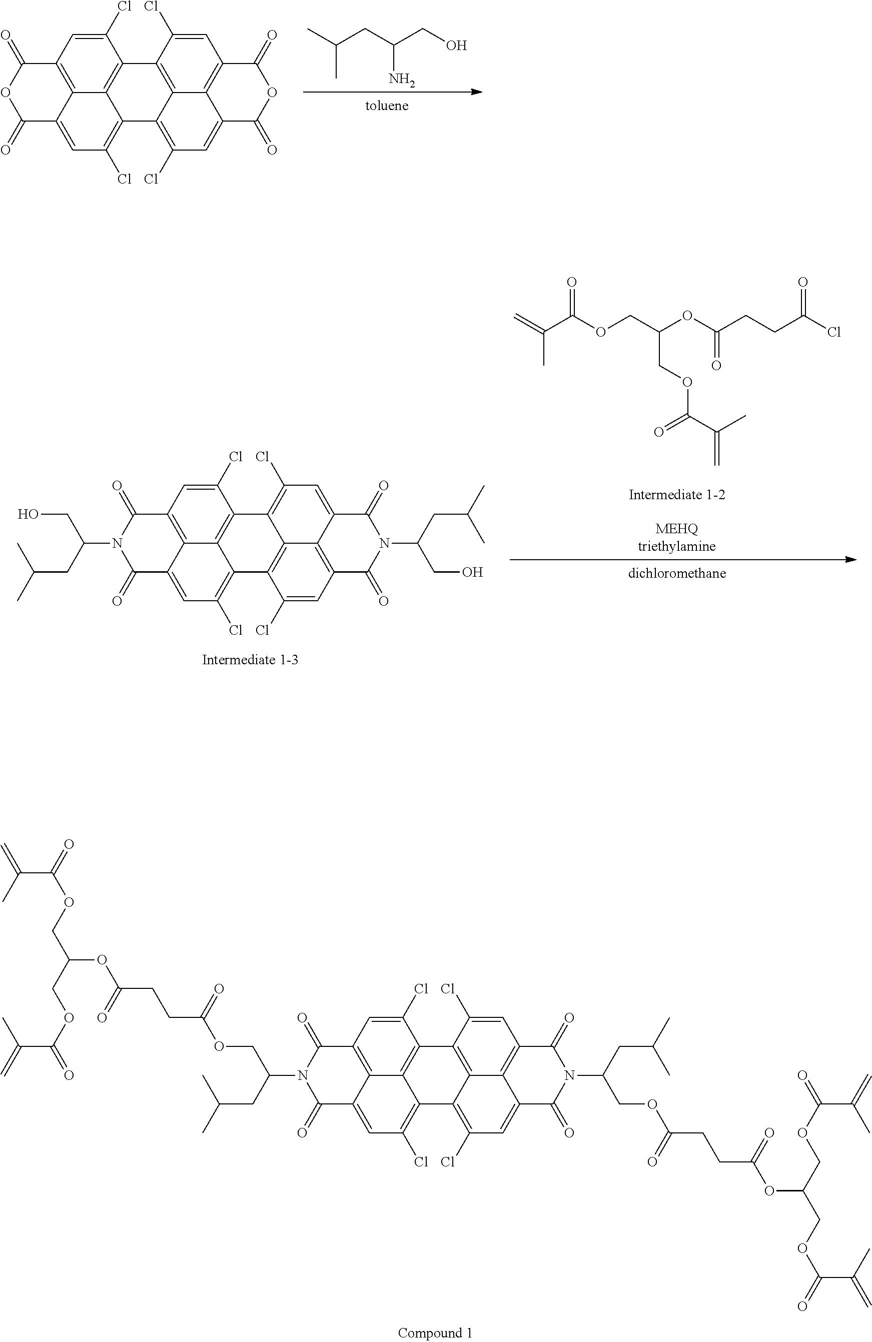

The synthesis procedure for Compound 1 is shown below.

### (Synthesis of Intermediate 1-1)

Under a nitrogen atmosphere, 100 mL of 1,4-dioxane was added to succinic anhydride (11.0 g, 109.5 mmol) and 4-DMAP (4-dimethylaminopyridine, 0.26 g, 2.19 mmol). A solution of glycerol dimethacrylate (25 g, 109.5 mmol) and MEHQ (27 mg, 0.22 mmol) dissolved in 50 mL of 1,4-dioxane was added dropwise to this solution and stirred at 80° C. for 9 hours. After cooling to room temperature, the mixture was poured into 200 mL of water and extracted with dichloromethane. The organic layer was washed with water and then dried with magnesium sulfate. The solid was filtered, the solvent of the filtrate was distilled off under reduced pressure, and the residue was dried to obtain Intermediate 1-1 (yield 30 g, 83%).

### (Synthesis of Intermediate 1-2)

Under a nitrogen atmosphere, 100 mL of dehydrated dichloromethane and 1 mL of dehydrated dimethylformamide were added to Intermediate 1-1 (21.6 g, 65.8 mmol) and cooled on ice. Oxalyl chloride (11.2 mL, 131.6 mmol) was added dropwise thereto, and the mixture was stirred for 2 hours under ice cooling and for 12 hours at room temperature. After distilling off the solvent under reduced pressure, the residue was dried to obtain Intermediate 1-2 (yield 21.5 g, 94%).

### (Synthesis of Intermediate 1-3)

Under a nitrogen atmosphere, 5,6,12,13-Tetrachloroperylo[3,4-cd:9,10-c′d′]dipyran-1,3,8,10-tetrone (13.0 g, 24.5 mmol) and L-(+)-leucinol (7.2 g, 61.3 mmol) were added to 200 mL of toluene and stirred at 110° C. for 9 hours. After cooling to room temperature, the solution was poured into 200 mL of ice water, and 1N hydrochloric acid was added to make the reaction solution acidic. The reaction solution was extracted with toluene and tetrahydrofuran, and the organic layer was washed with water, and then dried with magnesium sulfate. The resulting solid was filtered, and the solvent of the filtrate was distilled off under reduced pressure. The residue was dried to obtain Intermediate 1-3 (yield 11.8 g, 66%).

### (Synthesis of Compound 1)

Under a nitrogen atmosphere, 100 mL of dehydrated dichloromethane and triethylamine (6.6 mL, 47.8 mmol) were added to Intermediate 1-3 (5.8 g, 7.96 mmol) and 4-methoxyphenol (0.05 g), and the mixture was cooled on ice. Intermediate 1-2 (8.3 g, 23.9 mmol) dissolved in 70 mL of dehydrated dichloromethane was added dropwise, and the mixture was stirred for 1 hour under ice cooling and for 12 hours at room temperature. The reaction solution was poured into 200 mL of ice water, extracted with dichloromethane, and the organic layer was washed with water and then dried with magnesium sulfate. The resulting solid was filtered, and the solvent of the filtrate was distilled off under reduced pressure, and the residue was subjected to silica gel column chromatography to obtain Compound 1 (yield 5.7 g, 53%).

### <Synthesis of Compound 2>

The synthesis scheme of Compound 2 is shown below.

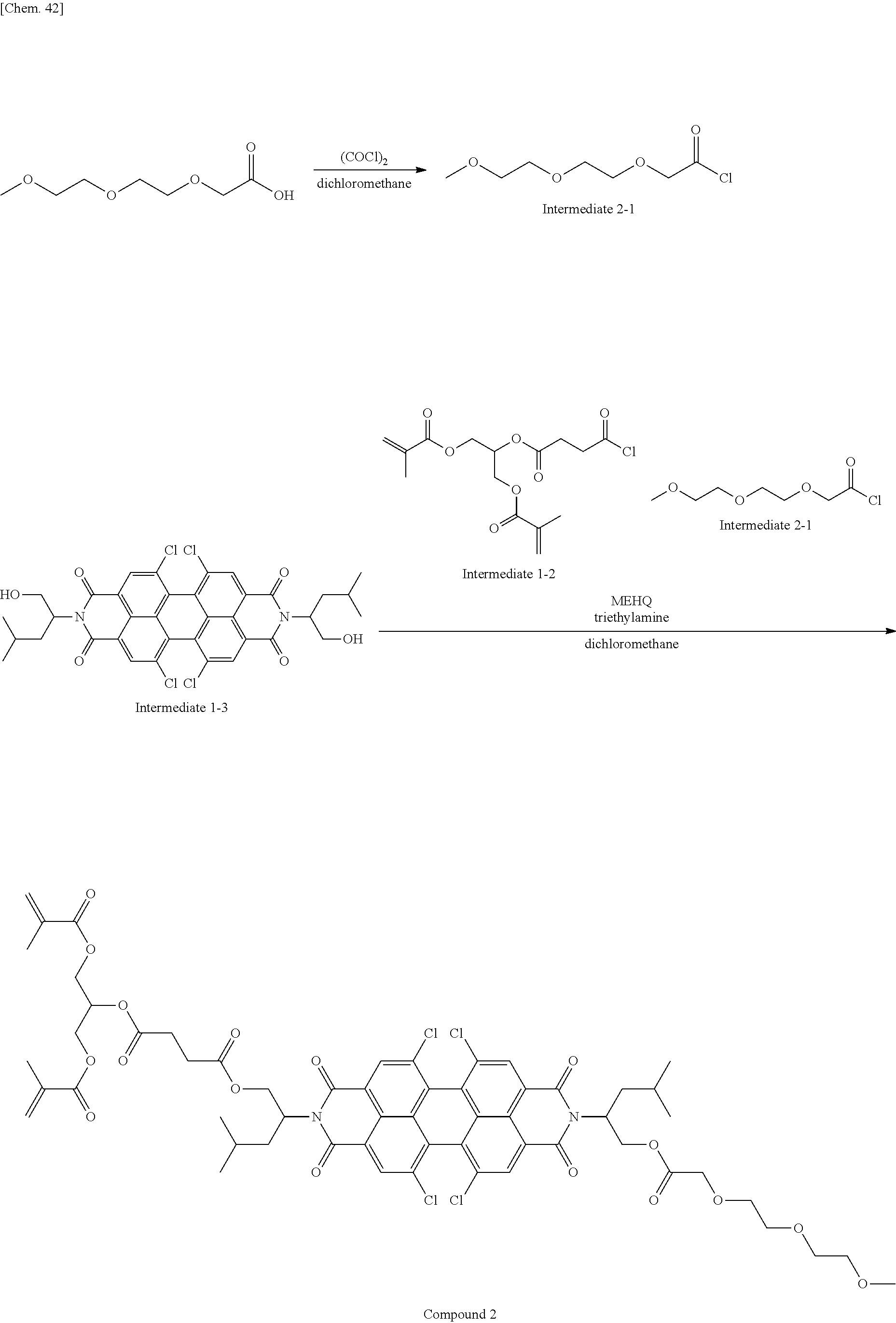

The synthesis procedure for Compound 2 is shown below.

### (Synthesis of Intermediate 2-1)

Under a nitrogen atmosphere, 100 mL of dehydrated dichloromethane was added to [2-(2-methoxyethoxy)ethoxy]acetic acid (6.3 g, 35.3 mmol) and cooled on ice. 0.5 mL of N,N-dimethylformamide was added to this solution, and oxalyl chloride (6.1 mL, 70.7 mmol) was added dropwise, and the mixture was stirred for 1 hour under ice cooling and for 12 hours at room temperature. After distilling off the solvent under reduced pressure, the residue was dried to obtain Intermediate 2-1 (yield 6.9 g, 99%).

### (Synthesis of Compound 2)

Under a nitrogen atmosphere, 150 mL of dehydrated dichloromethane and triethylamine (13.4 mL, 96.6 mmol) were added to Intermediate 1-3 (11.7 g, 16.1 mmol) and 4-methoxyphenol (0.05 g) and cooled on ice. Intermediate 1-2 (8.36 g, 24.1 mmol) and Intermediate 2-1 (4.79 g, 24.1 mmol) dissolved in 50 mL of dehydrated dichloromethane were added dropwise, and the mixture was stirred for 1 hour under ice cooling and for 12 hours at room temperature. The solvent of the reaction solution was distilled off under reduced pressure, and the residue was subjected to silica gel column chromatography to obtain Compound 2 (yield 8.2 g, 42%).

### <Synthesis of Comparative Compound 1>

The synthesis scheme of Comparative Compound 1 is shown below.

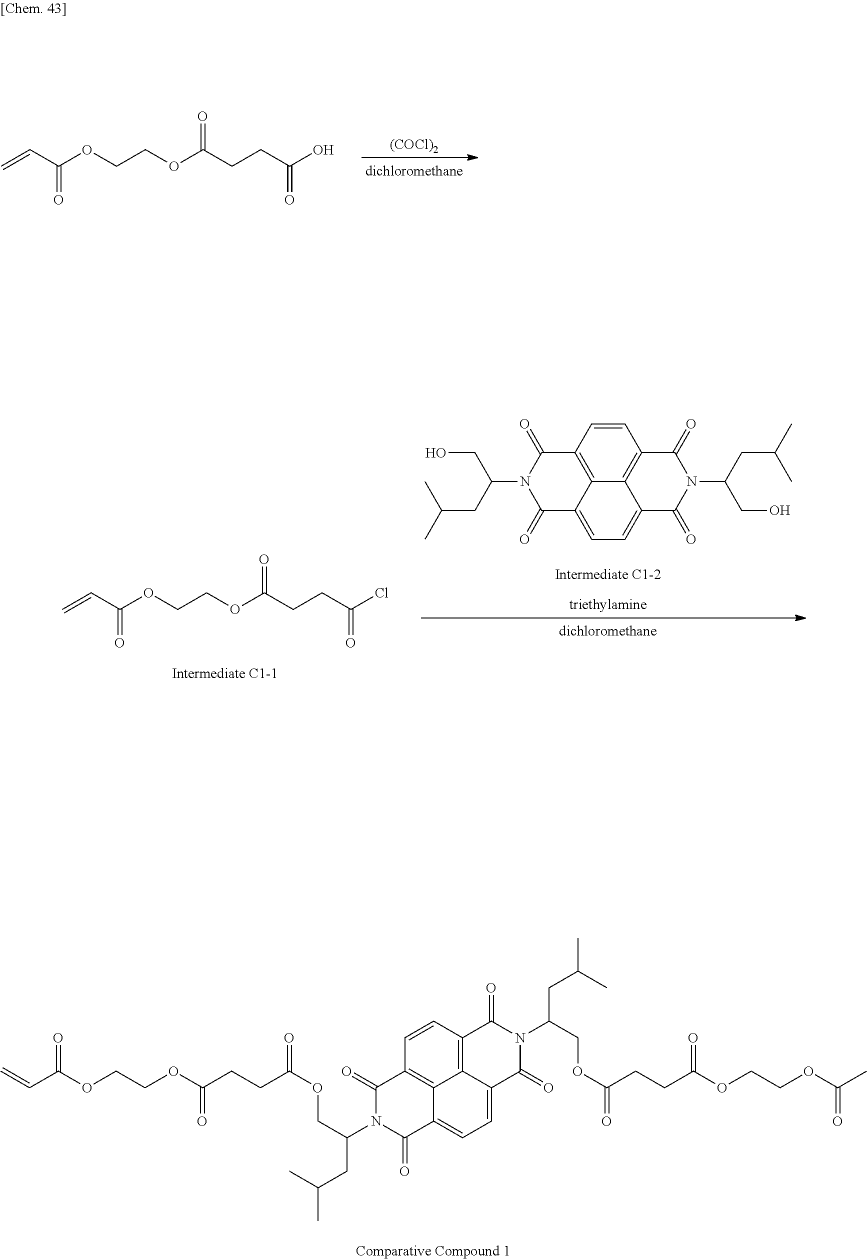 [SMILES: C=CC(=O)OCCOC(=O)CCC(=O)O ; "(COCl)2" ; CC(C)CC(CO)N1C(=O)c2ccc3c4c(ccc(c24)C1=O)C(=O)N(C(CO)CC(C)C)C3=O ; C=CC(=O)OCCOC(=O)CCC(=O)Cl ; C=CC(=O)OCCOC(=O)CCC(=O)OCC(CC(C)C)N1C(=O)c2ccc3c4c(ccc(c24)C1=O)C(=O)N(C(COC(=O)CCC(=O)OCCOC(C)=O)CC(C)C)C3=O]

The synthesis procedure for Comparative Compound 1 is shown below.

### (Synthesis of Intermediate C1-1)

Under a nitrogen atmosphere, 200 mL of dehydrated dichloromethane was added to mono(2-acryloyloxyethyl) succinate (21.7 g, 100.4 mmol) and cooled on ice. Oxalyl chloride (11.2 mL, 130.5 mmol) was added dropwise thereto, and the mixture was stirred for 1 hour under ice cooling and for 12 hours at room temperature. The solvent in the reaction solution was distilled off under reduced pressure, and the residue was dried to obtain Intermediate C1-1 (yield 23 g, 97%).

### (Synthesis of Comparative Compound 1)

Under a nitrogen atmosphere, 50 mL of dehydrated dichloromethane and triethylamine (5.1 mL, 36.8 mmol) were added to Intermediate C1-2 (4.3 g, 9.21 mmol) and cooled on ice. Intermediate C1-1 (4.8 g, 20.3 mmol) dissolved in 50 mL of dehydrated dichloromethane was added dropwise thereto, and stirred for 1 hour under ice cooling and at room temperature for 1 hour. The reaction solution was poured into 100 mL of water, extracted with dichloromethane, and the organic layer was washed with water and then dried with magnesium sulfate. The obtained solid was filtered, the solvent of the filtrate was distilled off under reduced pressure, and the residue was subjected to silica gel column chromatography to obtain Comparative Compound 1 (yield 3.5 g, 45%).

### <Synthesis of Comparative Compound 2>

The synthesis scheme of Comparative Compound 2 is shown below.

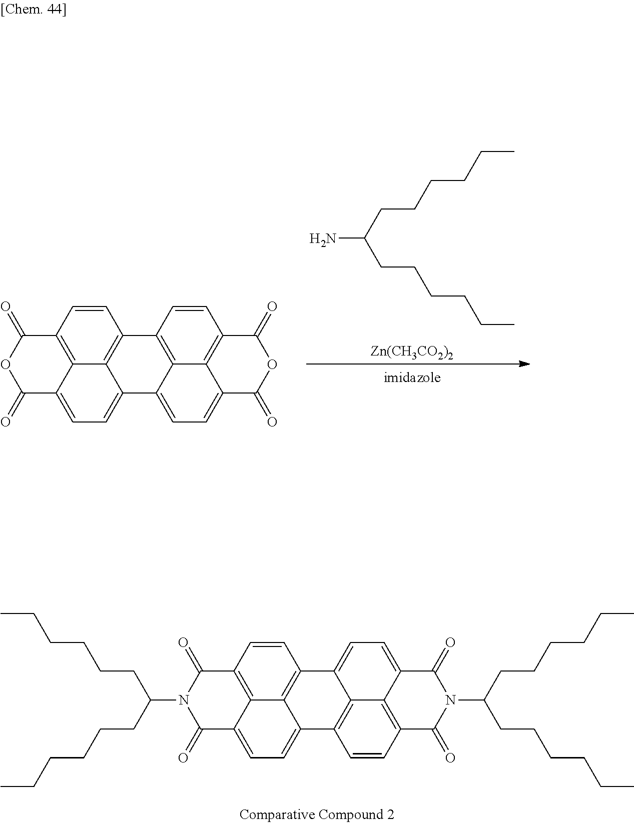 [SMILES: O=C1OC(=O)c2ccc3c4ccc5c6c(ccc(c7ccc1c2c73)c64)C(=O)OC5=O ; CCCCCCC(N)CCCCCC ; "Zn(CH3CO2)2" ; CCCCCCC(CCCCCC)N1C(=O)c2ccc3c4ccc5c6c(ccc(c7ccc(c2c37)C1=O)c64)C(=O)N(C(CCCCCC)CCCCCC)C5=O]

The synthesis procedure for Comparative Compound 2 is shown below.

### (Synthesis of Comparative Compound 2)

Under a nitrogen atmosphere, 3,4,9,10-perylenetetracarboxylic dianhydride (2.62 g, 6.69 mmol), zinc acetate (0.92 g, 5.02 mmol), 10 g of imidazole, and tridecane-7-amine (4.00 g, 20.1 mmol) were stirred at 160° C. for 5 hours. After cooling to room temperature, the mixture was dissolved in dichloromethane, and the solution was poured into 200 mL of ice water, and 1N hydrochloric acid was added to make the reaction solution acidic. Extraction was performed with dichloromethane, and the organic layer was washed with water and then dried with magnesium sulfate. The obtained solid was filtered, the solvent of the filtrate was distilled off under reduced pressure, and the residue was subjected to silica gel column chromatography to obtain Comparative Compound 2 (yield 4.2 g, 83%).

### <Synthesis of Comparative Compound 3>

The synthesis scheme of Comparative Compound 3 is shown below.

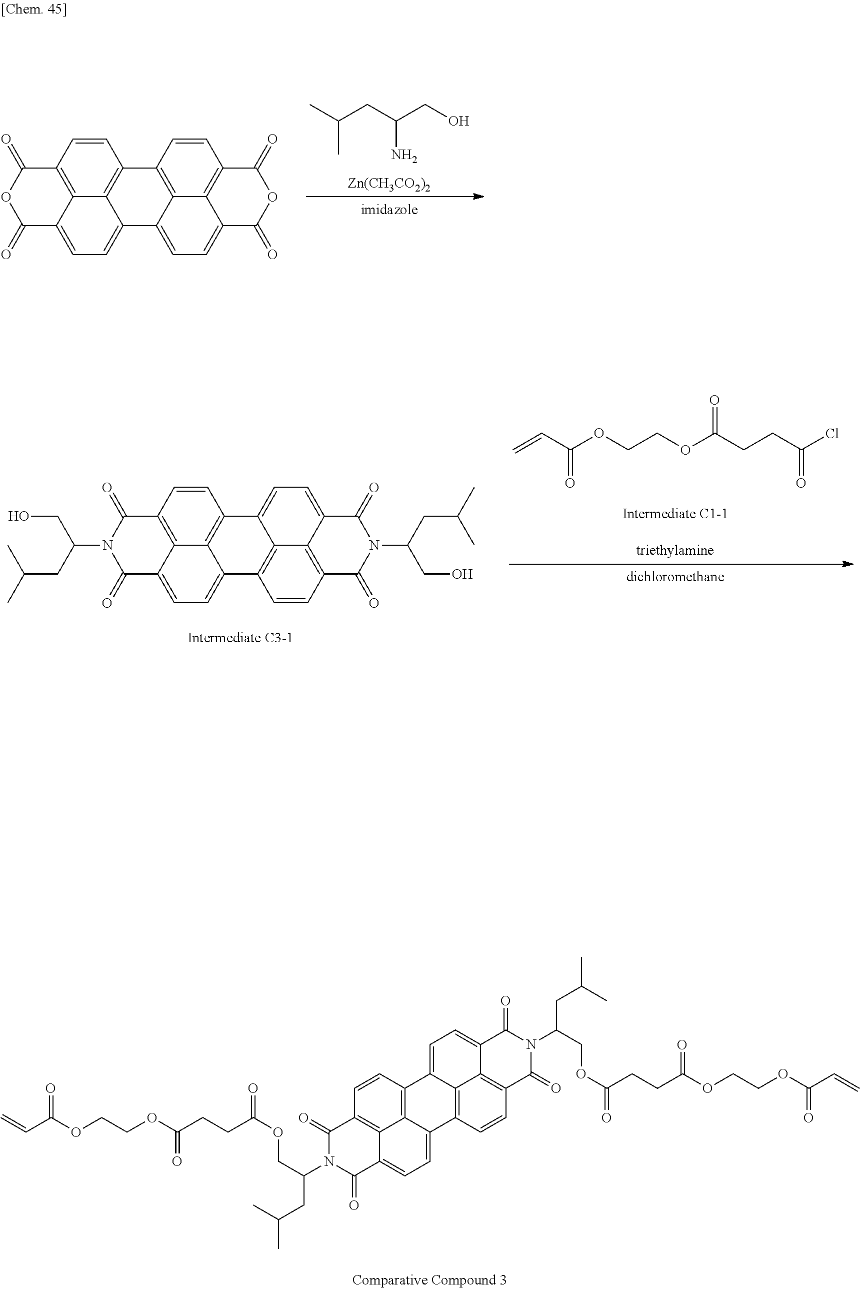

The synthesis procedure for Comparative Compound 3 is shown below.

### (Synthesis of Intermediate C3-1)

Under a nitrogen atmosphere, 3,4,9,10-perylenetetracarboxylic dianhydride (11.4 g, 29.0 mmol), zinc acetate (5.32 g, 29.0 mmol), 50 g of imidazole, and L-(+)-leucinol (8.5 g, 72.5 mmol) were stirred at 160° C. for 7 hours. After cooling to room temperature, the mixture was dissolved in dichloromethane and the solution was poured into 200 mL of ice water, and 1N hydrochloric acid was added to make the reaction solution acidic. The mixture was extracted with dichloromethane, and the organic layer was washed with water and then dried with magnesium sulfate. The obtained solid was filtered, the solvent of the filtrate was distilled off under reduced pressure, and the residue was subjected to silica gel column chromatography to obtain Intermediate C3-1 (yield 12.0 g, 70%).

### (Synthesis of Comparative Compound 3)

Under a nitrogen atmosphere, 100 mL of dehydrated dichloromethane and triethylamine (1.5 mL, 10.8 mmol) were added to Intermediate C3-1 (1.6 g, 2.71 mmol) and cooled on ice. Intermediate C1-1 (1.4 g, 5.96 mmol) dissolved in 10 mL of dehydrated dichloromethane was added dropwise, and the mixture was stirred for 1 hour under ice cooling and at room temperature for 1 hour. The reaction solution was poured into 100 mL of water, extracted with dichloromethane, and the organic layer was washed with water and then dried with magnesium sulfate. The obtained solid was filtered, the solvent of the filtrate was distilled off under reduced pressure, and the residue was subjected to silica gel column chromatography to obtain Comparative Compound 3 (yield 2.3 g, 85%).

### <Synthesis of Comparative Compound 4>

The synthesis scheme of Comparative Compound 4 is shown below.

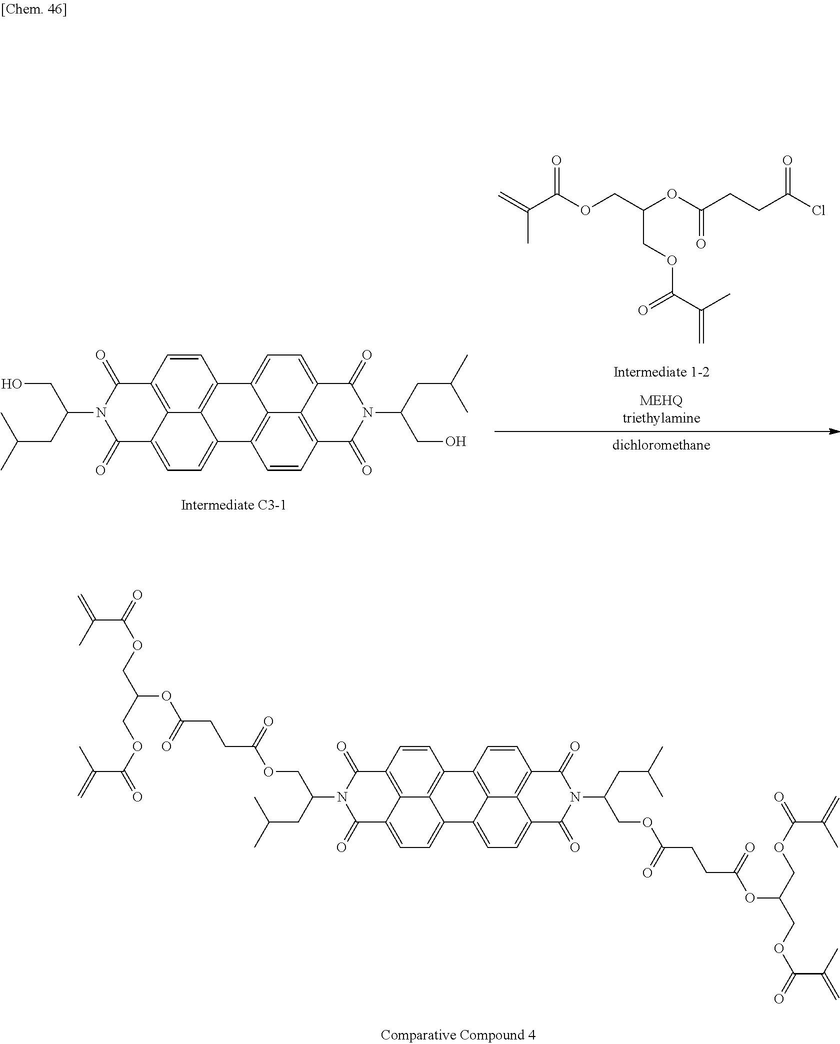 [SMILES: C=C(C)C(=O)OCC(COC(=O)C(=C)C)OC(=O)CCC(=O)Cl ; CC(C)CC(CO)N1C(=O)c2ccc3c4ccc5c6c(ccc(c7ccc(c2c37)C1=O)c64)C(=O)N(C(CO)CC(C)C)C5=O ; C=C(C)C(=O)OCC(COC(=O)C(=C)C)OC(=O)CCC(=O)OCC(CC(C)C)N1C(=O)c2ccc3c4ccc5c6c(ccc(c7ccc(c2c37)C1=O)c64)C(=O)N(C(COC(=O)CCC(=O)OC(COC(=O)C(=C)C)COC(=O)C(=C)C)CC(C)C)C5=O]

The synthesis procedure of Comparative Compound 4 is shown below.

### (Synthesis of Comparative Compound 4)

Under a nitrogen atmosphere, 150 mL of dehydrated dichloromethane and triethylamine (29.5 mL, 213 mmol) were added to Intermediate C3-1 (21.0 g, 35.5 mmol) and 4-methoxyphenol (0.05 g), and the mixture was cooled on ice. Intermediate 1-2 (36.9 g, 107 mmol) dissolved in 50 mL of dehydrated dichloromethane was added dropwise, and the mixture was stirred for 1 hour under ice cooling and for 2 hours at room temperature. The reaction solution was poured into 100 mL of water, extracted with dichloromethane, and the organic layer was washed with water and then dried with magnesium sulfate. The obtained solid was filtered, the solvent of the filtrate was distilled off under reduced pressure, and the residue was subjected to silica gel column chromatography to obtain Comparative Compound 4 (yield 32.5 g, 76%).

### <Synthesis of Comparative Compound 5>

The synthesis scheme of Comparative Compound 5 is shown below.

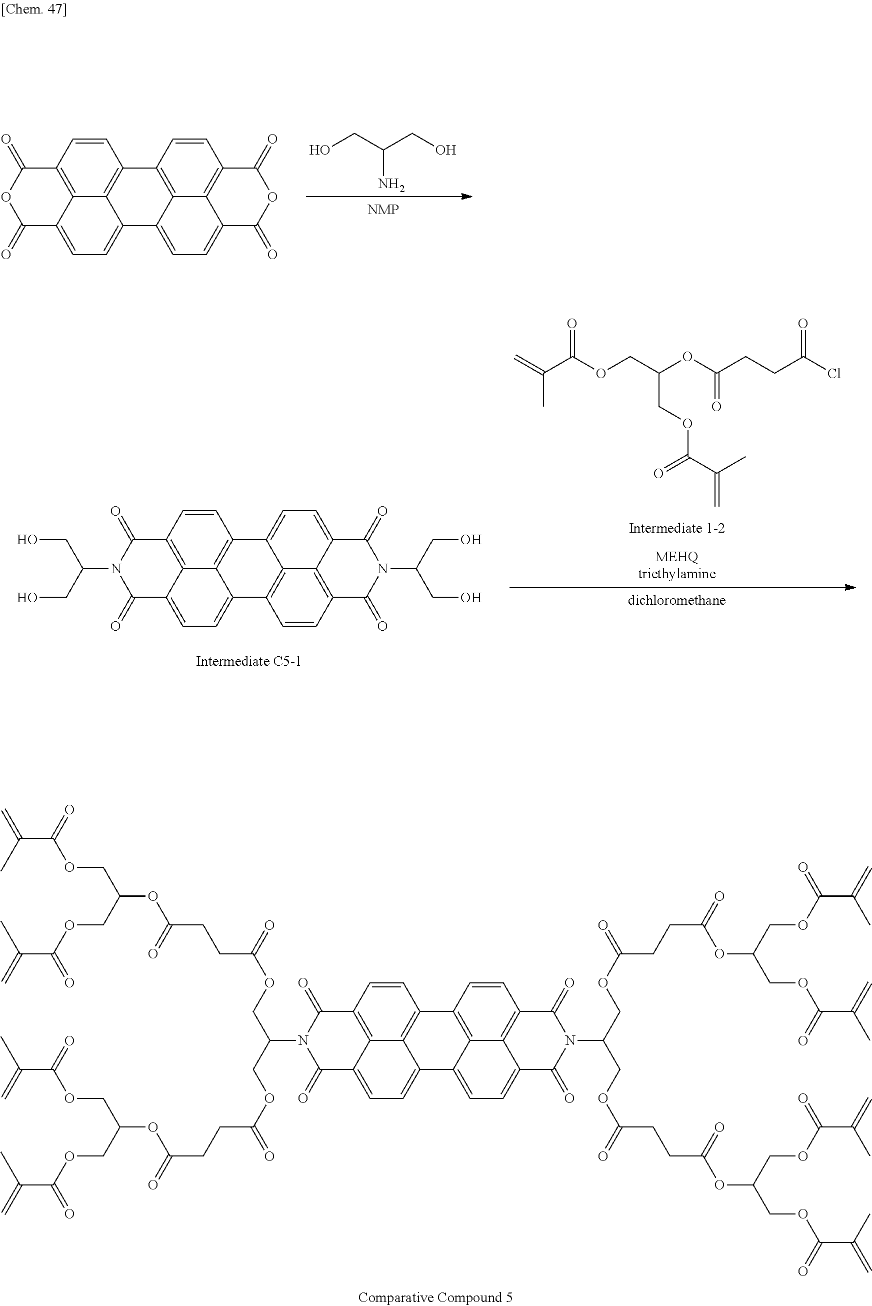 [SMILES: O=C1OC(=O)c2ccc3c4ccc5c6c(ccc(c7ccc1c2c73)c64)C(=O)OC5=O ; NC(CO)CO ; C=C(C)C(=O)OCC(COC(=O)C(=C)C)OC(=O)CCC(=O)Cl ; O=C1c2ccc3c4ccc5c6c(ccc(c7ccc(c2c37)C(=O)N1C(CO)CO)c64)C(=O)N(C(CO)CO)C5=O ; C=C(C)C(=O)OCC(COC(=O)C(=C)C)OC(=O)CCC(=O)OCC(COC(=O)CCC(=O)OC(COC(=O)C(=C)C)COC(=O)C(=C)C)N1C(=O)c2ccc3c4ccc5c6c(ccc(c7ccc(c2c37)C1=O)c64)C(=O)N(C(COC(=O)CCC(=O)OC(COC(=O)C(=C)C)COC(=O)C(=C)C)COC(=O)CCC(=O)OC(COC(=O)C(=C)C)COC(=O)C(=C)C)C5=O]

The synthesis procedure of Comparative Compound 5 is shown below.

### (Synthesis of Intermediate C5-1)

Under a nitrogen atmosphere, 3,4,9,10-perylenetetracarboxylic dianhydride (7.6 g, 19.4 mmol), 2-amino-1,3-propanediol (3.5 g, 38.7 mmol), and 200 mL of NMP were stirred at 150° C. for 8 hours. After cooling to room temperature, the solution was poured into 400 mL of ice water, and 1N hydrochloric acid was added to make the reaction solution acidic. The obtained solid was filtered, and the filtered product was washed with water. The filtered product was dried to obtain Intermediate C5-1 (yield 10.0 g, 96%).

### (Synthesis of Comparative Compound 5)

Under a nitrogen atmosphere, 200 mL of dehydrated dichloromethane and triethylamine (30.9 mL, 223 mmol) were added to Intermediate C5-1 (10.0 g, 18.6 mmol) and 4-methoxyphenol (0.05 g), and the mixture was cooled on ice. Intermediate 1-2 (38.6 g, 111 mmol) dissolved in 100 mL of dehydrated dichloromethane was added dropwise, and the mixture was stirred for 1 hour under ice cooling and for 12 hours at room temperature. The reaction solution was poured into 200 mL of water, extracted with dichloromethane, and the organic layer was washed with water and then dried with magnesium sulfate. The obtained solid was filtered, the solvent of the filtrate was distilled off under reduced pressure, and the residue was subjected to silica gel column chromatography to obtain Comparative Compound 5 (Yield 6.80 g, 21%).

### [Evaluation of Solubility in Organic Solvent]

Compounds 1 to 2 or Comparative Compounds 1 to 5 were respectively added to a mixed solvent of toluene/2-propanol=3/7 (mass ratio) at room temperature (25° C.) so as to have a concentration of 8% by mass, and stirred for 10 minutes with a rotor without heating or cooling. The state of dissolution was then visually observed, and the solubility was evaluated according to the following criteria.

When any residue was observed, the mixture was heated in a water bath at a constant temperature (40° C.) for 10 minutes as described below, and the state of dissolution was visually observed again to evaluate the solubility. “S” or “A” was judged to have particularly excellent solubility in organic solvents. The evaluation results are shown in Table 1.
S: The compound dissolved completely at room temperature.
A: A small amount of residue was observed at room temperature, but the residue dissolved completely when heated at 40° C. for 10 minutes or less.
B: Some residue was observed at room temperature, but the residue dissolved completely when heated at 40° C. for 10 minutes or more.
C: When heated at 40° C. for 10 minutes or more, some residue was observed.

Electrophotographic photoreceptors were produced by the following method using the above compounds having other than “C” in the evaluation of solubility in the above organic solvent, and the residual potential and potential retention rate were evaluated by the following method.

The evaluation results are shown in Table 1.

### [Production of Electrophotographic Photoreceptor]

### <Preparation of Coating Liquid P1 for Forming Undercoat Layer>

20 parts of D-type titanyl phthalocyanine, which exhibits a clear peak at a diffraction angle of 2θ=27.3°±0.2° in powder X-ray diffraction using CuKα rays, and 280 parts of 1,2-dimethoxyethane were mixed, and the mixture was ground with a sand grind mill for 2 hours to conduct atomization dispersion treatment. Further, 400 parts of a 2.5% 1,2-dimethoxyethane solution of polyvinyl butyral (manufactured by Denki Kagaku Kogyo Co., Ltd., product name “Denka Butyral” #6000C) and 170 parts of 1,2-dimethoxyethane were added thereto, and mixed to prepare a coating liquid P1 for forming an undercoat layer having a solid concentration of 3.4% by mass.

### <Preparation of Coating Liquid Q1 for Forming Single Layer Type Photosensitive Layer>

2.6 parts of D-type titanyl phthalocyanine, which exhibits a clear peak at a diffraction angle of 2θ=27.3°±0.2° in powder X-ray diffraction using CuKα rays, 1.3 parts of perylene pigment 1 having the following structure, 0.5 parts of polyvinyl butyral resin, 90 parts of the following hole transporting material (HTM48, molecular weight 748), 70 parts of the following electron transporting material (ET-2, molecular weight 424.2), 100 parts of polycarbonate resin having a biphenyl structure, and 0.05 parts of silicone oil (manufactured by Shin-Etsu Silicone Co., Ltd., product name KF-96) as a leveling agent were mixed with 793.35 parts of a mixed solvent (90% by mass of THF and 10% by mass of TL) of tetrahydrofuran (hereinafter, appropriately abbreviated as THF) and toluene (hereinafter, appropriately abbreviated as TL) to prepare a coating liquid Q1 for forming a single layer type photosensitive layer having a solid content concentration of 25% by mass.

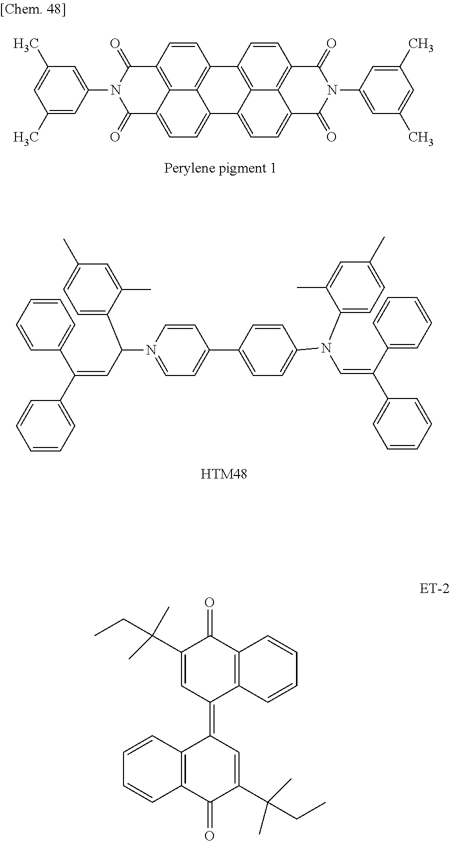

### <Preparation of Coating Liquids S1 to S5 for Forming Protective Layer>

A curable compound (dipentaerythritol polyacrylate: manufactured by Shin-Nakamura Chemical Co., Ltd., product name “NK Ester A-DPH”) or a polycarbonate resin (viscosity average molecular weight of 40,000) having a repeating unit of the following structure dissolved in a mixed solvent of toluene/2-propanol was mixed with benzophenone and Omnirad TPO H (2,4,6-trimethylbenzoyl-diphenylphosphine oxide) as polymerization initiators, and Compounds 1 to 2 or Comparative Compound 1, 4 or 5 as the electron transporting compound respectively to obtain coating liquids S1 to S5 (having a solid concentration of approximately 8.0% by mass) for forming a protective layer having the following compositions.

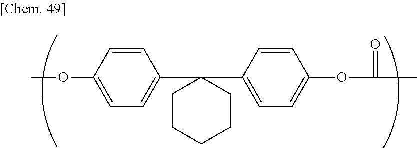

S1, S2, S4, S5: Electron transporting compound/A-DPH/benzophenone/Omnirad TPO H=100/50/1/2 (mass ratio), solvent composition toluene/2-propanol=3/7 (mass ratio)
S3: Electron transporting compound/polycarbonate resin/benzophenone/Omnirad TPO H=100/100/2/1 (mass ratio), solvent composition toluene/2-propanol=3/7 (mass ratio)

Comparative Compounds 2 and 3 both had poor solubility, so they were not possible to prepare coating liquids for forming a protective layer.

### <Production of Single Layer Photoreceptors A1 to A5>

Single layer photoreceptors were produced by the following procedure.

An aluminum cylinder of 30 mmφ and a length of 244 mm having a surface subjected to cutting treatment was coated with the coating liquid P1 for forming an undercoat layer by dip coating, and thus an undercoat layer was provided in such a manner that the thickness after drying became 0.3 μm. The coating liquid Q1 for forming a single layer type photosensitive layer was applied on the undercoat layer by dip coating and dried at 100° C. for 24 minutes, and thus a single layer photosensitive layer was provided in such a manner that the thickness after drying became 32 μm. The coating liquids S1 to S5 for forming a protective layer were respectively applied on the single layer photosensitive layer by ring coating and soon after application, LED light of 365 nm was applied at an intensity of 0.9 W/cm² for 30 seconds for photoreceptors A1 and A4, at an intensity of 0.9 W/cm² for 60 seconds for photoreceptors A2 and A5, and at an intensity of 0.9 W/cm² for 120 seconds for photoreceptor A3, respectively, while the photoreceptor was rotated at 60 rpm in a nitrogen atmosphere, to provide a protective layer in such a manner that the film thickness after curing became 1.5 μm. Thus, photoreceptors A1 to A5 were produced, respectively.

### [Measurement of Residual Potential]

The obtained photoreceptors A1 to A5 were attached to an electrophotographic property evaluation device manufactured in accordance with the measurement standards of the Society of Electrophotography of Japan (described in Electrophotographic Technology Basics and Applications Continued, edited by the Society of Electrophotography of Japan, Corona Publishing Co., Ltd., pp. 404 to 405), and the electrical properties in the cycle of charging, exposure, potential measurement, and static elimination were measured as follows.

First, the grid voltage was adjusted to charge the photoreceptor so that the initial surface potential (V0) was +700V. Next, the sample was irradiated an exposure light of 1.3 μJ/cm², and the residual potential was measured (VL) was measured 60 milliseconds after irradiation. The exposure light used was a monochromatic light of 780 nm emitted from a halogen lamp and converted by an interference filter. The measurement was performed in an environment with a temperature of 25° C. and a relative humidity of 50% (N/N environment).

The smaller the absolute value of the residual potential (V), the better the result, since it means that the charge has been sufficiently transported to reduce the potential.

### [Measurement of Potential Retention Rate]

The potential retention rate of the each obtained photoreceptors A1 to A5 was measured using the following method.

The sample was attached to an electrophotographic property evaluation device manufactured in accordance with the measurement standards of the Society of Electrophotography of Japan (described in Electrophotographic Technology Basics and Applications Continued, edited by the Society of Electrophotography of Japan, Corona Publishing Co., Ltd., pp. 404 to 405), and the electrical properties in the cycle of charging, exposure, potential measurement, and static elimination were measured as follows.

To evaluate the electrical properties, the dark decay (DDR) (%) was measured after charging to +700V and leaving for 5 seconds. The measurement was performed in an environment with a temperature of 25° C. and a relative humidity of 50% (N/N environment).

The potential retention rate is shown in Table 1. The potential retention rate represents the surface potential retention rate (%) when a photoreceptor having a charged surface is left for a certain period of time. A higher surface potential retention rate (%) indicates better results, as the potential is maintained even over time and charging properties are good.

In this Example, a residual potential of 200V or less was considered to be “passed”, and a potential retention rate of 60% or more was considered to be “passed”.

TABLE 1

| | Electron Transporting | | Photoreceptor | | |
|---|---|---|---|---|---|
| | Compound | | | Residual | Potential |
| | | Solu- | | Ptential | Rtention |
| | Type | bility | Type | (V) | Rate (%) |
| Example 1 | Compound 1 | S | Photore- | 110 | 85.6 |
| | | | ceptor A1 | | |
| Example 2 | Compound 2 | S | Photore- | 68 | 83.4 |
| | | | ceptor A2 | | |
| Comparative | Comparative | B | Photore- | 261 | 84.9 |
| Example 1 | Compound 1 | | ceptor A3 | | |
| Comparative | Comparative | C | — | Cannot be Ealuated | |
| Example 2 | Compound 2 | | | | |
| Comparative | Comparative | C | — | Cannot be Ealuated | |
| Example 3 | Compound 3 | | | | |
| Comparative | Comparative | S | Photore- | 236 | 32.2 |
| Example 4 | Compound 4 | | ceptor A4 | | |
| Comparative | Comparative | A | Photore- | 316 | 84.9 |
| Example 5 | Compound 5 | | ceptor A5 | | |

From Table 1, it can be seen that the compounds according to the first embodiment and second embodiment of the present invention have excellent solubility in organic solvents, and can form an electron transporting protective layer with good film forming property that can impart excellent electrical properties such as residual potential property, potential retention rate, and the like to the photoreceptor produced.

On the other hand, Comparative Compound 1, in which the electron transporting skeleton is not a perylene diimide skeleton and contains no halogen atoms, does not have sufficient organic solvent solubility and also has significantly poor residual potential property.

It can be seen that when the electron transporting skeleton is a perylene diimide skeleton but contains no halogen atoms, the organic solvent solubility differs depending on the presence or absence of a linking group (3A) and the number of polymerizable functional groups.

Among them, Comparative Compounds 4 and 5 have excellent solubility in organic solvents but poor residual potential property, and Comparative Compound 4 also has poor potential retention rate.

Although the present invention has been described in detail using specific embodiments, it is clear to those skilled in the art that various changes can be made within the scope of achieving the effects of the invention.

The present application is based on Japanese Patent Application No. 2022-212000 and Japanese Patent Application No. 2022-212003 filed on Dec. 28, 2022, the entire contents of which are incorporated herein by reference.

### REFERENCE SIGNS LIST

1: Present electrophotographic photoreceptor
2: Charging device
3: Exposure device
4: Developing device
5: Transfer device
6: Cleaning device
7: Fixing device

1. A compound having two or more polymerizable functional groups in one molecule and represented by the following formula (1),
wherein the polymerizable functional groups are selected from the following formulae (M1) to (M7): 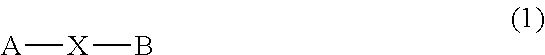
in formula (1), X represents a perylene diimide skeleton represented by the following formula (2),
A and B represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents, an aromatic heterocyclic group which may have one or more substituents, or a group represented by the following formula (3), A and B may be the same or different from each other, 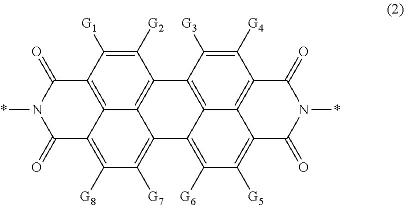
in formula (2), G₁ to G₈ each independently represent a hydrogen atom, a deuterium atom, a halogen atom, an alkyl group which may have one or more substituents, or an alkoxy group which may have one or more substituents. However, at least one of G₁ to G₈ is a halogen atom, * represents a bond to A or B, 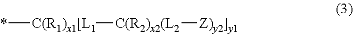
in formula (3), * represents a bond to X,
R₁ and R₂ each independently represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents, or an aromatic heterocyclic group which may have one or more substituents,
L₁ and L₂ each independently represent a direct bond or a divalent group,
Z represents a hydrogen atom, an alkyl group, an alkoxy group, an amide group, or a polymerizable functional group,
x1+y1=3, where x1 is an integer from 0 to 2 and y1 is an integer from 1 to 3; x2+y2=3, where x2 is an integer from 0 to 2 and y2 is an integer from 1 to 3; wherein when x1 is an integer of 2 or more, R₁ may be the same or different from each other; when y1 is an integer of 2 or more, each of R₂, x2, y2, L₁, L₂ and Z may be the same or different from each other; when x2 is an integer of 2 or more, R₂ may be the same or different from each other; and, when y2 is an integer of 2 or more, each of L₂ and Z may be the same or different from each other, 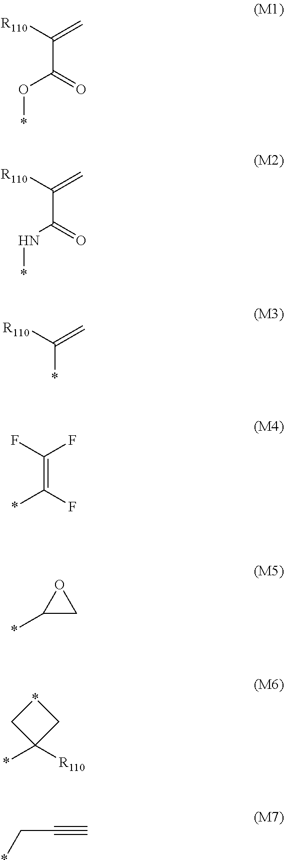
in formulae (M1) to (M7), R₁₁₀ represents a hydrogen atom or an alkyl group which may have one or more substituents, and * represents a bonding position,

2. The compound according to claim 1, wherein L₁ and L₂ in formula (3) are each independently an alkylene group, a divalent group having a ketone group, a divalent group having an ether bond, a divalent group having an ester bond, or a group wherein these are linked.

3. A compound having at least one structure represented by the following formula (3A) in one molecule, and represented by the following formula (1), 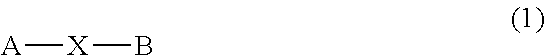
in formula (1), X represents a perylene diimide skeleton represented by the following formula (2),
A and B represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents, an aromatic heterocyclic group which may have one or more substituents, a group represented by the following formula (3), or a group represented by the following formula (3B). A and B may be the same or different from each other, 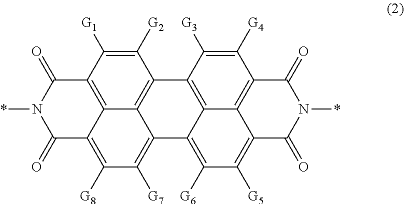
in formula (2), G₁ to G₈ each independently represent a hydrogen atom, a deuterium atom, a halogen atom, an alkyl group which may have one or more substituents, an alkoxy group which may have one or more substituents, or a group represented by the following formula (3B). However, at least one of G₁ to G₈ is a halogen atom. * represents a bond to A or B, 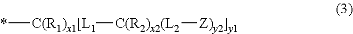
in formula (3), * represents a bond to X.
R₁ and R₂ each independently represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents, an aromatic heterocyclic group which may have one or more substituents, or a group represented by the following formula (3B),
L₁ and L₂ each independently represent a direct bond or a divalent group,
Z represents a hydrogen atom, an alkyl group, an alkoxy group, an amide group, or a polymerizable functional group,
x1+y1=3, where x1 is an integer from 0 to 2 and y1 is an integer from 1 to 3; x2+y2=3, where x2 is an integer from 0 to 2 and y2 is an integer from 1 to 3; wherein when x1 is an integer of 2 or more, R₁ may be the same or different from each other; when y1 is an integer of 2 or more, each of R₂, x2, y2, L₁, L₂ and Z may be the same or different from each other; when x2 is an integer of 2 or more, R₂ may be the same or different from each other; and when y2 is an integer of 2 or more, each of L₂ and Z may be the same or different from each other, 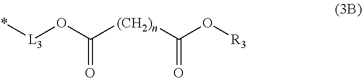
in formula (3B), * represents a bond to any atom in the formulae (1) to (3), and n is an integer of 1 or more,
L₃ represents a direct bond or a divalent group,
R₃ represents a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents, an aromatic heterocyclic group which may have one or more substituents, or a polymerizable functional group, 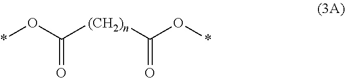
in formula (3A), * represents a bond to any atom in the formulae (1) to (3), and n is an integer of 1 or more.

4. The compound according to claim 3, wherein at least one of L₁ and L₂ in the formula (3) is a divalent group, and the divalent group is a group represented by the formula (3A).

5. The compound according to claim 3, wherein the compound has at least one polymerizable functional group in one molecule.

6. The compound according to claim 5, wherein the polymerizable functional group is selected from the following formulae (M1) to (M7), 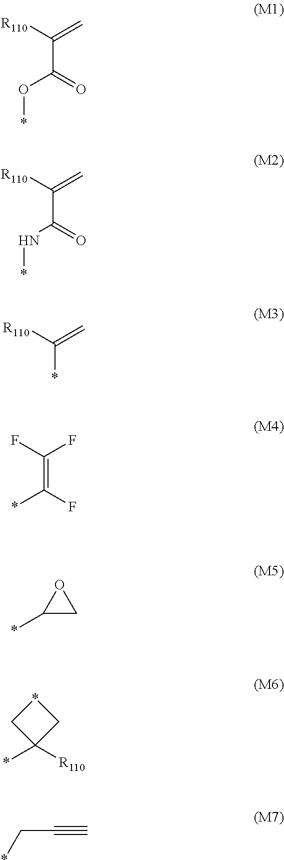
in formulae (M1) to (M7), R₁₁₀ represents a hydrogen atom or an alkyl group which may have one or more substituents, and * represents a bonding position.

7. The compound according to claim 1, wherein at least one of A and B in the formula (1) is a group represented by the above formula (3).

8. The compound according to claim 1, wherein two or more of G₁ to G₈ are halogen atoms.

9. The compound according to claim 8, wherein four or more of G₁ to G₈ are halogen atoms.

10. The compound according to claim 1, wherein R₁ in the formula (3) is an alkyl group which may have one or more substituents.

11. A composition comprising the compound according to claim 1 and a polymerizable compound having no electron transporting skeleton.

12. The composition according to claim 11, further comprising an electron donating compound.

13. An electrophotographic photoreceptor comprising:
a conductive support;
a photosensitive layer and a protective layer formed in this order on the conductive support,
wherein the protective layer comprises a polymer of the compound according to claim 1.

14. An electrophotographic photoreceptor comprising:
a conductive support;
a photosensitive layer and a protective layer formed in this order on the conductive support,
wherein the protective layer comprises the compound according to claim 3.

15. The compound according to claim 3, wherein at least one of A and B in the formula (1) is a group represented by the above formula (3).

16. The compound according to claim 3, wherein two or more of G₁ to G₈ are halogen atoms.

17. The compound according to claim 16, wherein four or more of G₁ to G₈ are halogen atoms.

18. The compound according to claim 3, wherein R₁ in the formula (3) is an alkyl group which may have one or more substituents.

19. A composition comprising the compound according to claim 3 and a polymerizable compound having no electron transporting skeleton.

20. The composition according to claim 19, further comprising an electron donating compound.
